(19) United States
(12) Patent Application Publication
(10) Pub. No.: US 2026/0366045 A1
(43) Pub. Date: Nov. 19, 2026

(54) GENETICALLY MODIFIED YEAST AND FERMENTATION PROCESSES FOR THE PRODUCTION OF XYLITOL

(71) Applicant: Cargill, Incorporated, Wayzata, MN (US)

(72) Inventors: Peter Alan Jauert, Minneapolis, MN (US); Hans H. Liao, Plymouth, MN (US); Douglas Paul Lies, Otsego, MN (US); Catherine Bradshaw Poor, Minneapolis, MN (US)

(73) Assignee: Cargill, Incorporated, Wayzata, MN (US)

(21) Appl. No.: 19/480,845

(22) PCT Filed: May 2, 2024
(86) PCT No.: PCT/US2024/027359

§ 371 Date: Nov. 3, 2025

Related U.S. Application Data
(60) Provisional application No. 63/499,990, filed on May 4, 2023.

(51) Int. Cl. C12P 7/18; C12N 9/04; C12N 9/12; C12N 9/16; C12N 9/90

(52) U.S. Cl. CPC C12P 7/18; C12N 9/0006; C12N 9/1205; C12N 9/16; C12N 9/90; C12Y 101/01009; C12Y 101/01301; C12Y 207/01017; C12Y 301/03058; C12Y 501/03001

(57) ABSTRACT

The present invention is directed to cellulytic host cells. The host cells of the invention expressing heterologous cellulases and are able to produce ethanol from cellulose. According to the invention, host cells expressing a combination of heterologous cellulases can be used to produce ethanol from cellulose. In addition, multiple host cells expressing different heterlogous cellulases can be co-cultured together and used to produce ethanol from cellulose. Furthemore, the invention demonstrates for the first time the ability of Kluveryomyces to produce ethanol from cellulose. The yeast strains and co-cultures of yeast strains of the invention can be used to produce ethanol on their own, or can also be used in combination with externally added cellulases to increase the efficiency of saccharification and fermentation processes.

### CROSS-REFERENCE TO RELATED APPLICATIONS

This application claims the benefit of U.S. Provisional Application No. 63/499,990, filed May 4, 2023, which is incorporated by reference herein in its entirety.

### REFERENCE TO A SEQUENCE LISTING SUBMITTED VIA PATENT CENTER

The content of the Sequence Listing XML file of the sequence listing named “PT-1764-WO-PCT.xml” which is 847,108 bytes in size created on Apr. 29, 2024 and electronically submitted via Patent Center herewith the application is incorporated by reference in its entirety.

### BACKGROUND

Xylitol is a low-calorie sweetener used as a food additive and sugar substitute. Commonly used in drug, dietary supplement, confectionary, and toothpaste compositions, xylitol has also been associated with anticariogenic properties when used in chewing gums. Traditional methods of xylitol production, including chemically catalyzed hydrogenation of xylose hydrolyzed from biomass extracted xylan, are both monetarily and environmentally costly. These methods require high temperatures and pressures, large amounts of water, and metal catalysts that must be mined. In contrast, fermentation processes have been used commercially at large scale to produce other organic molecules, such as ethanol, citric acid, lactic acid, and the like, and may offer a cost effective and sustainable alternative to traditional xylitol processing methods.

In the development of microorganism-based fermentation strategies for the production of xylitol, production of metabolic pathway intermediates and alternative fermentation products are important considerations. For example, metabolic pathways active in the production of xylitol may have overlap with the metabolic pathways for the production of arabitol, erythritol, ribitol, and the like. However, production of other metabolites pulls carbon away from the production of xylitol. Accordingly, provided herein are genetically modified yeast and fermentation methods for the production of xylitol, wherein the production of erythritol is also reduced or eliminated.

### SUMMARY

The present disclosure provides a genetically engineered yeast cell capable of producing xylitol, the engineered yeast cell comprising a deletion or disruption of a native gene encoding an erythrose reductase enzyme at least 60%, at least 65%, at least 70%, at least 75%, at least 80%, at least 85%, at least 90%, at least 95%, at least 98%, at least 99%, or 100% identical to at least one of SEQ ID NOs: 292 and 295. The yeast cell may be an osmotolerant yeast cell. The yeast cell may be a cell of the subphylum Ustilaginomycotina. The yeast cell may be selected form the group consisting of Trichosporonoides megachiliensis, Trychosporonoides oedocephalis, Trychosporonoides nigrescens, Pseudozyma tsukubaensis, Trigonopsis variabilis, Moniliella, Ustilaginomycetes, Trichosporon, Yarrowia lipolytica, Penicillium, Torula, Pichia, Candida, Candida magnoliae, and Aureobasidium. When the engineered cell is used in a fermentation process in the presence of dextrose, titer and/or yield of erythritol production is decreased relative to titer and/or yield of erythritol in an equivalent fermentation process using an equivalent cell in which there has been no disruption or deletion of a native gene encoding an erythrose reductase at least 60%, at least 65%, at least 70%, at least 75%, at least 80%, at least 85%, at least 90%, at least 95%, at least 98%, at least 99%, or 100% identical to at least one of SEQ ID NOs: 292 and 295.

The yeast cell may be a Moniliella pollinis cell and the gene encoding the erythrose reductase enzyme is at least 60%, at least 65%, at least 70%, at least 75%, at least 80%, at least 85%, at least 90%, at least 95%, at least 98%, at least 99%, or 100% identical to at least one of SEQ ID NOs: 290 and 293. The cell may have a deletion of at least one allele of the gene encoding the erythrose reductase enzyme. The cell may have a deletion of both alleles of the gene encoding the erythrose reductase enzyme.

The cell may additionally comprises an exogenous polynucleotide sequence encoding a xylitol-phosphate dehydrogenase (XPDH) enzyme comprising a sequence at least 60%, at least 65%, at least 70%, at least 75%, at least 80%, at least 85%, at least 90%, at least 95%, at least 98%, at least 99%, or 100% identical to at least one of SEQ ID NOs: 12-15, 28-31, and 33. The XPDH enzyme may have a sequence at least 85% identical to at least one of SEQ ID NOs: 12-15, 28-31, and 33 or to at least one of SEQ ID NOs: 14, 15, 28, or 31. The XPDH enzyme has a sequence at least 90% identical to at least one of SEQ ID NOs: 12-15, 28-31, and 33 or to at least one of SEQ ID NOs: 14, 15, 28, or 31.

The cell may additionally comprise an exogenous polynucleotide sequence encoding a xylulokinase (XKS) enzyme comprising a sequence at least 60%, at least 65%, at least 70%, at least 75%, at least 80%, at least 85%, at least 90%, at least 95%, at least 98%, at least 99%, or 100% identical to at least one of SEQ ID NOs: 188 and 189. The cell may additionally comprise an exogenous polynucleotide sequence encoding a xylitol dehydrogenase (XDH) enzyme comprising a sequence at least 60%, at least 65%, at least 70%, at least 75%, at least 80%, at least 85%, at least 90%, at least 95%, at least 98%, at least 99%, or 100% identical to at least one of SEQ ID NOs: 190, 191, and 192.

The cell may additionally comprise a genetic modification resulting in overexpression of a native enzyme with ribulose-5-phosphate epimerase (RPE) activity. The yeast cell may be a yeast cell of the genus Moniliella. The yeast cell may be a Moniliella pollinis cell and the native RPE enzyme comprises a sequence at least 60%, at least 65%, at least 70%, at least 75%, at least 80%, at least 85%, at least 90%, at least 95%, at least 98%, at least 99%, or 100% identical to at least one of SEQ ID NOs: 179 and 180. The genetic modification resulting in overexpression of a native RPE enzyme comprises addition of an exogenous polynucleotide encoding the native RPE enzyme such that the genetically engineered cell comprises at least one additional copy of a sequence encoding the RPE enzyme.

The yeast may additionally comprise a genetic modification resulting in overexpression of a native enzyme with xylitol-5-phosphate phosphatase (X5PP) activity; and/or an exogenous polynucleotide sequence encoding an enzyme with xylitol-5-phosphate phosphatase (X5PP) activity. The cell may be a Moniliella pollinis cell and the genetic modification results in overexpression of a native X5PP enzyme with a sequence at least 60%, at least 65%, at least 70%, at least 75%, at least 80%, at least 85%, at least 90%, at least 95%, at least 98%, at least 99%, or 100% identical to at least one of SEQ ID NOs: 198, 199, 200, or 221. The genetic modification comprises addition of an exogenous polynucleotide sequence encoding the native X5PP enzyme such that the genetically engineered cell comprises at least one additional copy of a sequence encoding the native X5PP enzyme. The yeast cell may comprise an exogenous polynucleotide sequence encoding an enzyme with X5PP activity and a sequence at least 60%, at least 65%, at least 70%, at least 75%, at least 80%, at least 85%, at least 90%, at least 95%, at least 98%, at least 99%, or 100% identical to at least one of SEQ ID NOs: 200, 201, 202, 203, 204, 205, 206, 207, 208, 209, 210, 211, 213, 214, 221, 222, 189, and 188. The yeast cell comprises an exogenous polynucleotide sequence encoding an enzyme with X5PP activity and a sequence at least 60%, at least 65%, at least 70%, at least 75%, at least 80%, at least 85%, at least 90%, at least 95%, at least 98%, at least 99%, or 100% identical to at least one of SEQ ID NOs: 200, 202, 203, 204, 205, 206, 210, and 213. The yeast cell comprises an exogenous polynucleotide sequence encoding an enzyme with X5PP activity and a sequence at least 60%, at least 65%, at least 70%, at least 75%, at least 80%, at least 85%, at least 90%, at least 95%, at least 98%, at least 99%, or 100% identical to at least one of SEQ ID NOs: 200, 203, 204, 206, and 213. X5PP activity in the genetically engineered yeast cell may be higher than X5PP activity in an equivalent cell lacking the genetic modification or exogenous polynucleotide sequence.

One or more of the exogenous polynucleotide sequence(s) described above may be operably linked to a heterologous or artificial promoter. The heterologous or artificial promoter may be selected from the group consisting of pyruvate kinase 1 promoter (PYK1p; SEQ ID NO: 86), 6-phosphogluconate dehydrogenase promoter (6PGDp; SEQ ID NO:130), glyceraldehyde-3-phosphate dehydrogenase promoter (TDH3p; SEQ ID NO:132), translational elongation factor 1 promoter (TEFp; SEQ ID NO:133), modified TEFp (SEQ ID NO:131), phosphoglucomutase 1 promoter (PGM1p; SEQ ID NO:134), 3-phosphoglycerate kinase promoter (PGK1p; SEQ ID NO:135), enolase promoter (ENO1p; SEQ ID NO:136), asparagine synthetase promoter (ASNSp; SEQ ID NO:137), 50S ribosomal protein L1 promoter (RPLAp; SEQ ID NO: 138), and RPL16B (SEQ ID NO:139). The promoter may be a constitutive promoter. One or more of the exogenous polynucleotide sequence(s) is integrated into the genome of the yeast cell at a locus selected from the ER1 locus, the ER3 locus, the PDC1 locus, the pyrF locus, the TRP3 locus, the gpdIIA locus, and the gpdIIB locus.

The disclosure also provides a method for producing xylitol using the engineered cells described herein, the method comprising contacting a substrate comprising dextrose with an engineered cell described herein, wherein fermentation of the substrate by the engineered cell produces xylitol. The fermentation temperature may be at or between 25 °C to 45 °C, 30 °C to 40 °C, or 32 °C to 37 °C. The volumetric oxygen uptake rate (OUR) may be between 5-80, 10-75, 15-70, 20-60, 30-50, or 40-50 mmol O₂/(L·h) The xylitol may be produced at a rate of at least 0.2, 0.3, 0.5, 0.75, or at least 1.0 g L⁻¹ h⁻¹. Xylitol production may be at least at least 20, 30, 50, 75, or 100 g/L when the fermentation is run at 35 °C for 96 hours. The concentration of dextrose may be at least 100 g/L. Rate, titer, and/or yield of erythritol production is decreased relative to an equivalent fermentation run with an equivalent yeast cell in which the native gene encoding the erythrose reductase enzyme has not been deleted or disrupted. The erythritol titer may be less than 20, 18, 15, 12, 10, 5, 1, 0.5 g/L when the fermentation is run at 35 °C for 96 hours. The erythritol production rate may be less than 0.5, 0.4, 0.3, 0.25, 0.2, 0.15, or 0.1 g L⁻¹ h⁻¹. The erythritol yield may be less than 35%, 30%, 25%, 20%, 15%, 10%, 5%, or 1%.

The disclosure also provides use of the engineered cells described herein to produce xylitol.

### BRIEF DESCRIPTION OF THE FIGURES

This patent or application contains at least one drawing executed in color. Copies of this patent or patent application publication with color drawings will be provided by the Office upon request and the payment of the necessary fee.

The drawings illustrate generally, by way of example, but not by way of limitation, various aspects discussed herein.

FIG. 1 shows the native pentose phosphate pathway (dotted lines and arrows) and the native glycolysis pathways (solid lines and arrows) in Moniliella pollinis.

FIG. 2 shows diversity in the galactitol-1-phosphate-5-dehydrogenase (G1PDH)/xylitol-phosphate dehydrogenase (XPDH) sequence space.

FIG. 3 shows the structural characteristics of the NAD or NADP binding pocket located +23 amino acids from the characteristic GXGXXG motif (SEQ ID NO:133) of XPDH enzymes.

FIG. 4 shows diversity in the ribulose-5-phosphate reductase sequence space.

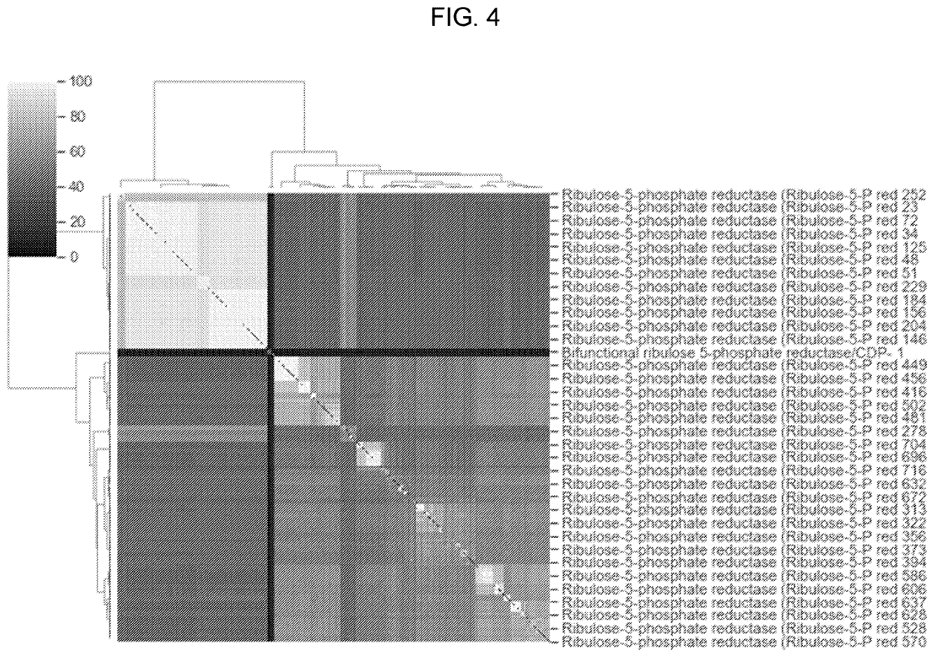

FIG. 5 shows in vitro activity of TarJ′ and XPDH enzymes as outlined in Example 3.

FIG. 6 shows erythritol, ribitol, and xylitol metabolite concentrations (g/L) at 96 hours of shake flask fermentations of strains 1-1, 1-13a-f, and 1-15a-f as outlined in Example 5. Data labels report the concentration (g/L) of xylitol.

FIG. 7 shows erythritol, ribitol, and xylitol metabolite concentrations (g/L) at 96 hours of shake flask fermentations of strains 1-1, 1-35a-d, 1-37a-d, 1-38a-f, and 1-39a-f as outlined in Example 5. Data labels report the concentration (g/L) of xylitol.

FIG. 8 shows erythritol, ribitol, and xylitol metabolite concentrations (g/L) at 96 hours of shake flask fermentations of strains 1-13c, 1-29a-e, 1-33a-e, and 1-34a-e as outlined in Example 6. Data labels report the concentration (g/L) of xylitol.

FIG. 9 shows erythritol, ribitol, arabitol, and xylitol metabolite concentrations (g/L) at 96 hours of shake flask fermentations of strains 1-13c, 1-12a-e, 1-14a-e, and 1-16a-e as outlined in Example 8. Data labels report the concentration (g/L) of xylitol (strains 1-13c, 1-14a-e, and 1-16a-e) or arabitol (strains 12a-e).

FIG. 10 shows erythritol, ribitol, and xylitol metabolite concentrations (g/L) at 96 hours of shake flask fermentations of strains-13c, 1-36a-e, and 1-40a-e as outlined in Example 9. Data labels report the concentration (g/L) of xylitol.

FIG. 11 shows erythritol, ribitol, and xylitol metabolite concentrations (g/L) at 96 hours of shake flask fermentations of strains 1-30a-e, 1-31a-e, 1-32a-e, and 1-13c as outlined in Example 10. Data labels report the concentration (g/L) of xylitol.

FIG. 12 shows a comparison of xylitol and ribitol concentrations (g/L) produced in strains with (3-7a-f and 3-8a-f) and without (1-13c and 1-15a) RPE2 overexpression.

FIG. 13 shows respective yields of xylitol, ribitol, glycerol, and erythritol for strains with (3-8a-f) and without (1-15a) RPE2 overexpression.

FIG. 14 shows xylitol and ribitol titers (g/L) for the indicated strains.

FIG. 15 shows respective yields of xylitol, ribitol, glycerol, and erythritol for the indicated strains with various configurations of RPE overexpression.

FIG. 16 shows xylitol concentrations (g/L) at 96 hours of shake flask fermentations of the indicated strains as outlined in Example 15.

FIG. 17 shows xylitol concentrations (g/L) at 96 hours of shake flask fermentations of the indicated strains as outlined in Example 15.

FIG. 18 shows xylitol rate (g/(L·h)) between 48 and 96 hours of shake flask fermentations of the indicated strains as outlined in Example 15.

FIG. 19 shows xylitol rate (g/(L·h)) between 48 and 96 hours of shake flask fermentations of the indicated strains as outlined in Example 15.

FIG. 20 shows xylitol yield (%) between 48 and 96 hours of shake flask fermentations of the indicated strains as outlined in Example 15.

FIG. 21 shows xylitol yield (%) between 48 and 96 hours of shake flask fermentations of the indicated strains as outlined in Example 15.

FIG. 22 shows erythritol, ribitol, and xylitol metabolite concentrations (g/L) at 72 hours of shake flask fermentations of strains as outlined in Example 18. Data labels report the concentration (g/L) of xylitol.

FIG. 23 shows erythritol, ribitol, and xylitol metabolite concentrations (g/L) at 72 hours of shake flask fermentations of strains as outlined in Example 18. Data labels report the concentration (g/L) of xylitol.

FIG. 24 shows erythritol, ribitol, and xylitol metabolite concentrations (g/L) at 72 hours of shake flask fermentations of strains as outlined in Example 22. Data labels report the concentration (g/L) of xylitol.

FIG. 25 shows erythritol, ribitol, and xylitol metabolite concentrations (g/L) at 72 hours of shake flask fermentations of strains as outlined in Example 22. Data labels report the concentration (g/L) of xylitol.

FIG. 26 shows erythritol, ribitol, and xylitol metabolite concentrations (g/L) at 72 hours of shake flask fermentations of strains as outlined in Example 23. Data labels report the concentration (g/L) of xylitol.

FIG. 27 shows erythritol, ribitol, and xylitol metabolite concentrations (g/L) at 72 hours of shake flask fermentations of strains as outlined in Example 23. Data labels report the concentration (g/L) of xylitol.

FIG. 28 shows erythritol, ribitol, and xylitol metabolite concentrations (g/L) at 72 hours of shake flask fermentations of strains as outlined in Example 24. Data labels report the concentration (g/L) of xylitol.

FIG. 29 shows erythritol, ribitol, and xylitol metabolite concentrations (g/L) at 72 hours of shake flask fermentations of strains as outlined in Example 24. Data labels report the concentration (g/L) of xylitol.

FIG. 30 shows erythritol, ribitol, and xylitol metabolite concentrations (g/L) at 72 hours of shake flask fermentations of strains as outlined in Example 25. Data labels report the concentration (g/L) of xylitol.

FIG. 31 shows erythritol, ribitol, and xylitol metabolite concentrations (g/L) at 72 hours of shake flask fermentations of strains as outlined in Example 27. Data labels report the concentration (g/L) of xylitol.

FIG. 32 shows erythritol, ribitol, and xylitol metabolite concentrations (g/L) at 72 hours of shake flask fermentations of strains as outlined in Example 27. Data labels report the concentration (g/L) of xylitol.

FIG. 33 shows erythritol and xylitol metabolite concentrations (g/L) at 72 hours of shake flask fermentations of strains as outlined in Example 28. Data labels report the concentration (g/L) of xylitol.

FIG. 34 shows enzyme activity for the in vitro assay described in Example 29.

FIG. 35 shows erythritol, xylitol, and glycerol metabolite concentrations (g/L) at 96 hours of shake flask fermentation of strains as outlined in Example 31. Data labels report the concentration of xylitol.

FIG. 36 shows erythritol, xylitol, and glycerol metabolite concentrations (g/L) at 96 hours of shake flask fermentation of strains as outlined in Example 32.

FIG. 37 shows erythritol, xylitol, and glycerol metabolite concentrations (g/L) at 96 hours of shake flask fermentation of strains as outlined in Example 33.

FIG. 38 shows erythritol, xylitol, and glycerol metabolite concentrations (g/L) at 96 hours of shake flask fermentations of strains as outlined in Example 34.

FIG. 39 shows erythritol, xylitol, and glycerol metabolite concentrations (g/L) at 96 hours of shake flask fermentations of strains as outlined in Example 34. Data labels report the concentration of xylitol.

FIG. 40 shows erythritol, xylitol, and glycerol metabolite concentrations (g/L) at 96 hours of shake flask fermentations of strains as outlined in Example 35. Data labels report the concentration of erythritol.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

In this document, the terms “a,” “an,” or “the” are used to include one or more than one unless the context clearly dictates otherwise. The term “or” is used to refer to a nonexclusive “or” unless otherwise indicated. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless expressly stated, ppm (parts per million), percentage, and ratios are on a by weight basis. Percentage on a by weight basis is also referred to as wt % or % (wt) below.

This disclosure relates to various recombinant cells engineered to produce xylitol. In general, the recombinant cells described herein are capable of producing xylitol and are characterized by deletion or disruption of a native gene encoding an erythrose reductase enzyme. The recombinant yeast may additionally be characterized by overexpression of a ribulose 5-phosphatase epimerase (RPE) enzyme; inclusion of an exogenous polynucleotide sequence encoding a xylitol phosphate dehydrogenase (XPDH) enzyme; and/or overexpression of a xylitol-5-phosphate phosphatase (X5PP) enzyme. The disclosure further provides fermentation methods for the production of xylitol from dextrose, with reduced erythritol production, using the genetically engineered cells described herein.

In general, recombinant cells described herein are yeast cells. As used herein, “yeast” refers to eukaryotic single celled microorganisms classified as members of the fungus kingdom. Yeast are unicellular organisms which evolved from multicellular ancestors with some species retaining multicellular characteristics such as forming strings of connected budding cells known as pseudo hyphae or false hyphae. Yeast cells may also be referred to in the art as yeast-like cells, and as used herein “yeast cell” encompasses both yeast and yeast-like cells. Suitable yeast and yeast-like host cells for modification may include, but are not limited to, Saccharomyces cerevisiae, Komagataella sp., Kluyveromyces (e.g., Kluyveromyces lactis, Kluveromyces marxianus), Yarrowia lipolytica, Issatchenkia orientalis, Pichia galeiformis, Pichia sp. YB-4149 (NRRL designation), Pichia pastoris, Candida (e.g., Candida magnoliae, Candida ethanolica), Pichia deserticola, Pichia membranifadens, Pichia fermentans, Aspergillus, Trichoderma, Myceliphthora thermophila, Moniliella (e.g., Moniliella pollinis), Pfaffia, Yamadazyma, Hansenula, Pichia kudriavzevvi, Trichosporonoides (e.g., % Trichosporonoides megachiliensis, Trychosporonoides oedocephalis, Trychosporonoides nigrescens), Pseudozyma tsukubaensis, Trigonopsis variabilis, Penicillium, and Torula. An ordinarily skilled artisan would understand the requirements for selection of a suitable yeast cell, and recombinant yeast cells of the present disclosure are not limited to those expressly recited herein. Methods for genetic engineering of yeast cells are known and described in the art and a skilled artisan would understand the methods necessary to transform and engineer a suitable yeast cell.

A suitable yeast cell may be a cell of the phylum Basidiomycota and the subphylum Ustilaginomycotina. Suitable yeast of the subphylum Ustilaginomycotina include, but are not limited to, Ustilago (e.g., U. cynodontis, U. maydis, U. sphaerogena, U. cordal, U. scitaminea, U. coicis, U. syntherismae, U. esculenta, U. neglecta, U. crus-galli, Ustilago avenae), Sporisorium (e.g., Sporisorium exsertum), Moniliella (e.g., M. pollinis, M. tomentosa, M. acetoabutans, M. fonsecae, M. madida, M. megachiliensis, M. ocedocephalis, M. nigrescens), and Pseudozyma (e.g., Pseudozyma tsukubaensis), and Trichosporonoides (e.g., Trichosporonoides megachiliensis, Trychosporonoides oedocephalis, Trychosporonoides nigrescens). Yeast of the subphylum Ustilaginomycotina have been known and described in the art as potential production organisms for valuable chemicals such as itaconate, malate, succinate, mannitol, and erythritol and other valuable biotechnological applications. See, for example, Geiser et al. (Prospecting the biodiversity of the fungal family Ustilaginacceae for the production of value-added chemicals,” Fungal Biol Biotechnol, 2014, 1:2), Feldbrugge et al., (“The biotechnological use and potential of plant pathogenic smut fungi,” Appl Microbiol Biotechnol, 2013, 97 (8): 3253-65), Guevarra et al., (“Accumulation of itaconic, 2-hydroxyparaconic, itatartaric, and malic acids by strains of the genus Ustilago, Agric. Biol. Chem., 1990, 54 (9), 2353-2358), and Moon et al., (“Biotechnological production of erythritol and its applications,” Appl Microbiol Biotechnol, 2010, 86:1017-1025).

A suitable yeast cell will have an active pentose phosphate pathway that produces ribulose-5-phosphate. As used herein “active pentose phosphate pathway” refers to expression of one or more functional enzymes which, together, convert glucose-6-phosphate, NADP⁺ or NAD+, and water to NADPH or NADH, CO2, and ribulose-5-phosphate. Continuing in a non-oxidative phase, the pathway may also produce other pentose (i.e., 5-carbon) sugars. For example, the pentose phosphate pathway may produce ribulose-5-phosphate, ribose-5-phosphate, xylulose-5-phosphate, fructose 6-phosphate, combinations thereof, and the like, depending on the enzymatic activities present. The active pentose phosphate pathway may be native to the yeast cell or it may be introduced into the yeast cell by genetic engineering.

The yeast cell may be an osmotolerant yeast cell. As used herein, “osmotolerant” refers to a yeast capable of growth and reproduction under conditions of high osmolarity, such as at least 10% (w/v), at least 20% (w/v), at least 30% (w/v), at least 40% (w/v), at least 50% (w/v), or at least 60% (w/v) glucose and/or at least 6% (w/v), at least 10% (w/v), at least 12% (w/v), at least 13% (w/v), at least 15% (w/v) sodium chloride. Species and strains of osmotolerant yeast are known and described in the art, including many species of yeast used in industrial fermentation processes. Likewise, methods for assaying yeast osmotolerance are known and described in the art. See, for example, Tiwari, S., et al., (“Nectar yeast community of tropical flowering plants and assessment of their osmotolerance and xylitol-producing potential,” Current Microbiology, 2022, 79:28).

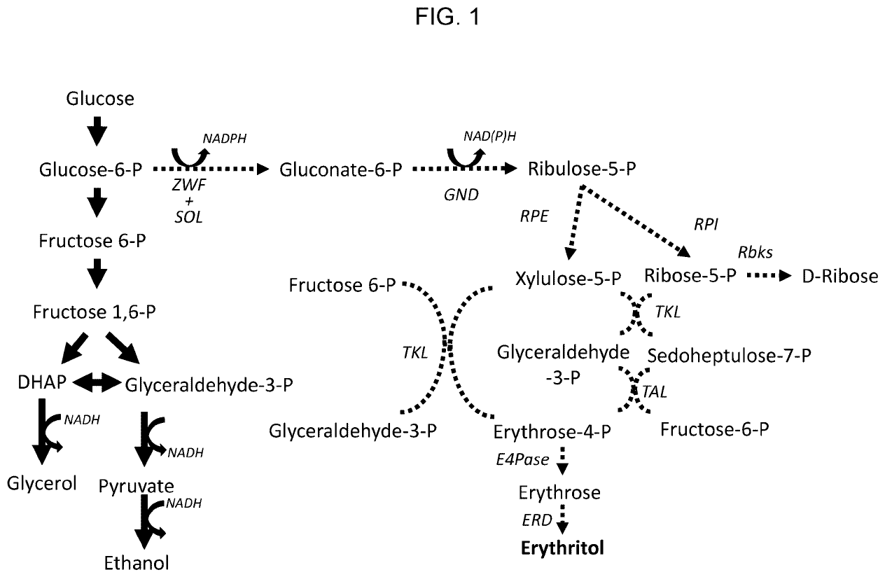

The recombinant yeast cell may be a recombinant Moniliella cell, for example, a Moniliella pollinis cell. FIG. 1 shows the predicted native pentose phosphate and glycolysis pathways in Moniliella pollinis. Moniliella has previously been used in the fermentation production of erythritol and methods for genetically modifying and fermenting Moniliella are known and described in the art. See, for example, Li et al. (“Methods for genetic transformation of filamentous fungi,” 2017, Microb Cell Fact, 16:168).

Various plasmids and methods for transformation of Moniliella are also described in the Examples below. For example, Moniliella may be transformed using a bipartite polynucleotide sequence in which, following recombination, the exogenous polynucleotide of interest is integrated at the specified locus and the selection marker is expressible within the cell. Suitable selection markers are known and used in the art. The selectable marker may include, but is not limited to, amdS (for example broken into a 3′ portion, SEQ ID NO: 167, and a 5′ portion, SEQ ID NO:174), G418 resistance gene (for example broken into a 3′ portion, SEQ ID NO:172, and a 5′ portion, SEQ ID NO:175), zeocin resistance gene (for example broken into a 3′ portion, SEQ ID NO: 168, and a 5′ portion, SEQ ID NO:169), nourseothricin N-acetyl transferase (NAT) (for example broken into a 3′ portion, SEQ ID NO: 171, and a 5′ portion, SEQ ID NO:170), and invertase gene (SUC2) (for example a 3′ portion of SEQ ID NO: 173 and a 5′ portion of SEQ ID NO: 176).

The recombinant cells described herein include one or more exogenous polynucleotide sequences encoding one or more polypeptides that, when expressed, improve the fermentation of glucose to xylitol by the recombinant cells.

The terms “glucose” and “dextrose” are used interchangeably herein and refer to D-glucose except where expressly indicated otherwise.

As used herein, “exogenous” refers to genetic material or an expression product thereof that originates from outside of the host organism. For example, the exogenous genetic material or expression product thereof can be a modified form of genetic material native to the host organism, it can be derived from another organism, it can be a modified form of a component derived from another organism, or it can be a synthetically derived component. For example, a K. lactis invertase gene is exogenous when introduced into S. cerevisiae.

As used herein, “native” refers to genetic material or an expression product thereof that is found, apart from individual-to-individual mutations which do not affect function or expression, within the genome of wild-type cells of the host cell. For the purposes of this application, the Moniliella pollinis cell “Moniliella tomentosa var pollinis TCV364” described in U.S. Pat. No. 6,440,712, which is incorporated herein by reference in its entirety, and deposited under the Budapest Treaty at BCCM/MUCL (Belgian Coordinated Collections of Micro-organisms/Mycothèque de l′Université Catholique de Louvain by Eridania Béghin Say, Vilvoorde R&D Centre, Havenstraat 84, B-1800 Vilvoorde) on Mar. 28, 1997 under number MUCL40385, is considered the wild-type Moniliella pollinis cell.

As used herein, the terms “polypeptide” and “peptide” are used interchangeably and refer to the collective primary, secondary, tertiary, and quaternary amino acid sequences and structure necessary to give the recited macromolecule its function and properties. As used herein, “enzyme” or “biosynthetic pathway enzyme” refer to a protein that catalyzes a chemical reaction. The recitation of any particular enzyme, either independently or as part of a biosynthetic pathway is understood to include the co-factors, co-enzymes, and metals necessary for the enzyme to properly function. A summary of the amino acids and their three and one letter symbols as understood in the art is presented in Table 1. The amino acid name, three letter symbol, and one letter symbol are used interchangeably herein.

TABLE 1
Amino Acid three and one letter symbols

| | Three | One |
|---|---|---|
| | letter | letter |
| Amino Acid | symbol | symbol |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Variants or sequences having substantial identity or homology with the polypeptides described herein can be utilized in the practice of the disclosed recombinant cells, compositions, and methods. Such sequences can be referred to as variants or modified sequences. That is, a polypeptide sequence can be modified yet still retain the ability to exhibit the desired activity. Generally, the variant or modified sequence may include greater than about 45%, 50%, 55%, 60%, 65%, 70%, 75%, 80%, 85%, 90%, or 95% sequence identity with the wild-type, naturally occurring polypeptide sequence, or with a variant polypeptide as described herein.

As used herein, the phrases “% sequence identity,” “% identity,” and “percent identity,” are used interchangeably and refer to the percentage of residue matches between at least two amino acid sequences or at least two nucleic acid sequences aligned using a standardized algorithm. Methods of amino acid and nucleic acid sequence alignment are well-known. Sequence alignment and generation of sequence identity include global alignments and local alignments which are carried out using computational approaches. An alignment can be performed using BLAST (National Center for Biological Information (NCBI) Basic Local Alignment Search Tool) version 2.2.31 software with default parameters. Amino acid % sequence identity between amino acid sequences can be determined using standard protein BLAST with the following default parameters: Max target sequences: 100; Short queries: Automatically adjust parameters for short input sequences; Expect threshold: 10; Word size: 6; Max matches in a query range: 0; Matrix: BLOSUM62; Gap Costs: (Existence: 11, Extension: 1); Compositional adjustments: Conditional compositional score matrix adjustment; Filter: none selected; Mask: none selected. Nucleic acid % sequence identity between nucleic acid sequences can be determined using standard nucleotide BLAST with the following default parameters: Max target sequences: 100; Short queries: Automatically adjust parameters for short input sequences; Expect threshold: 10; Word size: 28; Max matches in a query range: 0; Match/Mismatch Scores: 1, −2; Gap costs: Linear; Filter: Low complexity regions; Mask: Mask for lookup table only. A sequence having an identity score of XX % (for example, 80%) with regard to a reference sequence using the NCBI BLAST version 2.2.31 algorithm with default parameters is considered to be at least XX % identical or, equivalently, have XX % sequence identity to the reference sequence.

Polypeptide or polynucleotide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The polypeptides disclosed herein may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant,” “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule.

The amino acid sequences of the polypeptide variants, mutants, derivatives, or fragments as contemplated herein may include conservative amino acid substitutions relative to a reference amino acid sequence. For example, a variant, mutant, derivative, or fragment polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge and/or hydrophobicity of the molecule at the site of the substitution, and/or (c) the bulk of the side chain.

As used herein, terms “polynucleotide,” “polynucleotide sequence,” and “nucleic acid sequence,” and “nucleic acid,” are used interchangeably and refer to a sequence of nucleotides or any fragment thereof. These phrases also refer to DNA or RNA of natural or synthetic origin, which may be single-stranded or double-stranded and may represent the sense or the antisense strand. The DNA polynucleotides may be a cDNA (e.g., coding DNA) or a genomic DNA sequence (e.g., including both introns and exons).

A polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

Those of skill in the art understand the degeneracy of the genetic code and that a variety of polynucleotides can encode the same polypeptide. In some aspects, the polynucleotides (e.g., polynucleotides encoding an erythrose reductase polypeptide) may be codon-optimized for expression in a particular cell including, without limitation, a plant cell, bacterial cell, fungal cell, or animal cell. While polypeptides encoded by polynucleotide sequences found in various species are disclosed herein any polynucleotide sequences may be used which encodes a desired form of the polypeptides described herein. Thus, non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

The recombinant cells described herein may include deletions or disruptions in one or more native genes. The phase “deletion or disruption” refers to the status of a native gene in the recombinant cell that has either a completely eliminated coding region (deletion) or a modification of the gene, its promoter, or its terminator (such as by a deletion, insertion, or mutation) so that the gene no longer produces an active expression product, produces severely reduced quantities of the expression product (e.g., at least a 75% reduction or at least a 90% reduction) or produces an expression product with severely reduced activity (e.g., at least 75% reduced or at least 90% reduced). The deletion or disruption can be achieved by genetic engineering methods, forced evolution, mutagenesis, RNA interference (RNAi), and/or selection and screening. Deletion or disruption of a native host cell gene can be coupled to the incorporation of one or more polynucleotide sequences (e.g., an exogenous or native polynucleotide sequence) into the host cell at the locus of the host cell gene to be deleted or disrupted. The polynucleotide sequence to be inserted may be designed to replace all or a portion of the host cell gene to be deleted or disrupted. The polynucleotide sequence may encode for a gene product of interest, for example, a polypeptide, an enzyme, and the like. The deletion or disruption can also be accomplished using a deletion construct that does not contain a polynucleotide sequence to be integrated. Other methods for gene disruption or deletion are known and described in the art.

The recombinant cells described herein have a deletion or disruption in one or more native genes encoding an enzyme involved in erythritol fermentation or consumption. Deletion or disruption of one or more of these biosynthetic pathway enzymes decreases the ability of the recombinant cell to produce erythritol and may, depending on the deletion or disruption, increasing carbon flux into the fermentation pathway for the production of xylitol.

The recombinant cells described herein include a deletion or disruption of a native erythrose reductase (ER) gene. The native ER gene encodes an enzyme that has erythrose reductase activity. As used herein “erythrose reductase activity” and “ER activity” are used interchangeably and refer to enzymes that catalyze the reversible conversion of erythrose or erythrose-4-phosphate to erythritol or erythritol-4-phosphate using a nicotinamide adenine dinucleotide (phosphate) (hydrogen) (NAD (P) (H)) cofactor. In the art, enzymes that catalyze the reversible conversion of erythrose-4-phosphate to erythritol-4-phosphate using a nicotinamide adenine dinucleotide (phosphate) (hydrogen) (NAD (P) (H)) cofactor may also be described as erythrose or erythritol phosphate dehydrogenases. When the host cell contains multiple ER genes, it is preferred to delete or disrupt at least one of them. When the host cell contains multiple alleles of a given ER gene, it is preferred to delete or disrupt one allele or both alleles of the given ER gene.

As used herein, “NAD (P) H” refers to nicotinamide adenine dinucleotide (phosphate) hydrogen and is inclusive of both NADH and NADPH. As is understood in the art, inclusion of the phosphate (or “P” abbreviation) in parentheses indicates that the phosphate may be absent or present and the name and abbreviation are inclusive of both. Similarly, “NAD (H)” or “NADP (H)” refers to both the reduced and oxidized forms of the cofactor.

When the recombinant cell is a Moniliella pollinis cell, the recombinant cell may comprise a deletion or disruption of an ER gene encoding an amino acid sequence at least 60%, at least 65%, at least 70%, at least 75%, at least 80%, at least 85%, at least 90%, at least 95%, at least 98%, at least 99%, or 100% identical to SEQ ID NO:295. When the recombinant cell is a Moniliella pollinis cell, the recombinant cell may comprise a deletion or disruption of an ER gene with a nucleotide sequence at least 60%, at least 65%, at least 70%, at least 75%, at least 80%, at least 85%, at least 90%, at least 95%, at least 98%, at least 99%, or 100% identical to SEQ ID NO: 293.

When the recombinant cell is a Moniliella pollinis cell, the recombinant cell may comprise a deletion or disruption of an ER gene encoding an amino acid sequence at least 60%, at least 65%, at least 70%, at least 75%, at least 80%, at least 85%, at least 90%, at least 95%, at least 98%, at least 99%, or 100% identical to SEQ ID NO:292. When the recombinant cell is a Moniliella pollinis cell, the recombinant cell may comprise a deletion or disruption of an ER gene with a nucleotide sequence at least 60%, at least 65%, at least 70%, at least 75%, at least 80%, at least 85%, at least 90%, at least 95%, at least 98%, at least 99%, or 100% identical to SEQ ID NO: 290.

The recombinant cells described herein may include one or more genetic modifications in which an exogenous nucleic acid is integrated into the genome of the host cell. One of skill in the art know how to select suitable loci in a yeast genome for integration of the exogenous nucleic acid. Suitable integration loci may include, but are not limited to, the PDC1, GPD1, CYB2A, CYB2B, g4240, YMR226, MDHB, ATO2, Adh9091, Adh1202, ADE2, ADH2556, GAL6, MDH1, SCW11, ER1, ER3, pyrF, TRP3, gpdIIA, and gpdIIB loci. For example, in a M. pollinis host cells, suitable interaction loci may include, but are not limited to, the ER1 locus (defined as the locus flanked by SEQ ID NO:85 and SEQ ID NO:162), the ER3 locus (defined as the locus flanked by SEQ ID NO: 155 and SEQ ID NO: 165), the PDC1 locus (defined as the locus flanked by SEQ ID NO: 152 and SEQ ID NO: 164), the pyrF locus (defined as the locus flanked by SEQ ID NO: 153 and SEQ ID NO:163), the TRP3 locus (defined as the locus flanked by SEQ ID NO: 156 and SEQ ID NO:159), the gpdIIA locus (defined as the locus flanked by SEQ ID NO:157 and SEQ ID NO: 161); and the gpdIIB locus (defined as the locus flanked by SEQ ID NO: 158 and SEQ ID NO: 166). The exogenous nucleic acid may also be integrated in an intergenic region or other location in the host cell genome not specifically specified herein. Other suitable integration loci may be determined by one of skill in the art. Furthermore, one of skill in the art would recognize how to use sequences to design primers to verify correct gene integration at the chosen locus.

The recombinant cell may have one or more copies of a given exogenous nucleic acid sequence integrated in a host chromosome(s) and replicated together with the chromosome(s) into which it has been integrated. For example, the yeast cell may be transformed with nucleic acid construct including a polynucleotide sequence encoding for a polypeptide described herein and the polynucleotide sequence encoding for the polypeptide may be integrated in one or more copies in a host chromosome(s). The recombinant cell may include multiple copies (two or more) of a given polynucleotide sequence encoding a polypeptide described herein. The recombinant cell may have one, two, three, four, five, six, seven, eight, nine, ten, or more copies of a polynucleotide sequence encoding a polypeptide described herein integrated into the genome. The multiple copies of said polynucleotide sequence may all be incorporated at a single locus or may be incorporated at multiple loci.

The final step in the xylitol pathway, from xylitol 5-phosphate to xylitol, requires a phosphatase enzyme. The Saccharomyces cerevisiae PYP1 (polyol phosphatase 1) gene encodes a sugar alcohol phosphatase that hydrolyzes sorbitol-6-phosphate, ribitol-5-phosphate, and (D)-glycerol-3-phosphase (Xu et al., “Discovery and functional characterization of a yeast sugar alcohol phosphatase,” ACS Chem. Biol., 13, 2018, 3011-3020). PYP1 is a member of the haloacid dehalogenase (HAD)-like hydrolase superfamily (Kuznetsova, et a., “Functional diversity of haloacid dehalogenase superfamily phosphatases from Saccharomyces cerevisiae,” J. Biol. Chem., 2015, 290, 18678-18698) and belongs to the enzyme class of sorbitol-6-phosphatases (Enzyme Commission (EC) 3.1.3.50). As xylitol 5-phosphate is a similar molecule to the known substrates of PYP1 it is demonstrated herein that one or more PYP-like enzymes or PYP orthologs have xylitol-5-phosphate phosphatase activity and can be used to increase xylitol production in the recombinant cells described herein. E. coli HxpA (hexitol phosphatase A) is a HAD-like enzyme belonging to EC 3.1.3.50 with a similar substrate profile to PYP1 (Kuznetsova et al., “Genome wide analysis of substrate specificities of the Escherichia coli haloacid dehalogenase-like phosphate family,” 2006, J. Biol. Chem., 281, 36149-36161). Accordingly, it is also demonstrated herein that one or more HAD-like hydrolase enzymes or HAD-like hydrolase orthologs have xylitol-5-phosphate phosphatase activity and can be used to increase xylitol production in the recombinant cells describe here.

The recombinant cells described herein are capable of producing xylitol, have a deletion or disruption of an ER gene, and may be characterized by overexpression of a native enzyme with xylitol-5-phosphate phosphatase (X5PP) activity and/or include an exogenous polynucleotide sequence encoding a native or exogenous enzyme with xylitol-5-phosphate phosphatase (X5PP) activity. In general, the recombinant cell(s) including overexpression of an X5PP enzyme or expressing an exogenous X5PP enzyme produce more xylitol than an equivalent cell lacking the exogenous X5PP enzyme or lacking overexpression of the X5PP enzyme. The enzyme may be any suitable enzyme with X5PP activity. As used herein, “X5PP enzyme” and “X5PP” are interchangeable and refer to an enzyme with X5PP activity. Herein, “xylitol-5-phosphate phosphatase activity” and “X5PP activity” are used interchangeably and refer to the ability to catalyze the conversion of xylitol-5-phosphate to xylitol and phosphate. Suitable X5PP enzymes may include a divalent metal cation, for example, Mg²⁺, Mn²⁺, or Co²⁺. Suitable enzymes with X5PP activity may include, but are not limited to, those classified under EC 3.1.3.50, for example, sugar alcohol phosphatases and HAD-like hydrolases. Polynucleotides encoding X5PP enzymes may be derived from any suitable source. For example, a polynucleotide encoding an X5PP enzyme may be derived from Moniliella pollinis, Saccharomyces cerevisiae, Lachancea dasiensis, Tetrapisispora blattae, Saccharomyces pastorianus, Kazachstania africana, Podospora comata, Geotrichum candidum, Ogattaea haglerorum, Debaryomyces fabryi, Monilinia fructicola, Nadsonia fulvescens var. elongata DSM 6958, Escherichia coli, Wickerhamomyces ciferrii, Bacillus amyloliquefaciens, and the like. The X5PP enzyme may be a polypeptide with an amino acid sequence at least 50%, at least 60%, at least 70%, at least 80%, at least 85%, at least 90%, at least 95%, at least 97%, at least 99%, or 100% identical to the amino acid sequence of at least one of SEQ ID NOs: 200, 201, 202, 203, 204, 205, 206, 207, 208, 209, 210, 211, 213, 214, 221, 222, 189, and 188. The X5PP enzyme may be a polypeptide with an amino acid sequence at least 50%, at least 60%, at least 70%, at least 80%, at least 85%, at least 90%, at least 95%, at least 97%, at least 99%, or 100% identical to the amino acid sequence of at least one of SEQ ID NOs: 200, 202, 203, 204, 205, 206, 210, and 213. The X5PP enzyme may be a polypeptide with an amino acid sequence at least 50%, at least 60%, at least 70%, at least 80%, at least 85%, at least 90%, at least 95%, at least 97%, at least 99%, or 100% identical to the amino acid sequence of at least one of SEQ ID NOs: 200, 203, 204, 206, and 213.

The recombinant cell has a deletion or disruption of an ER gene and may comprise an exogenous polynucleotide sequence that is or may be derived from a Moniliella pollinis gene encoding the amino acid sequence of SEQ ID NO:200. The exogenous polynucleotide sequence may encode an amino acid sequence at least 60%, at least 70%, at least 80%, at least 85%, at least 90%, at least 95%, at least 97%, or at least 99%, identical to SEQ ID NO:200.

The recombinant cell has a deletion or disruption of an ER gene and may comprise an exogenous polynucleotide sequence that is or may be derived from a Saccharomyces cerevisiae gene encoding the amino acid sequence of SEQ ID NO:201. The exogenous polynucleotide sequence may encode an amino acid sequence at least 60%, at least 70%, at least 80%, at least 85%, at least 90%, at least 95%, at least 97%, or at least 99%, identical to SEQ ID NO:201.

The recombinant cell has a deletion or disruption of an ER gene and may comprise an exogenous polynucleotide sequence that is or may be derived from a Lachancea dasiensis gene encoding the amino acid sequence of SEQ ID NO:202. The exogenous polynucleotide sequence may encode an amino acid sequence at least 60%, at least 70%, at least 80%, at least 85%, at least 90%, at least 95%, at least 97%, or at least 99%, identical to SEQ ID NO:202.

The recombinant cell has a deletion or disruption of an ER gene and may comprise an exogenous polynucleotide sequence that is or may be derived from a Tetrapisispora blattae gene encoding the amino acid sequence of SEQ ID NO:203. The exogenous polynucleotide sequence may encode an amino acid sequence at least 60%, at least 70%, at least 80%, at least 85%, at least 90%, at least 95%, at least 97%, or at least 99%, identical to SEQ ID NO:203.

The recombinant cell has a deletion or disruption of an ER gene and may comprise an exogenous polynucleotide sequence that is or may be derived from a Saccharomyces pastorianus gene encoding the amino acid sequence of SEQ ID NO:204. The exogenous polynucleotide sequence may encode an amino acid sequence at least 60%, at least 70%, at least 80%, at least 85%, at least 90%, at least 95%, at least 97%, or at least 99%, identical to SEQ ID NO:204.

The recombinant cell has a deletion or disruption of an ER gene and may comprise an exogenous polynucleotide sequence that is or may be derived from a Kazachstania africana gene encoding the amino acid sequence of SEQ ID NO:205. The exogenous polynucleotide sequence may encode an amino acid sequence at least 60%, at least 70%, at least 80%, at least 85%, at least 90%, at least 95%, at least 97%, or at least 99%, identical to SEQ ID NO:205.

The recombinant cell has a deletion or disruption of an ER gene and may comprise an exogenous polynucleotide sequence that is or may be derived from a Podospora comata gene encoding the amino acid sequence of SEQ ID NO:206. The exogenous polynucleotide sequence may encode an amino acid sequence at least 60%, at least 70%, at least 80%, at least 85%, at least 90%, at least 95%, at least 97%, or at least 99%, identical to SEQ ID NO:206.

The recombinant cell has a deletion or disruption of an ER gene and may comprise an exogenous polynucleotide sequence that is or may be derived from a Geotrichum candidum gene encoding the amino acid sequence of SEQ ID NO:207. The exogenous polynucleotide sequence may encode an amino acid sequence at least 60%, at least 70%, at least 80%, at least 85%, at least 90%, at least 95%, at least 97%, or at least 99%, identical to SEQ ID NO:207.

The recombinant cell has a deletion or disruption of an ER gene and may comprise an exogenous polynucleotide sequence that is or may be derived from a Ogattaea haglerorum gene encoding the amino acid sequence of SEQ ID NO:208. The exogenous polynucleotide sequence may encode an amino acid sequence at least 60%, at least 70%, at least 80%, at least 85%, at least 90%, at least 95%, at least 97%, or at least 99%, identical to SEQ ID NO:208.

The recombinant cell has a deletion or disruption of an ER gene and may comprise an exogenous polynucleotide sequence that is or may be derived from a Debaryomyces fabryi gene encoding the amino acid sequence of SEQ ID NO:209. The exogenous polynucleotide sequence may encode an amino acid sequence at least 60%, at least 70%, at least 80%, at least 85%, at least 90%, at least 95%, at least 97%, or at least 99%, identical to SEQ ID NO:209.

The recombinant cell has a deletion or disruption of an ER gene and may comprise an exogenous polynucleotide sequence that is or may be derived from a Monilinia fructicola gene encoding the amino acid sequence of SEQ ID NO:210. The exogenous polynucleotide sequence may encode an amino acid sequence at least 60%, at least 70%, at least 80%, at least 85%, at least 90%, at least 95%, at least 97%, or at least 99%, identical to SEQ ID NO:210.

The recombinant cell has a deletion or disruption of an ER gene and may comprise an exogenous polynucleotide sequence that is or may be derived from a Nadsonia fulvescens var. elongata DSM 6958 gene encoding the amino acid sequence of SEQ ID NO:211. The exogenous polynucleotide sequence may encode an amino acid sequence at least 60%, at least 70%, at least 80%, at least 85%, at least 90%, at least 95%, at least 97%, or at least 99%, identical to SEQ ID NO: 211.

The recombinant cell has a deletion or disruption of an ER gene and may comprise an exogenous polynucleotide sequence that is or may be derived from an Escherichia coli gene encoding the amino acid sequence of SEQ ID NO:213. The exogenous polynucleotide sequence may encode an amino acid sequence at least 60%, at least 70%, at least 80%, at least 85%, at least 90%, at least 95%, at least 97%, or at least 99%, identical to SEQ ID NO:213.

The recombinant cell has a deletion or disruption of an ER gene and may comprise an exogenous polynucleotide sequence that is or may be derived from a Wickerhamomyces ciferrii gene encoding the amino acid sequence of SEQ ID NO:214. The exogenous polynucleotide sequence may encode an amino acid sequence at least 60%, at least 70%, at least 80%, at least 85%, at least 90%, at least 95%, at least 97%, or at least 99%, identical to SEQ ID NO:214.

The recombinant cell has a deletion or disruption of an ER gene and may comprise an exogenous polynucleotide sequence that is or may be derived from a Moniliella pollinis gene encoding the amino acid sequence of SEQ ID NO:221. The exogenous polynucleotide sequence may encode an amino acid sequence at least 60%, at least 70%, at least 80%, at least 85%, at least 90%, at least 95%, at least 97%, or at least 99%, identical to SEQ ID NO:221.

The recombinant cell has a deletion or disruption of an ER gene and may comprise an exogenous polynucleotide sequence that is or may be derived from a Bacillus amyloliquefaciens gene encoding the amino acid sequence of SEQ ID NO:222. The exogenous polynucleotide sequence may encode an amino acid sequence at least 60%, at least 70%, at least 80%, at least 85%, at least 90%, at least 95%, at least 97%, or at least 99%, identical to SEQ ID NO:222.

The recombinant cell has a deletion or disruption of an ER gene and may comprise an exogenous polynucleotide sequence that is or may be derived from a Saccharomyces cerevisiae DOG2 gene encoding the amino acid sequence of SEQ ID NO: 189. The exogenous polynucleotide sequence may encode an amino acid sequence at least 60%, at least 70%, at least 80%, at least 85%, at least 90%, at least 95%, at least 97%, or at least 99%, identical to SEQ ID NO: 189.

The recombinant cell has a deletion or disruption of an ER gene and may comprise an exogenous polynucleotide sequence that is or may be derived from a Saccharomyces cerevisiae DOG1 gene encoding the amino acid sequence of SEQ ID NO: 188. The exogenous polynucleotide sequence may encode an amino acid sequence at least 60%, at least 70%, at least 80%, at least 85%, at least 90%, at least 95%, at least 97%, or at least 99%, identical to SEQ ID NO:188.

The enzyme with X5PP activity may be native to the host cell. For example, when the host organism is M. pollinis, the X5PP enzyme may be an enzyme with a sequence at least 50%, at least 60%, at least 70%, at least 80%, at least 85%, at least 90%, at least 95%, at least 97%, or at least 99% identical to at least one of SEQ ID NOs: 198, 199, 200, or 221. The recombinant cell may comprise an exogenous polynucleotide encoding an X5PP enzyme with at least 50%, at least 60%, at least 70%, at least 80%, at least 85%, at least 90%, at least 95%, at least 97%, or at least 99% sequence identity to at least one of SEQ ID NOs: 198, 199, 200, or 221. The recombinant cell may include a genetic modification that increases expression of an X5PP enzyme at least 60%, at least 65%, at least 70%, at least 75%, at least 80%, at least 85%, at least 90%, at least 95%, at least 98%, at least 99%, or 100% identical to at least one of SEQ ID NOs: 198, 199, 200, or 221. The genetic modification may include, but is not limited to, insertion of additional copies of a nucleic acid encoding the native X5PP enzyme into the cell (e.g., integration of additional copies of the X5PP encoding polynucleotide into it non-native locus in the cell), insertion of a constitutive promoter upstream of the coding region of the native X5PP enzyme encoding gene in the genome of the host cell, and/or modification of the existing promoter upstream of the coding region of the native X5PP enzyme encoding gene in the genome of the host cell. One of skill in the art will recognize that expression of a native X5PP enzyme encoding gene may be increased by a number of methods known in the art and will be able to select and apply such methods as appropriate.

As used herein, “overexpression” refers to an expression level of a polypeptide that is higher than the expression level of the same polypeptide in the absence of a genetic modification or exogenous polynucleotide encoding said polypeptide in an equivalent cell.

The recombinant cells described herein capable of producing xylitol and having a deletion or disruption of an ER gene may also be characterized by overexpression of a ribulose 5-phosphate epimerase (RPE enzyme). The recombinant cells described herein capable of producing xylitol and having a deletion or disruption of an ER gene may also include an exogenous polynucleotide sequence encoding an X5PP enzyme and/or overexpress a native X5PP enzyme, and/or may include an exogenous polynucleotide encoding a native or exogenous RPE enzyme or may have a genetic modification resulting in overexpression of a native RPE enzyme, as described herein. In general, the recombinant cell(s) including overexpression of the RPE enzyme produce more xylitol than an equivalent cell lacking the RPE enzyme or lacking overexpression of the RPE enzyme.

The recombinant cells described herein are capable of producing xylitol, have a deletion or disruption of an ER gene, and may include an exogenous polynucleotide encoding a native or exogenous RPE enzyme or may have a genetic modification resulting in overexpression of a native RPE enzyme. The RPE enzyme may be any suitable enzyme with ribulose 5-phosphate epimerase activity. As used herein, “ribulose 5-phosphate epimerase activity” and “RPE activity” are used interchangeably and refer to the ability to catalyze the conversion of ribulose-5-phosphate to xylulose-5-phosphate. The enzyme with RPE activity may be native to the host cell or the RPE enzyme may be an exogenous RPE enzyme. For example, when the host organism is M. pollinis, the RPE enzyme may be an enzyme with a sequence at least 50%, at least 60%, at least 70%, at least 80%, at least 85%, at least 90%, at least 95%, at least 97%, or at least 99% identical to at least one of SEQ ID NOs: 179 and 180. The recombinant cell may comprise an exogenous polynucleotide encoding an RPE enzyme with at least 50%, at least 60%, at least 70%, at least 80%, at least 85%, at least 90%, at least 95%, at least 97%, or at least 99% sequence identity to at least one of SEQ ID NOs: 179 and 180. The recombinant cell may include a genetic modification that increases expression of an RPE enzyme at least 60%, at least 65%, at least 70%, at least 75%, at least 80%, at least 85%, at least 90%, at least 95%, at least 98%, at least 99%, or 100% identical to at least one of SEQ ID NOs: 179 and 180. The genetic modification may include, but is not limited to, insertion of additional copies of a nucleic acid encoding the native RPE into the cell, insertion of a constitutive promoter upstream of the coding region of the native RPE gene in the genome of the host cell, and/or modification of the existing promoter upstream of the coding region of the native RPE gene in the genome of the host cell. One of skill in the art will recognize that expression of a native RPE gene may be increased by a number of methods known in the art and will be able to select and apply such methods as appropriate.

The recombinant cells described herein are capable of producing xylitol, has a deletion or disruption of an ER gene, and may include an exogenous polynucleotide sequence encoding a xylitol-phosphate dehydrogenase (XPDH) enzyme. The exogenous polynucleotide sequence may be an exogenous xylitol-phosphate dehydrogenase (XPDH) gene. A recombinant cell described herein capable of producing xylitol has a deletion or disruption of an ER gene and may include an exogenous polynucleotide sequence encoding an X5PP enzyme and/or overexpress a native X5PP enzyme, may include an exogenous polynucleotide sequence encoding an XPDH enzyme, and/or may include an exogenous polynucleotide encoding a native or exogenous RPE enzyme or may have a genetic modification resulting in overexpression of a native RPE enzyme, as described herein.

A “xylitol-phosphate dehydrogenase gene” and an “XPDH gene” are used interchangeably herein and refer to any gene or polynucleotide that encodes a polypeptide with xylitol-phosphate dehydrogenase activity. As used herein, “xylitol-phosphate dehydrogenase activity” refer to the ability to catalyze the conversion of xylulose-5-phosphate and NADPH or NADH to xylitol 5-phosphate and NADP⁺ or NAD⁺. The XPDH gene may be derived from any suitable source. For example, the XPDH gene may be derived from Clostridium difficile, Lactobacillus rhamnosus, Bacillus halodurans, Alkalihalobacillus ligniniphilus, Jeotgalibacillus soli, Heyndrickxia sporothermodurans, Clostridium fungisolvens, or Neobacillus cucumis. The XPDH gene may encode an amino acid at least 50%, at least 60%, at least 70%, at least 80%, at least 85%, at least 90%, at least 95%, at least 97%, at least 99%, or 100% sequence identity to the amino acid sequence of at least one of SEQ ID NOs: 12-15, 28-32, or 33. The XPDH gene may encode an amino acid at least 50%, at least 60%, at least 70%, at least 80%, at least 85%, at least 90%, at least 95%, at least 97%, at least 99%, or 100% sequence identity to the amino acid sequence of at least one of SEQ ID NOs: 14, 15, 28, or 31.

The recombinant cell may have a deletion or disruption of an ER gene and include an exogenous polynucleotide that is, or may be derived from, a Clostridium difficile gene encoding the amino acid of SEQ ID NO:12. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 12.

The recombinant cell may have a deletion or disruption of an ER gene and include an exogenous polynucleotide that is, or may be derived from, a Clostridium difficile gene encoding the amino acid of SEQ ID NO:13. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 13.

The recombinant cell may have a deletion or disruption of an ER gene and include an exogenous polynucleotide that is, or may be derived from, a Lactobacillus rhamnosus gene encoding the amino acid of SEQ ID NO: 14. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 14.

The recombinant cell may have a deletion or disruption of an ER gene and include an exogenous polynucleotide that is, or may be derived from, a Bacillus halodurans gene encoding the amino acid of SEQ ID NO:15. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 15.

The recombinant cell may have a deletion or disruption of an ER gene and include an exogenous polynucleotide that is, or may be derived from, a Alkalihalobacillus ligniniphilus gene encoding the amino acid of SEQ ID NO:28. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:28.

The recombinant cell may have a deletion or disruption of an ER gene and include an exogenous polynucleotide that is, or may be derived from, a Jeotgalibacillus soli gene encoding the amino acid of SEQ ID NO:29. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 29.

The recombinant cell may have a deletion or disruption of an ER gene and include an exogenous polynucleotide that is, or may be derived from, a Heyndrickxia sporothermodurans gene encoding the amino acid of SEQ ID NO:30. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:30.

The recombinant cell may have a deletion or disruption of an ER gene and include an exogenous polynucleotide that is, or may be derived from, a Clostridium fungisolvens gene encoding the amino acid of SEQ ID NO:31. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:31.

The recombinant cell may have a deletion or disruption of an ER gene and include an exogenous polynucleotide that is, or may be derived from, a Neobacillus cucumis gene encoding the amino acid of SEQ ID NO:33. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 33.

The recombinant cells described herein are capable of producing xylitol, have a deletion or disruption of a native gene encoding an ER enzyme, and may include an exogenous polynucleotide sequence encoding a xylulokinase (XKS) enzyme. A recombinant cell described herein capable of producing xylitol and having a deletion or disruption of a native gene encoding an ER enzyme may additionally include an exogenous polynucleotide sequence encoding an X5PP enzyme and/or overexpress a native X5PP enzyme, an exogenous polynucleotide sequence encoding an XKS enzyme, and/or include an exogenous polynucleotide encoding a native or exogenous RPE enzyme or may have a genetic modification resulting in overexpression of a native RPE enzyme, as described herein. The exogenous polynucleotide sequence may be an exogenous xylulose sugar phosphatase (XKS) gene.

A “xylulokinase gene” and an “XKS gene” are used interchangeably herein and refer to any gene or polynucleotide that encodes a polypeptide with xylulokinase activity. As used herein, “xylulokinase activity” refer to the ability to catalyze the conversion of xylulose-5-phosphate and ADP to xylulose and ATP. The XKS gene may be derived from any suitable source. For example, the XKS gene may be derived from Saccharomyces cerevisiae. The XKS gene may encode an amino acid at least 50%, at least 60%, at least 70%, at least 80%, at least 85%, at least 90%, at least 95%, at least 97%, at least 99%, or 100% sequence identity to the amino acid sequence of at least one of SEQ ID NOs: 188 and 189. Additional description of recombinant cells capable of producing xylitol and including a polypeptide with xylulokinase activity is provided in U.S. Provisional Application No. 63/364,363, filed May 9, 2022, which is incorporated herein by reference in its entirety.

The recombinant cell may have a deletion or disruption of an ER gene and include an exogenous polynucleotide that is, or may be derived from, a Saccharomyces cerevisiae DOG1 sugar phosphatase gene encoding the amino acid of SEQ ID NO:188. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:188.

The recombinant cell may have a deletion or disruption of an ER gene and include an exogenous polynucleotide that is, or may be derived from, a Saccharomyces cerevisiae DOG2 sugar phosphatase gene encoding the amino acid of SEQ ID NO:189. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 189.

The recombinant cells described herein are capable of producing xylitol, have a deletion or disruption of a native gene encoding an ER enzyme, and an exogenous polynucleotide sequence encoding a xylitol dehydrogenase (XDH) enzyme. A recombinant cell described herein capable of producing xylitol and having a deletion or disruption of a native gene encoding an ER enzyme may additionally include an exogenous polynucleotide sequence encoding a xylitol dehydrogenase (XDH) enzyme, an exogenous polynucleotide encoding an XKS enzyme, an exogenous polynucleotide sequence encoding an X5PP enzyme and/or overexpress a native X5PP enzyme, an exogenous polynucleotide sequence encoding an XKS enzyme, and/or include an exogenous polynucleotide encoding a native or exogenous RPE enzyme or may have a genetic modification resulting in overexpression of a native RPE enzyme, as described herein. The exogenous polynucleotide sequence may be an exogenous XDH gene.

A “xylitol dehydrogenase gene” and an “XDH gene” are used interchangeably herein and refer to any gene or polynucleotide that encodes a polypeptide with xylitol dehydrogenase activity. As used herein, “xylitol dehydrogenase activity” refer to the ability to catalyze the conversion of xylulose and NADH or NADPH to xylitol and NAD⁺ or NADP⁺. The XDH gene may be derived from any suitable source. For example, the XDH gene may be derived from Pichia stipitis, Rhodobacteraceae bacterium, or Bemisia argentofolii. The XDH gene may encode an amino acid at least 50%, at least 60%, at least 70%, at least 80%, at least 85%, at least 90%, at least 95%, at least 97%, at least 99%, or 100% sequence identity to the amino acid sequence of at least one of SEQ ID NOs: 190, 191, or 192. Additional description of recombinant cells capable of producing xylitol and including a polypeptide with xylulokinase activity and a polypeptide with xylitol dehydrogenase activity is provided in U.S. Provisional Application No. 63/364,363, filed May 9, 2022, which is incorporated herein by reference in its entirety.

The recombinant cell may have a deletion or disruption of an ER gene and include an exogenous polynucleotide that is, or may be derived from, a cofactor switched Pichia stipitis XDH gene encoding the amino acid of SEQ ID NO:190. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:190.

The recombinant cell may have a deletion or disruption of an ER gene and include an exogenous polynucleotide that is, or may be derived from, a Rhodobacteraceae bacterium SDR family oxidoreductase gene encoding the amino acid of SEQ ID NO:191. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:191.

The recombinant cell may have a deletion or disruption of an ER gene and include an exogenous polynucleotide that is, or may be derived from, a Bemisia argentofolii (Silverleaf Whitefly) ketose reductase (sorbitol dehydrogenase) gene encoding the amino acid of SEQ ID NO: 192. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 192.

The exogenous polynucleotides in the recombinant cells described herein may be under the control of a promoter. For example, the exogenous nucleic acid may be operably linked to a heterologous or artificial promoter. Suitable promoters are known and described in the art. Promoters may include, but are not limited to, pyruvate decarboxylase promoter (PDC), translation elongation factor 2 promoter (TEF2), SED1, alcohol dehydrogenase 1A promoter (ADH1), hexokinase 2 promoter (HXK2), FLO5 promoter, pyruvate kinase 1 promoter (PYK1p; SEQ ID NO: 86); 6-phosphogluconate dehydrogenase promoter (6PGDp; SEQ ID NO:130); glyceraldehyde-3-phosphate dehydrogenase promoter (TDH3p; SEQ ID NO:132); translational elongation factor 1 promoter (TEFp; SEQ ID NO:133); modified TEFp (SEQ ID NO:131); phosphoglucomutase 1 promoter (PGMlp; SEQ ID NO:134); 3-phosphoglycerate kinase promoter (PGK1p; SEQ ID NO:135); enolase promoter (ENO1p; SEQ ID NO:136); asparagine synthetase promoter (ASNSp; SEQ ID NO:137); 50S ribosomal protein L1 promoter (RPLAp; SEQ ID NO: 138); and RPL16B (SEQ ID NO:139).

The exogenous nucleic acids in the recombinant cells described herein may be under the control of a terminator. For example, the exogenous nucleic acid may be operably linked to a heterologous or artificial terminator. Suitable terminators are known and described in the art. Terminators may include, but are not limited to, GAL10 terminator, PDC terminator, transaldolase terminator (TAL) 6PGD terminator (6PGDt; SEQ ID NO:140); ASNS terminator (ASNSt; SEQ ID NO: 141); ENO1 terminator (ENO1t; SEQ ID NO: 142); hexokinase 1 terminator (HXK1t; SEQ ID NO: 143); PGK1 terminator (PGK1t; SEQ ID NO:144); PGM1 terminator (PGMIt; SEQ ID NO: 145); PYK1 terminator (PYKIt; SEQ ID NO:146); RPLA terminator (RPLAt: SEQ ID NO: 147); transaldolase 1 terminator (TALIt; SEQ ID NO:148); TDH3 terminator (TDH3t; SEQ ID NO: 149); translation elongation factor 2 terminator (TEF2t; SEQ ID NO:150); triosephosphate isomerase 1 terminator (TPI1t; SEQ ID NO:151); and MpTEF1 (SEQ ID NO:289).

A promoter or terminator is “operably linked” to a given polynucleotide (e.g., a gene) if its position in the genome or expression cassette relative to said polynucleotide is such that the promoter or terminator, as the case may be, performs its transcriptional control function.

The polypeptides described herein may be provided as part of a construct. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies. The construct may be a vector including a promoter operably linked to the polynucleotide encoding a polypeptide as described herein. As used herein, the term “vector” refers to a polynucleotide capable of transporting another polynucleotide to which it has been linked. The vector may be a plasmid, which refers to a circular double-stranded DNA loop into which additional DNA segments may be integrated.

The disclosure also provides fermentation methods for the production of xylitol using the recombinant cells described herein. The fermentation methods include the step of fermenting a substrate using the genetically engineered yeasts described herein to produce xylitol. The fermentation method can include additional steps, as would be understood by a person skilled in the art. Non-limiting examples of additional process steps include maintaining the temperature of the fermentation broth within a predetermined range, adjusting the pH during fermentation, and isolating the xylitol from the fermentation broth. The fermentation process may be a fully aerobic or a partially aerobic process.

The fermentation method can be run using a suitable fermentation substrate. The substrate of the fermentation method can include glucose, sucrose, galactose, mannose, molasses, xylose, fructose, hydrolysates of starch, lignocellulosic hydrolysates, or a combination thereof. One skilled in the art will recognize what fermentation substrate is suitable for a given fermentation organism and system.

The fermentation process can be run under various conditions. The fermentation temperature, i.e., the temperature of the fermentation broth during processing, may be ambient temperature. Alternatively, or additionally, the fermentation temperature may be maintained within a predetermined range. For example, the fermentation temperature can be maintained in the range of 25 °C to 45 °C, 30 °C to 40 °C, or 32 °C to 37 °C, preferably about 35 °C. However, a skilled artisan will recognize that the fermentation temperature is not limited to any specific range or temperature recited herein and may be modified as appropriate.

The fermentation process can be run within certain oxygen uptake rate (OUR) ranges. The volumetric OUR of the fermentation process can be in the range of 5-80, 10-75, 15-70, 20-60, 30-50, or 40-50 mmol O₂/(L·h). In some embodiments, the specific OUR can be in the range of 0.05 to 10, 0.1 to 9, 0.5 to 8, 1.0 to 7, 1.5 to 6, 2 to 5, or 2.5 to 4 mmol O₂/(g cell dry weight·h). However, the volumetric or specific OURs of the fermentation process are not limited to any specific rates or ranges recited herein.

The fermentation process can be run at various cell concentrations. In some embodiments, the cell dry weight at the end of fermentation can be 5 to 40, 8 to 30, or 10 to 20 g cell dry weight/L. Further, the pitch density or pitching rate of the fermentation process can vary. In some embodiments, the pitch density can be 0.05 to 11, 0.1 to 10, or 0.25 to 8 g cell dry weight/L.

The initial dextrose concentration of the fermentation may be at least 100, 200, 250, 300, 350, or at least 400 g/L dextrose when run as a batch fermentation. The initial dextrose concentration may be between 100 to 500, 150 to 450, 400 to 200, or 250 to 350 g/L when run as a batch fermentation. When run in a fed batch fermentation process, the dextrose concentration may be at least 100, 200, 250, 300, 350, 400, or at least 450 g/L.

The fermentation process can be associated with various characteristics, such as, but not limited to, fermentation production rate, pathway fermentation yield, final titer, and peak fermentation rate. These characteristics can be affected by the selection of the yeast and/or genetic modification of the yeast used in the fermentation process. These characteristics can be affected by adjusting the fermentation process conditions. These characteristics can be adjusted via a combination of yeast selection or modification and the selection of fermentation process conditions.

The xylitol production rate of the process may be at least 0.2, 0.3, 0.5, 0.75, or at least 1.0 g L⁻¹ h⁻¹. The xylitol mass yield of the process may be at least 25, at least 30, at least 35, at least 40, at least 50, at least 55 percent, at least 65 percent, at least 70 percent, at least 75 percent, at least 80 percent, or at least 85 percent. The final xylitol titer of the process may be at least 5, 10, 20, 30, 50, 75, or 100 g/L.

The erythritol production rate of the process may be less than 0.5, 0.4, 0.3, 0.25, 0.2, 0.15, or 0.1 g L⁻¹ h⁻¹. The erythritol mass yield of the process may be less than 35%, 30%, 25%, 20%, 15%, 10%, 5%, or 1%. The final erythritol titer of the process may be less than 20, 18, 15, 12, 10, 5, 1, 0.5 g/L.

The fermentation process can be run as a dextrose-fed batch. Further, the fermentation process can be a batch process, continuous process, or semi-continuous process, as would be understood by a person skilled in the art.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

Throughout the Examples, strain numbering and sequence identification numbers are used consistently. For example, strain 1-1 in Example 4 is the same as strain 1-1 in Example 5, etc.

### Example 1—Xylitol-Phosphate Dehydrogenase Diversity

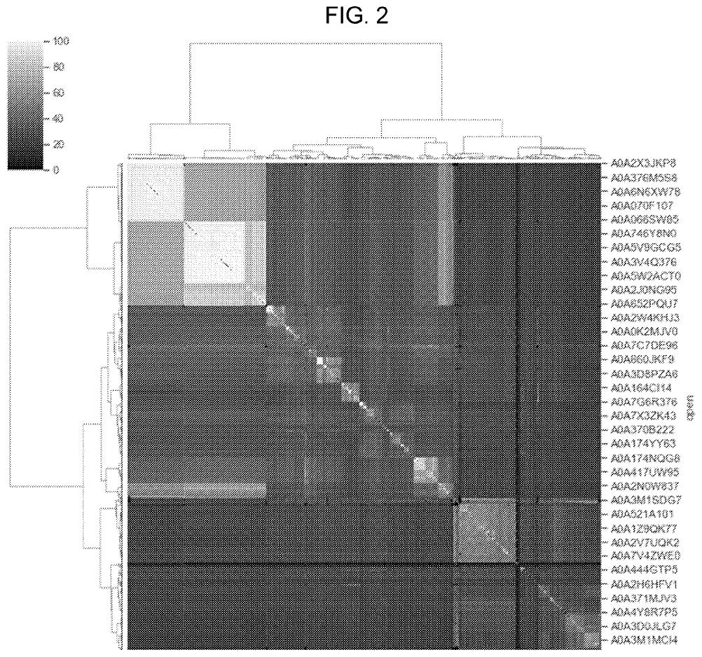

Roughly three thousand galactitol-1-phosphate-5-dehydrogenase (G1PDH)/xylitol-phosphate dehydrogenase (XPDH) enzyme sequences were obtained from Uniprot and analyzed. FIG. 2 illustrates the natural sequence diversity for this set of sequences. This set is diverse, with ~25% of the enzymes having no homologue more than 75% identical. As these enzymes tend to prefer NAD to NADP as a cofactor, the cofactor binding preferences of the homologs were assessed in a manner similar to that described by Duax et al., (“Rational proteomics I. Fingerprinting identification and cofactor specificity in the short-chain oxidoreductase (SCOR) enzyme family,” Proteins, 2003, 53 (4): 931-943). Cofactor binding pockets were identified by proximity to the Rossman fold (+23 to +30 amino acids from the GXGXXG motif (SEQ ID NO: 129)) and scored on the basis of total charge in an 8-residue window. The top 8 candidates that were predicted to use NADP were selected for further characterization, along with 4 candidates predicted to use NAD, and 3 controls.

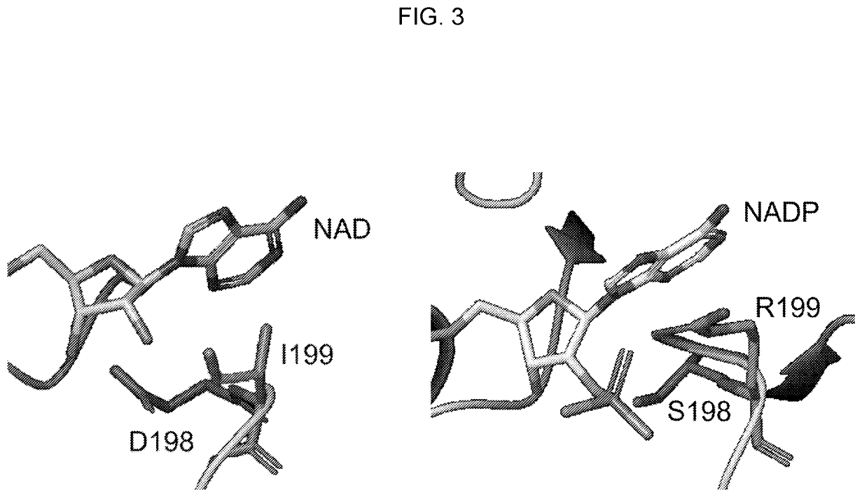

Upon further review of the structural characteristics of the predicted binding pocket for factors that may influence cofactor preference, an important aspartate residue was identified. See FIG. 3. The polypeptide of SEQ ID NO:34 and substitutions thereof were used to construct a structural homology model to predict cofactor binding pocket confirmations FIG. 3 shows the C-terminal end of the penultimate B-strand on the outside of the Rossman Fold domain. Without wishing to be bound by any particular theory, it is predicted that enzymes in which the first residue in this region (residue 198 relative to SEQ ID NO:34) is an aspartate and the second residue (residue 199 relative to SEQ ID NO:34) is a large hydrophobic amino acid (for example, isoleucine) will prefer an NAD cofactor due to the hydrogen bonding of the aspartate to the hydroxyl groups of the NAD ribose. However, enzymes in which that first residue (residue 198 relative to SEQ ID NO:34) is an alanine, glycine, or serine and the second residue (residue 199 relative to SEQ ID NO:34) is lysine or arginine will prefer an NADP cofactor as the positive charge on the lysine or arginine residue will interact with the negative charge of the phosphate of the NADP and the smaller residue in the first position allows space in the binding pocket for said phosphate. Based on this analysis, 12 additional enzymes were selected for their predicted preference for NADP. Finally, 6 additional enzymes with sequence similarity to active XPDH enzymes were selected for testing.

### Example 2—TarJ′ Diversity

Roughly eight hundred ribulose 5-phosphate reductase sequences were obtained from Uniprot and analyzed. FIG. 3 illustrates the natural sequence diversity for this set of sequences. Overall, the diversity in this set is low, as only 10% of the enzymes have no sequence similarity more than 75% identical. As these enzymes tend to prefer NADP to NAD as a cofactor, no scoring was performed, and the sequences were simply aligned in Geneious (ClustalW, default settings). Eight enzymes were selected for further analysis based on sequence similarity.

### Example 3—In Vitro Enzyme Assays

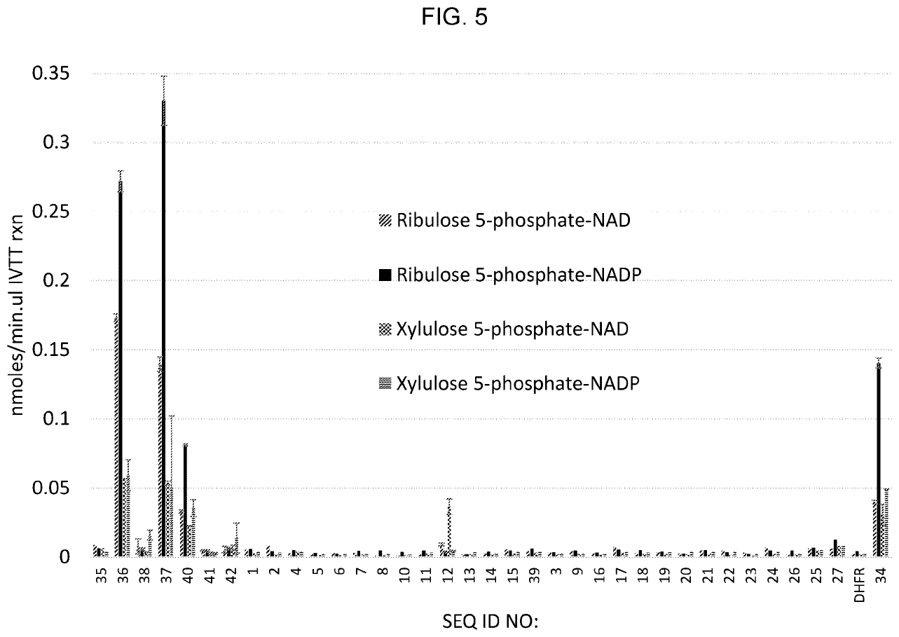

Polynucleotides encoding suspected XPDH homologs (Table 2) or TarJ′ homologs (Table 3) were cloned into a vector containing a T7 promoter and terminator for cell-free protein expression (New England Biolabs, PURExpress® In Vitro Protein Synthesis). Cell-free synthesized proteins were analyzed for activity on four substrates (ribulose 5-phosphate, xylulose 5-phosphate, ribulose, and xylulose) with either NADP or NAD cofactors. Seven enzymes (XPDH of SEQ ID NOs: 12 and 34, TarJ′ of SEQ ID NOs: 36, 37, 38, 40, and 42), were able to catalyze the reduction of either ribulose 5-phosphate or xylulose 5-phosphate (FIG. 5) but not the reduction of xylulose or ribulose (data not shown).

TABLE 2
XPDH Homologs

| | Gene Name/ | Polypeptide |
|---|---|---|
| Source Organism | Accession Numbers | SEQ ID NO: |
| Mariniphaga sediminis | D1164_16045 | 1 |
| Candidatus Dormibacteraeota bacterium | DLM67_26945 | 2 |
| Candidatus Falkowbacteria bacterium | COT93_01110 | 3 |
| Acidobacteria bacterium | DMG39_01315 | 4 |
| Spirochaetaceae bacterium | DCP56_03760 | 5 |
| Candidatus Hydrogenedentes bacterium | ENW01_11985 | 6 |
| Rhizobium sp. | E6Q76_20070 | 7 |
| Citrobacter freundii | C2U38_22630 | 8 |
| Escherichia coli | gatD | 9 |
| Escherichia coli (strain K12) | rspB | 10 |
| Lactobacillus salivarius cp400 | LSCP400_11731 | 11 |
| Clostridium difficile | WP_004454628.1 | 12 |
| Clostridium difficile | WP_011860836.1 | 13 |
| Lactobacillus rhamnosus | AAT02414.1 | 14 |
| Bacillus halodurans | WP_010896369.1 | 15 |
| Lachnospiraceae bacterium | A0A3C1WRV0_9FIRM | 16 |
| Faecalibacterium sp. OF03-6AC | DXA66_03105 | 17 |
| Agathobacter ruminis | CSX02_08830 | 18 |
| Alicyclobacillus acidoterrestris | AAC03nite_18160 | 19 |
| Ruminococcus sp. KGMB03662 | FFK04_01895 | 20 |
| Clostridiaceae bacterium | D3Z58_08610 | 21 |
| Thermoplasmata archaeon | A0A497HL05 | 22 |
| Lachnospiraceae bacterium | A0A3B918G1 | 23 |
| Prevotella marshii DSM 16973 = JCM | E0NTD1 | 24 |
| 13450 | | |
| Blautia sp. OM05-6 | A0A417S5N4 | 25 |
| Candidatus Bathyarchaeota archaeon | A0A7C4RGE8 | 26 |
| Lachnospiraceae bacterium | A0A7U9REN4 | 27 |

TABLE 3
TarJ' Homologs

| | Gene Name/ | Polypeptide |
|---|---|---|
| Source Organism | Accession Numbers | SEQ ID NO: |
| Staphylococcus aureus | AMV81374.1 | 34 |
| Staphylococcus aureus subsp. aureus 71193 | ST398NM01_0269 | 35 |
| Eubacterium ventriosum | DW918_ | 36 |
| Pradoshia sp. D12 | F7984_17520 | 37 |
| Lactobacillus plantarum EGD-AQ4 | N692_02285 | 38 |
| uncultured Ruminococcus sp. | DPQ25_03780 | 39 |
| Kandleria vitulina | SAMN04487759_1396 | 40 |
| Lactiplantibacillus plantarum | C7M34_02724 C7M35_00347 | 41 |
| | C7M36_02765 C7M47_01038 | |
| | FET70_01115 LPJSA22_01709 | |
| | Nizo2802_1951 | |
| Lachnospiraceae bacterium | IMSAGC019_01831 | 42 |

### Example 4—Genetically Modified Moniliella pollinis Strains

Strain 1-1 is the Moniliella pollinis host strain “Moniliella tomentosa var pollinis TCV364” described in U.S. Pat. No. 6,440,712, which is incorporated herein by reference in its entirety, and deposited under the Budapest Treaty at BCCM/MUCL (Belgian Coordinated Collections of Micro-organisms/Mycothèque de l'Université Catholique de Louvain by Eridania Béghin Say, Vilvoorde R&D Centre, Havenstraat 84, B-1800 Vilvoorde) on Mar. 28, 1997 under number MUCL40385. Table 4 below lists various Moniliella pollinis strains, including information on the parent strain, the sequence with which the parent strain was transformed, and characterizations of the expression cassette(s) contained on the transformed sequence. Each “XPDH/TarJ′ Homolog Expression Cassette” contained, in order, a 5′ ER1 flanking sequence (SEQ ID NO:85), an MpPYK1 promoter (SEQ ID NO:86), a gene encoding the indicated XPDH or TarJ′ homolog (one of SEQ ID NOs: 87-128), an Mp6PGD terminator (SEQ ID NO: 140), and a 5′ portion of a G418 resistance gene expression cassette (SEQ ID NO:175). Each “Selectable Marker Cassette” contained, in order, a 3′ portion of a G418 resistance gene expression cassette (SEQ ID NO:172), an MpTEF2 terminator (SEQ ID NO:150), and a 3′ ER1 flanking sequence (SEQ ID NO:160). Upon bipartite transformation with both the XPDH/TarJ′ Homolog Expression Cassette and the Selectable Marker Cassette, the two cassettes recombine for integration of both the nucleotide sequence encoding the XPDH or TarJ′ homolog and the G418 resistance marker at the ER1 locus.

The indicated Moniliella pollinis parent strain was transformed with the indicated sequence(s) by first protoplasting the parent strain by adding an enzyme mixture containing 0.6M MgSO₄, 7.5 g/L driselase, and 12.5 g/L Trichoderma harzianum lysing enzyme to a mycelial pellet of the parent strain. Protoplasts were then pelleted, washed with 0.6M MgSO₄, and resuspended in STC medium (0.6M sucrose, 50 mM CaCl2, 10 mM Tris-HCl, pH 7.5). Bipartite transformations were prepared by adding 100 μg single stranded salmon sperm DNA and 1.5 to 5 μg each of the 5′ and 3′ DNA transformation fragments (3-10 μg total; see Table 4 for list of fragments) to approximately 200 μL protoplast mixture (10⁸ cells/mL). 1 mL 50% PEG in STC medium was then added to the salmon sperm DNA, transformation DNA, and protoplast mixture and the resulting combination was incubated for 15 minutes at room temperature. Following incubation, recovery broth (0.4M sucrose, 1 g/L yeast extract, 1 g/L malt extract, 10 g/L glucose, pH 4.5) was added to the mixture and incubated at 27 °C, 100 rpm, for 16 to 24 hours. Following the incubation, protoplasts were pelleted by centrifugation and resuspended in 1 mL PBS.

The resuspended protoplasts were plated on PDA+250 mg/L geneticin (G418) selection plates and incubated at 30-35 °C for at least 2-4 days until transformants grow. Resulting transformants were evaluated by colony PCR for integration of the indicated sequence. A PCR verified isolate was then designated as the indicated strain number. In some instances, more than one PCR verified isolate, e.g., “sister” isolates, are indicated by letters following the strain number. For example, strain 1-2 has 5 sister isolates, strains 1-2a, 1-2b, 1-2c, 1-2d, and 1-2e.

For example, Strain 1-1 was transformed with SEQ ID NO:43 and SEQ ID NO:44. SEQ ID NO: 43 contains (i) 3′ flanking DNA for targeted chromosomal integration into the ER1 locus (SEQ ID NO:162), and (ii) a 3′ portion of the G418 resistance gene selectable marker (SEQ ID NO: 172). SEQ ID NO:44 contains (i) an expression cassette for the XPDH homolog from M. sediminis, SEQ ID NO:87 encoding the amino acid sequence of SEQ ID NO:1, under the control of the PYK1 promoter of SEQ ID NO:86 and the PGD terminator of SEQ ID NO:140; (ii) 5′ flanking DNA for targeted chromosomal integration into the ER1 locus (SEQ ID NO:85); and (iii) a 5′ portion of the G418 resistance gene selectable marker (SEQ ID NO:175). Transformants were selected on PDA+250 mg/L geneticin (G418) selection plates and incubated at 30-35 °C for at least 2 days until transformants grow. Resulting transformants were streaked for single colony isolation on PDA+geneticin (G418) plates and single colonies were selected. Selected colonies were evaluated by colony PCR for integration of the indicated sequence. PCR verified isolates were designated strains 1-2a, 1-2b, 1-2c, 1-2d, and 1-2e.

TABLE 4

| | | | | | Selectable |
|---|---|---|---|---|---|
| | | XPDH/TarJ' Homolog Expression Cassette | | | Marker |
| | | | | Encoded | Cassette |
| | Parent | Transformation | Nucleotide | Polypeptide | Transformation |
| Strain | Strain | SEQ ID NO: | SEQ ID NO: | SEQ ID NO: | SEQ ID NO: |
| 1-2a-e | 1-1 | 44 | 87 | 1 | 43 |
| 1-3a-e | 1-1 | 45 | 88 | 2 | 43 |
| 1-4a-e | 1-1 | 46 | 89 | 3 | 43 |
| 1-5a-e | 1-1 | 47 | 90 | 4 | 43 |
| 1-6a-e | 1-1 | 48 | 91 | 5 | 43 |
| 1-7a-e | 1-1 | 49 | 92 | 6 | 43 |
| 1-8a-d | 1-1 | 50 | 93 | 7 | 43 |
| 1-9a-e | 1-1 | 51 | 94 | 8 | 43 |
| 1-10a-e | 1-1 | 52 | 95 | 9 | 43 |
| 1-11a-e | 1-1 | 53 | 96 | 10 | 43 |
| 1-12a-e | 1-1 | 54 | 97 | 11 | 43 |
| 1-13a-f | 1-1 | 55 | 98 | 12 | 43 |
| 1-14a-e | 1-1 | 56 | 99 | 13 | 43 |
| 1-15a-f | 1-1 | 57 | 100 | 14 | 43 |
| 1-16a-e | 1-1 | 58 | 101 | 15 | 43 |
| 1-17a-e | 1-1 | 59 | 102 | 16 | 43 |
| 1-18a-e | 1-1 | 60 | 103 | 17 | 43 |
| 1-19a-e | 1-1 | 61 | 104 | 18 | 43 |
| 1-20a-e | 1-1 | 62 | 105 | 19 | 43 |
| 1-21a-e | 1-1 | 63 | 106 | 20 | 43 |
| 1-22a-e | 1-1 | 64 | 107 | 21 | 43 |
| 1-23a-e | 1-1 | 65 | 108 | 22 | 43 |
| 1-24a-e | 1-1 | 66 | 109 | 23 | 43 |
| 1-25a-e | 1-1 | 67 | 110 | 24 | 43 |
| 1-26a-e | 1-1 | 68 | 111 | 25 | 43 |
| 1-27a-e | 1-1 | 69 | 112 | 26 | 43 |
| 1-28a-e | 1-1 | 70 | 113 | 27 | 43 |
| 1-29a-e | 1-1 | 71 | 114 | 28 (Alkalihalobacillus | 43 |
| | | | | ligniniphilus XPDH | |
| | | | | WP_017729640.1) | |
| 1-30a-e | 1-1 | 72 | 115 | 29 (Jeotgalibacillus | 43 |
| | | | | soli XPDH | |
| | | | | WP_041086875.1) | |
| 1-31a-e | 1-1 | 73 | 116 | 30 (Heyndrickxia | 43 |
| | | | | sporothermodurans | |
| | | | | XPDH | |
| | | | | WP_066235909.1) | |
| 1-32a-e | 1-1 | 74 | 117 | 31 (Clostridium | 43 |
| | | | | fungisolvens XPDH | |
| | | | | WP_183276691.1) | |
| 1-33a-e | 1-1 | 75 | 118 | 32 (Alkalihalobacillus | 43 |
| | | | | nanhaiisediminis | |
| | | | | XPDH | |
| | | | | WP144449381.1) | |
| 1-34a-e | 1-1 | 76 | 119 | 33 (Neobacillus | 43 |
| | | | | cucumis XHPD | |
| | | | | WP_198313751.1) | |
| 1-35a-d | 1-1 | 77 | 120 | 34 | 43 |
| 1-36a-e | 1-1 | 78 | 121 | 35 | 43 |
| 1-37a-d | 1-1 | 79 | 122 | 36 | 43 |
| 1-38a-f | 1-1 | 80 | 123 | 37 | 43 |
| 1-39a-f | 1-1 | 81 | 124 | 38 | 43 |
| 1-40a-e | 1-1 | 82 | 125 | 39 | 43 |
| 1-41a-e | 1-1 | 83 | 127 | 41 | 43 |
| 1-42a-f | 1-1 | 84 | 128 | 42 | 43 |

### Example 5-Shake Flask Fermentation Assay

Strains 1-1, 1-35a-d, 1-37a-d, 1-38a-f, 1-39a-f, 1-42a-f, 1-13a-f, and 1-15a-f (outlined in Table 4 above), were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

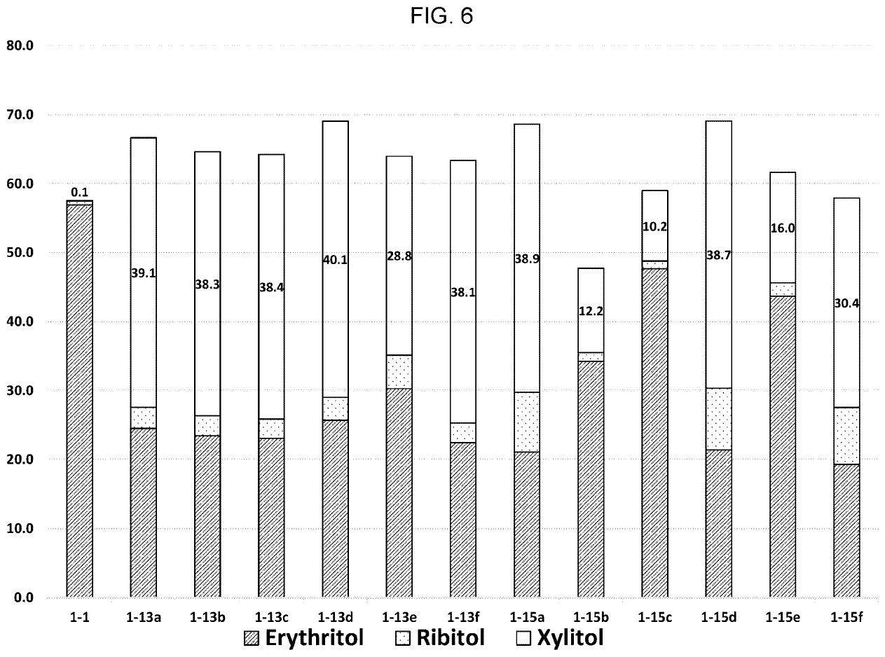

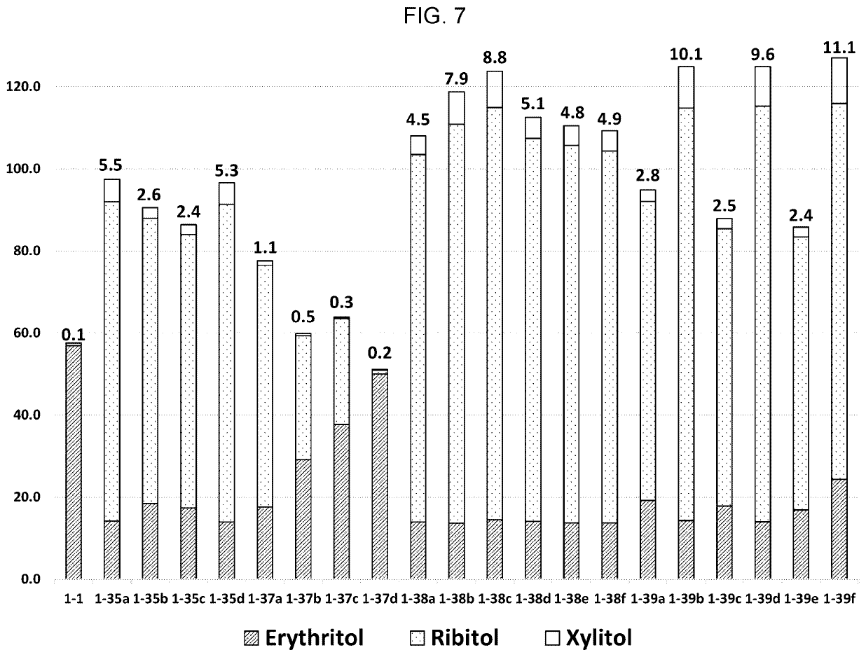

A 250 ml non-baffled flask containing production medium (Table 5) was inoculated with 0.8 mL of the seed culture to form the production culture. The production culture was incubated at 35 °C and 250 rpm. Samples were taken from the production culture after 72 and 96 hours of incubation. Samples were analyzed for glucose, ribitol, xylitol, erythritol, glycerol, and ethanol by high performance liquid chromatography with refractive index detector. Fermentation results are reported in Table 6 and FIGS. 6 and 7.

TABLE 5
Production Medium

| Component | Concentration (units) |
|---|---|
| Glucose | 300 (g/L) |
| KH2PO4 | 1.27 (g/L) |
| (NH4)2HPO4 | 0.13 (g/L) |
| (NH4)2SO4 | 1.80 (g/L) |
| Urea | 2.85 (g/L) |
| Citric Acid | 200.0 (mg/L) |
| MgSO4—7H2O | 690.2 (mg/L) |
| FeSO4—7H2O | 32.35 (mg/L) |
| ZnSO4—7H2O | 4.40 (mg/L) |
| MnSO—4H2O | 0.53 (mg/L) |
| CuSO4—5H2O | 0.45 (mg/L) |
| CaCl2—2H2O | 91.60 (mg/L) |
| Na2SO4 | 154.50 (mg/L) |
| Thiamine-HCl | 10 (mg/L) |
| Choline-Cl | 100 (mg/L) |
| Antifoam CF-32 | 1.00 (g/L) |

While PCR verification indicated that the transformed polynucleotide sequence was present in the indicated strains, further analysis indicated that in some strains, the sequence was not correctly integrated at the ER1 locus. Further analysis indicated that strains 1-35a, 1-37a-d, 1-38a-c, 1-39d-f, 1-42a-b, 1-42d, 1-13a-b, 1-13d-e, 1-15b-c, and 1-15e-f include the transformed polynucleotide sequence, but it is not integrated at the ER1 locus.

TABLE 6
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-1 | 54.2270 | 0.5672 | 0.1156 | 56.9108 | 14.4101 | 45.2717 |
| 1-35a | 19.0690 | 77.8018 | 5.5078 | 14.1262 | 11.6922 | 45.6143 |
| 1-35b | 22.4343 | 69.5363 | 2.6051 | 18.4159 | 6.7667 | 51.1619 |
| 1-35c | 31.4030 | 66.5826 | 2.4193 | 17.3705 | 7.5255 | 49.2545 |
| 1-35d | 28.2827 | 77.4169 | 5.2674 | 13.9265 | 7.1954 | 49.1733 |
| 1-37a | 42.3156 | 58.8415 | 1.1308 | 17.6218 | 8.1394 | 44.6216 |
| 1-37b | 92.0525 | 30.1794 | 0.5249 | 29.1345 | 17.4800 | 28.4251 |
| 1-37c | 77.9520 | 25.8442 | 0.3365 | 37.6779 | 28.5369 | 33.4608 |
| 1-37d | 62.2726 | 0.9251 | 0.2062 | 50.0038 | 18.7245 | 45.7655 |
| 1-38a | 19.5123 | 89.5643 | 4.5079 | 13.9258 | 6.0135 | 46.2436 |
| 1-38b | 15.4952 | 97.2570 | 7.8962 | 13.6124 | 6.7302 | 35.1221 |
| 1-38c | 9.1530 | 100.4624 | 8.8382 | 14.4767 | 6.4634 | 41.8789 |
| 1-38d | 3.5262 | 93.3559 | 5.1135 | 14.0524 | 7.8424 | 46.9099 |
| 1-38e | 5.8345 | 91.9564 | 4.7706 | 13.7265 | 7.3560 | 48.8018 |
| 1-38f | 5.6278 | 90.6644 | 4.9442 | 13.6917 | 7.7348 | 47.5447 |
| 1-39a | 18.6244 | 72.8649 | 2.8000 | 19.1466 | 8.1946 | 40.2287 |
| 1-39b | 2.3933 | 100.5639 | 10.0874 | 14.2583 | 6.9797 | 44.4668 |
| 1-39c | 31.7163 | 67.5513 | 2.4932 | 17.8548 | 7.1724 | 47.5427 |
| 1-39d | 3.3290 | 101.3044 | 9.6496 | 13.9511 | 6.9558 | 46.4375 |
| 1-39e | 38.9049 | 66.5485 | 2.4033 | 16.8345 | 7.1407 | 48.7270 |
| 1-39f | 0.8569 | 91.5653 | 11.1004 | 24.3341 | 6.1712 | 37.6166 |
| 1-42a | 53.2303 | 1.3617 | 0.1586 | 62.1601 | 16.1305 | 44.7835 |
| 1-42b | 70.3640 | 0.5390 | 0.1319 | 52.0362 | 13.1695 | 44.2371 |
| 1-42c | 54.3271 | 0.6740 | 0.1317 | 61.0264 | 17.1397 | 44.9797 |
| 1-42d | 55.3052 | 0.7063 | 0.1484 | 63.2909 | 12.3513 | 43.1762 |
| 1-42e | 61.0003 | 0.5738 | 0.1502 | 56.8237 | 13.3096 | 39.6208 |
| 1-42f | 64.0496 | 0.5451 | 0.1352 | 55.2579 | 13.6874 | 42.7263 |
| 1-13a | 58.2322 | 3.0713 | 39.0606 | 24.5223 | 9.9512 | 43.5850 |
| 1-13b | 64.5326 | 2.8987 | 38.2994 | 23.4455 | 9.2364 | 42.1528 |
| 1-13c | 62.8811 | 2.8168 | 38.3638 | 23.0771 | 9.2614 | 43.8806 |
| 1-13d | 47.8236 | 3.3178 | 40.0543 | 25.7124 | 9.9847 | 40.8596 |
| 1-13e | 59.3667 | 4.8671 | 28.8414 | 30.2839 | 9.5968 | 42.2960 |
| 1-13f | 66.1336 | 2.8202 | 38.0642 | 22.4811 | 8.8765 | 42.3954 |
| 1-15a | 58.6182 | 8.6323 | 38.8703 | 21.1152 | 9.3006 | 43.9871 |
| 1-15b | 78.7837 | 1.2617 | 12.2069 | 34.2608 | 11.5806 | 43.9409 |
| 1-15c | 55.9848 | 1.1212 | 10.2043 | 47.6605 | 12.0438 | 44.2130 |
| 1-15d | 54.0847 | 8.9671 | 38.7047 | 21.4173 | 9.3022 | 44.1645 |
| 1-15e | 60.7735 | 1.9279 | 16.0149 | 43.7036 | 11.1713 | 46.0607 |
| 1-15f | 102.6556 | 8.2515 | 30.3843 | 19.3150 | 13.0177 | 32.7172 |

### Example 6—Shake Flask Fermentation Assay

Strains 1-13c, 1-29a-e, 1-33a-e, and 1-34a-e (outlined in Table 4 above), were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

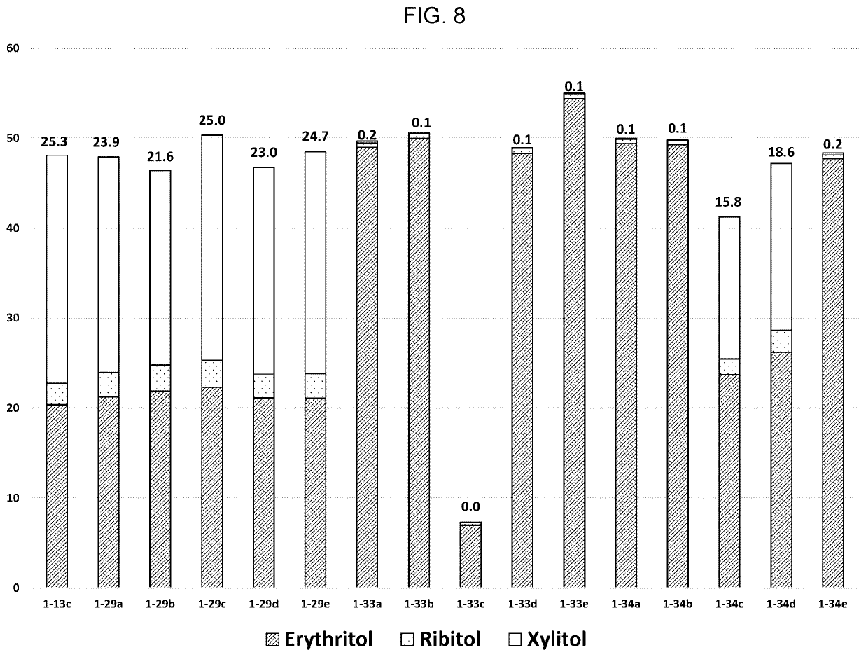

A 250 ml non-baffled flask containing production medium (Table 5) was inoculated with 0.8 mL of the seed culture to form the production culture. The production culture was incubated at 35 °C and 250 rpm. Samples were taken from the production culture after 96 hours of incubation. Samples were analyzed for glucose, ribitol, xylitol, erythritol, glycerol, and ethanol by high performance liquid chromatography with refractive index detector. Fermentation results are reported in Table 7 and FIG. 8.

As seen in FIG. 8, while sister strains 1-34c and 1-34d produced 15.8 and 18.6 g/L xylitol, respectively, strains 1-34a, 1-34b, and 1-34e did not produce significantly more xylitol than wild-type (strain 1-1, FIG. 6). While strains 1-34a, 1-34b, and 1-34e were initially PCR verified, it was later determined that the integrated polynucleotide, which should encode the N. cucumis XPDH homolog, contained a frameshift mutation and no functional XPDH was expressed. Therefore, while the results appear varied, they are in fact consistent given that strains 1-34a, 1-34b, and 1-34e did not contain a polynucleotide that encoded a functional XPDH.

TABLE 7
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-13c | 104.7609 | 2.4041 | 25.3431 | 20.3872 | 8.3483 | 28.8597 |
| 1-29a | 107.59 | 2.7375 | 23.9236 | 21.2592 | 8.327 | 29.7606 |
| 1-29b | 117.0722 | 2.8833 | 21.624 | 21.9028 | 7.5941 | 30.1983 |
| 1-29c | 92.8027 | 3.0122 | 25.0203 | 22.3197 | 8.607 | 34.1865 |
| 1-29d | 113.7418 | 2.654 | 22.973 | 21.1195 | 7.992 | 30.2967 |
| 1-29e | 106.1983 | 2.7505 | 24.6828 | 21.1105 | 8.3573 | 30.0138 |
| 1-33a | 102.5508 | 0.4847 | 0.2175 | 48.9982 | 9.7203 | 31.4812 |
| 1-33b | 101.4479 | 0.507 | 0.0751 | 50.0129 | 9.8179 | 31.3012 |
| 1-33c | 231.4215 | 0.2896 | 0.0353 | 6.9913 | 16.2002 | 6.2899 |
| 1-33d | 107.2532 | 0.5626 | 0.0542 | 48.3365 | 10.36 | 31.982 |
| 1-33e | 89.76 | 0.5258 | 0.0847 | 54.4229 | 10.6124 | 27.6685 |
| 1-34a | 97.4677 | 0.487 | 0.0849 | 49.4197 | 10.4031 | 31.8369 |
| 1-34b | 99.6607 | 0.4724 | 0.0682 | 49.2765 | 9.7575 | 31.2798 |
| 1-34c | 125.3303 | 1.7402 | 15.7865 | 23.7361 | 8.5658 | 28.3016 |
| 1-34d | 109.6517 | 2.4549 | 18.5615 | 26.2014 | 8.1791 | 30.7102 |
| 1-34e | 92.4856 | 0.434 | 0.22 | 47.7303 | 9.2374 | 32.5853 |

### Example 7—Shake Flask Fermentation Assay

Strains 1-13c, 1-17a-e, 1-18a-e, 19a-e, 1-21a-e, 1-22a-e, 1-23a-e, 1-24a-e, 1-25a-e, and 1-27a-d (outlined in Table 4 above), were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

A 250 ml non-baffled flask containing production medium (Table 5) was inoculated with 0.8 mL of the seed culture to form the production culture. The production culture was incubated at 35 °C and 250 rpm. Samples were taken from the production culture after 96 hours of incubation. Samples were analyzed for glucose, ribitol, xylitol, erythritol, glycerol, and ethanol by high performance liquid chromatography with refractive index detector. Fermentation results are reported in Table 8.

TABLE 8
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-13a | 98.6422 | 2.6387 | 24.7712 | 25.2307 | 13.8005 | 28.8903 |
| 1-17a | 74.915 | 0.5991 | 0.2543 | 60.8569 | 13.0048 | 33.3547 |
| 1-17b | 79.2438 | 0.577 | 0.0725 | 58.9302 | 12.8216 | 35.3409 |
| 1-17c | 151.0953 | 0.4237 | 0.0975 | 24.7655 | 21.0532 | 20.2202 |
| 1-17d | 90.0013 | 1.4266 | 0.0645 | 55.808 | 18.8406 | 27.8706 |
| 1-17e | 84.2381 | 0.7091 | 0.0428 | 59.7786 | 15.6082 | 29.9387 |
| 1-18a | 187.0194 | 0.3163 | 0.1069 | 21.6446 | 15.8366 | 10.9522 |
| 1-18b | 83.2939 | 0.6768 | 0.0524 | 63.3983 | 14.7387 | 31.6461 |
| 1-18c | 88.21 | 0.6902 | 0.0625 | 59.74 | 14.7915 | 31.5115 |
| 1-18d | 76.4267 | 0.8183 | 0.0692 | 57.1505 | 16.2282 | 34.0999 |
| 1-18e | 89.0621 | 0.7336 | 0.0841 | 42.5495 | 26.7038 | 25.7627 |
| 1-19a | 94.518 | 0.6166 | 0.0801 | 54.4468 | 18.8522 | 28.9871 |
| 1-19b | 83.559 | 0.6632 | 0.0659 | 60.2352 | 14.9726 | 31.2065 |
| 1-19c | 93.3168 | 0.7412 | 0.0646 | 60.6521 | 14.3042 | 29.9464 |
| 1-19d | 165.946 | 0.4311 | 0.1236 | 24.1998 | 15.9581 | 19.0694 |
| 1-19e | 78.3357 | 0.675 | 0.0637 | 61.3653 | 15.847 | 29.361 |
| 1-21a | 77.8724 | 0.882 | 0.0789 | 65.2592 | 16.0186 | 31.302 |
| 1-21b | 85.2124 | 0.6777 | 0.0619 | 56.1455 | 15.4518 | 33.3606 |
| 1-21c | 85.008 | 1.0769 | 0.0802 | 58.6289 | 14.8934 | 32.8785 |
| 1-21d | 83.4408 | 1.9855 | 0.0373 | 56.0976 | 14.2115 | 34.3856 |
| 1-21e | 60.5121 | 2.039 | 0.0418 | 63.3641 | 15.5567 | 31.3166 |
| 1-22a | 96.2241 | 0.8385 | 0.0644 | 62.4003 | 13.1106 | 29.8987 |
| 1-22b | 90.5783 | 0.5506 | 0.0722 | 54.306 | 13.9584 | 34.1293 |
| 1-22c | 85.3285 | 0.6442 | 0.0595 | 60.2244 | 15.0443 | 31.2101 |
| 1-22d | 87.0015 | 0.6859 | 0.0824 | 59.9578 | 14.9889 | 31.3812 |
| 1-22e | 78.5983 | 0.5432 | 0.0718 | 58.3192 | 14.9655 | 30.6604 |
| 1-23a | 84.0794 | 0.4999 | 0.109 | 52.3855 | 13.872 | 35.266 |
| 1-23b | 84.7042 | 0.5799 | 0.0696 | 58.9566 | 15.0869 | 33.9501 |
| 1-23c | 87.9261 | 0.7983 | 0.0538 | 55.5429 | 17.3164 | 30.1523 |
| 1-23d | 87.2081 | 0.6745 | 0.0623 | 59.4328 | 14.8259 | 31.3395 |
| 1-23e | 82.9585 | 1.0568 | 0.0795 | 62.242 | 18.8816 | 23.5949 |
| 1-24a | 86.7546 | 0.534 | 0.0725 | 53.0638 | 13.0757 | 34.4192 |
| 1-24b | 104.4802 | 0.583 | 0.0821 | 48.7903 | 14.9732 | 29.8021 |
| 1-24c | 104.5435 | 0.563 | 0.0872 | 48.1204 | 14.5086 | 31.0036 |
| 1-24d | 169.2148 | 0.425 | 0.1078 | 25.2327 | 14.0784 | 19.6567 |
| 1-24e | 78.1939 | 0.5172 | 0.082 | 55.4618 | 15.2743 | 36.423 |
| 1-25a | 85.5913 | 0.8201 | 0.0711 | 57.2974 | 18.9442 | 29.1298 |
| 1-25b | 83.3265 | 0.6891 | 0.0801 | 60.4083 | 15.034 | 31.4877 |
| 1-25c | 139.0213 | 0.6194 | 0.1079 | 28.2606 | 22.9621 | 22.0197 |
| 1-25d | 137.1956 | 0.6387 | 0.1254 | 28.3686 | 22.7144 | 22.2391 |
| 1-25e | 78.0893 | 0.6945 | 0.0792 | 61.3866 | 15.5101 | 31.7923 |
| 1-27a | 54.0049 | 0.8002 | 0.0653 | 73.3789 | 15.1766 | 36.3078 |
| 1-27b | 88.2258 | 0.5067 | 0.0543 | 53.4474 | 13.5761 | 34.0372 |
| 1-27c | 114.4683 | 0.7334 | 0.0729 | 43.7274 | 17.9455 | 26.3899 |
| 1-27d | 76.682 | 0.5437 | 0.0552 | 58.8882 | 12.8684 | 38.8414 |

### Example 8—Shake Flask Fermentation Assay

Strains 1-13c, 1-3a-e, 1-10a-e, 1-11a-e, 1-12a-e, 1-14a-e, 1-16a-e, 1-28a-e, and 1-2a-e (outlined in Table 4 above), were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

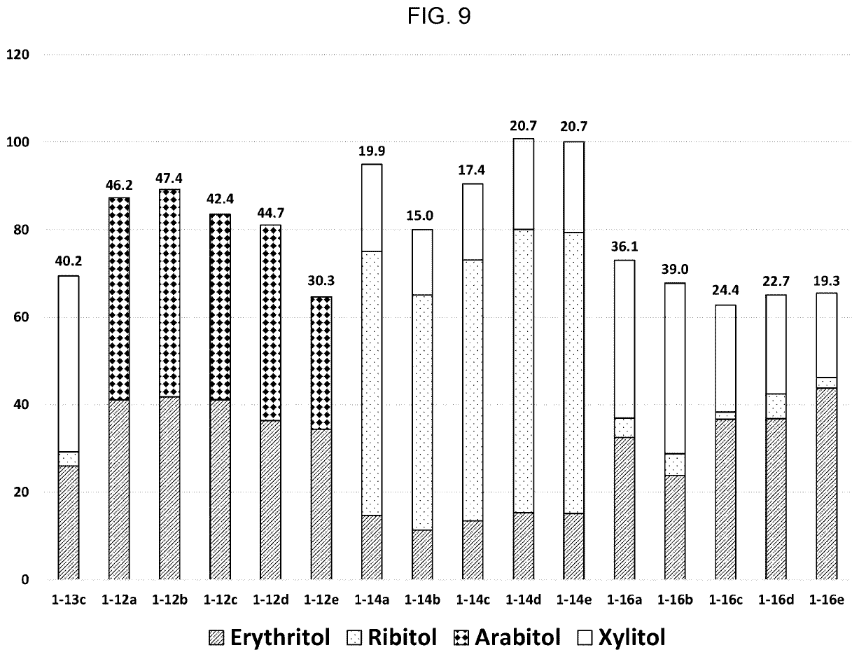

A 250 ml non-baffled flask containing production medium (Table 5) was inoculated with 0.8 mL of the seed culture to form the production culture. The production culture was incubated at 35 °C and 250 rpm. Samples were taken from the production culture after 72 and 96 hours of incubation. Samples were analyzed for glucose, ribitol, xylitol, erythritol, glycerol, and ethanol by high performance liquid chromatography with refractive index detector. Fermentation results are reported in Table 9 and FIG. 9.

While PCR verification indicated that the transformed polynucleotide sequence was present in the indicated strains, further analysis indicated that in some strains, the sequence was not correctly integrated at the ER1 locus. Further analysis indicated that strain 1-16b-e includes the transformed polynucleotide sequence, but it is not at the ER1 locus. Further analysis was inconclusive on the integration location in strains 1-2c and 1-2d.

TABLE 9
96-hour Shake Flask Results

| | Fermentation Broth Analyte (g/L) | | | | | | |
|---|---|---|---|---|---|---|---|
| Strain | Glucose | Ribitol | Xylitol | Erythritol | Glycerol | Ethanol | Arabitol |
| 1-13c | 52.365 | 3.1859 | 40.2435 | 26.0209 | 11.1145 | 37.6398 | |
| 1-3a | 58.7394 | 0.6129 | 0.3235 | 59.7362 | 15.12 | 40.891 | |
| 1-3b | 40.836 | 0.4677 | 0.1051 | 49.0415 | 15.6583 | 51.2491 | |
| 1-3c | 55.8652 | 0.5887 | 0.1253 | 59.2426 | 14.1511 | 42.761 | |
| 1-3d | 47.4122 | 0.5038 | 0.1279 | 50.8589 | 16.8177 | 41.1038 | |
| 1-3e | 45.8606 | 0.5127 | 0.1267 | 50.1914 | 16.3743 | 48.2382 | |
| 1-10a | 58.5369 | 0.5891 | 0.1201 | 57.9712 | 15.3885 | 40.4663 | |
| 1-10b | 58.9318 | 0.6075 | 0.1492 | 57.9368 | 14.8489 | 41.9797 | |
| 1-10c | 67.992 | 0.4588 | 0.142 | 49.0939 | 14.307 | 40.394 | |
| 1-10d | 46.0003 | 0.6906 | 0.0995 | 62.4692 | 19.849 | 32.9163 | |
| 1-10e | 55.0568 | 0.655 | 0.136 | 61.0313 | 15.9185 | 38.2694 | |
| 1-11a | 51.9376 | 0.429 | 0.0526 | 60.4417 | 14.8425 | 40.9479 | |
| 1-11b | 45.6503 | 0.554 | 0.1326 | 61.3534 | 18.1726 | 44.3344 | |
| 1-11c | 5.6815 | 1.5471 | 0.0801 | 86.3607 | 27.719 | 41.9823 | |
| 1-11d | 40.3772 | 0.5489 | 0.1304 | 61.8324 | 18.171 | 41.9493 | |
| 1-11e | 53.0558 | 0.6124 | 0.115 | 60.6504 | 15.489 | 39.7766 | |
| 1-12a | 30.456 | n.a. | n.a. | 41.1173 | 9.6187 | 44.7618 | 46.2065 |
| 1-12b | 28.2127 | n.a. | n.a. | 41.7725 | 9.3555 | 45.1461 | 47.4416 |
| 1-12c | 45.2228 | n.a. | n.a. | 41.1422 | 9.0341 | 39.2179 | 42.4228 |
| 1-12d | 30.6334 | n.a. | n.a. | 36.381 | 10.0025 | 39.6166 | 44.7009 |
| 1-12e | 81.9896 | n.a. | n.a. | 34.348 | 12.8173 | 30.9024 | 30.2901 |
| 1-14a | 36.1578 | 60.3639 | 19.8883 | 14.677 | 6.9789 | 39.9443 | |
| 1-14b | 29.7316 | 53.6709 | 14.9948 | 11.3645 | 12.08 | 43.9787 | |
| 1-14c | 27.9273 | 59.5839 | 17.4099 | 13.471 | 7.8201 | 45.0742 | |
| 1-14d | 13.2601 | 64.6881 | 20.7494 | 15.3596 | 7.6729 | 41.5184 | |
| 1-14e | 24.8568 | 64.1917 | 20.7387 | 15.176 | 7.2937 | 39.5752 | |
| 1-16a | 53.8198 | 4.4367 | 36.058 | 32.5055 | 10.8652 | 39.1211 | |
| 1-16b | 52.0735 | 4.9579 | 38.9526 | 23.8416 | 10.1467 | 41.0993 | |
| 1-16c | 55.1727 | 1.6863 | 24.425 | 36.6549 | 11.7111 | 41.8186 | |
| 1-16d | 45.5429 | 5.6345 | 22.6578 | 36.7881 | 11.2977 | 41.0565 | |
| 1-16e | 41.996 | 2.3924 | 19.2866 | 43.8223 | 17.3556 | 40.5198 | |
| 1-28a | 57.3853 | 0.6449 | 0.2041 | 58.1115 | 15.5452 | 41.2408 | |
| 1-28b | 58.7477 | 0.5753 | 0.1016 | 56.9917 | 14.4135 | 41.1243 | |
| 1-28c | 59.1807 | 0.5957 | 0.1202 | 57.6647 | 14.5907 | 42.3829 | |
| 1-28d | 32.0943 | 0.6578 | 0.1353 | 62.5783 | 18.1666 | 40.946 | |
| 1-28e | 56.5966 | 0.5859 | 0.1193 | 57.5414 | 14.0459 | 41.5865 | |
| 1-2a | 50.0384 | 0.7556 | 0.1162 | 61.3047 | 15.3111 | 41.1167 | |
| 1-2b | 34.3286 | 0.79 | 0.1848 | 58.4062 | 19.6802 | 48.8752 | |
| 1-2c | 43.167 | 0.7455 | 0.0948 | 70.8234 | 11.4465 | 42.3114 | |
| 1-2d | 41.842 | 0.6423 | 0.1082 | 63.7359 | 15.0666 | 39.6665 | |
| 1-2e | 47.5807 | 0.6122 | 0.1415 | 60.543 | 13.8641 | 43.2135 | |

### Example 9-Shake Flask Fermentation Assay

Strains 1-13c, 1-8a-d, 1-26a-e, 1-36a-e, 1-41a-e, 1-40a-e, and 1-20a-e (outlined in Table 4 above), were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

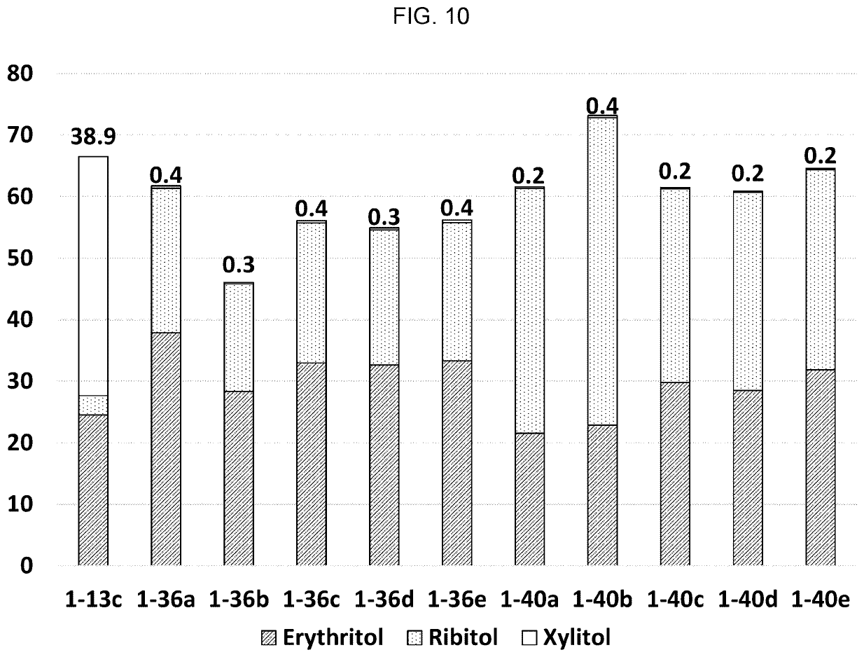

A 250 ml non-baffled flask containing production medium (Table 5) was inoculated with 0.8 mL of the seed culture to form the production culture. The production culture was incubated at 35 °C and 250 rpm. Samples were taken from the production culture after 96 hours of incubation. Samples were analyzed for glucose, ribitol, xylitol, erythritol, glycerol, and ethanol by high performance liquid chromatography with refractive index detector. Fermentation results are reported in Table 10 and FIG. 10.

While PCR verification indicated that the transformed polynucleotide sequence was present in the indicated strains, further analysis indicated that in some strains, the sequence was not correctly integrated at the ER1 locus. Further analysis indicated that strains 1-8c, 1-8d and 1-41c include the transformed polynucleotide sequence, but it is not integrated at the ER1 locus. Further analysis was inconclusive on integration locus in strains 1-36a, 1-41b, 1-41e, and 1-20a-e.

TABLE 10
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-13c | 43.7126 | 3.1176 | 38.8613 | 24.5197 | 9.666 | 42.859 |
| 1-8a | 46.3518 | 0.537 | 0.3381 | 57.0411 | 14.4597 | 46.2915 |
| 1-8b | 47.5195 | 0.5334 | 0.1431 | 56.9461 | 13.7069 | 47.0977 |
| 1-8c | 48.0894 | 0.5594 | 0.1063 | 55.9984 | 13.6091 | 47.8657 |
| 1-8d | 37.4186 | 0.6121 | 0.1105 | 56.5403 | 13.8863 | 51.6109 |
| 1-26a | 44.7559 | 0.6392 | 0.1211 | 58.7843 | 13.0302 | 45.1907 |
| 1-26b | 26.8789 | 0.7454 | 0.1365 | 65.6702 | 14.7237 | 48.8557 |
| 1-26c | 42.4506 | 0.7303 | 0.1018 | 65.269 | 12.7456 | 45.1127 |
| 1-26d | 54.3955 | 0.5664 | 0.0934 | 50.543 | 16.7051 | 47.0098 |
| 1-26e | 36.6369 | 0.7275 | 0.1162 | 70.0149 | 10.7288 | 48.273 |
| 1-36a | 43.9038 | 23.4607 | 0.4003 | 37.8737 | 12.3842 | 43.023 |
| 1-36b | 46.2296 | 17.4734 | 0.2509 | 28.3498 | 17.1992 | 51.7303 |
| 1-36c | 33.5473 | 22.7172 | 0.384 | 32.9889 | 10.9137 | 53.8241 |
| 1-36d | 30.3483 | 21.9108 | 0.3459 | 32.6397 | 12.4463 | 55.6903 |
| 1-36e | 34.2625 | 22.4675 | 0.4111 | 33.3163 | 10.3248 | 53.7075 |
| 1-41a | 50.84 | 0.6102 | 0.1238 | 53.2111 | 12.3563 | 47.0405 |
| 1-41b | 27.0994 | 0.9407 | 0.0998 | 67.672 | 11.9654 | 54.0057 |
| 1-41c | 54.6053 | 0.5387 | 0.1112 | 52.0424 | 14.0354 | 47.3747 |
| 1-41d | 58.9086 | 0.6276 | 0.1701 | 51.36 | 21.0093 | 44.2126 |
| 1-41e | 48.8262 | 0.5743 | 0.1143 | 57.4473 | 13.8819 | 45.8719 |
| 1-40a | 38.7776 | 39.7726 | 0.2401 | 21.5304 | 12.246 | 44.721 |
| 1-40b | 44.7092 | 49.927 | 0.3738 | 22.8583 | 10.4058 | 44.314 |
| 1-40c | 43.6888 | 31.47 | 0.2197 | 29.7888 | 10.3401 | 50.5487 |
| 1-40d | 43.0144 | 32.1802 | 0.1935 | 28.5121 | 13.9717 | 49.4035 |
| 1-40e | 38.1811 | 32.5265 | 0.2004 | 31.8564 | 10.647 | 51.5381 |
| 1-20a | 43.2545 | 0.7591 | 0.0973 | 60.0985 | 13.8425 | 44.7316 |
| 1-20b | 44.7893 | 0.7386 | 0.1007 | 69.3394 | 13.0988 | 46.5606 |
| 1-20c | 29.207 | 0.673 | 0.1105 | 63.3536 | 16.4597 | 49.4451 |
| 1-20d | 39.0349 | 0.612 | 0.1224 | 57.5853 | 14.8878 | 49.7504 |
| 1-20e | 48.061 | 0.7296 | 0.1017 | 61.7944 | 10.9017 | 47.5667 |

### Example 10—Shake Flask Fermentation Assay

Strains 1-13c, 1-30a-e, 1-31a-e, 1-32a-e, 1-4a-e, 1-5a-e, 1-6a-e, 1-7a-e, and 1-9a-e (outlined in Table 4 above), were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

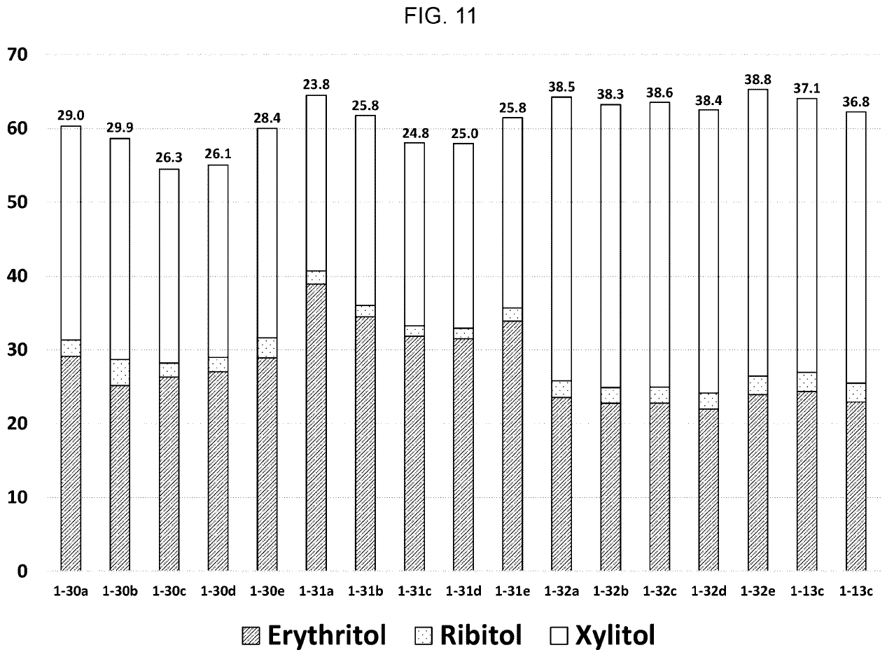

A 250 ml non-baffled flask containing production medium (Table 5) was inoculated with 0.8 mL of the seed culture to form the production culture. The production culture was incubated at 35 °C and 250 rpm. Samples were taken from the production culture after 72 and 96 hours of incubation. Samples were analyzed for glucose, ribitol, xylitol, erythritol, glycerol, and ethanol by high performance liquid chromatography with refractive index detector. Fermentation results are reported in Table 11 and FIG. 11.

While PCR verification indicated that the transformed polynucleotide sequence was present in the indicated strains, further analysis indicated that in some strains, the sequence was not correctly integrated at the ER1 locus. Further analysis indicated that strains 1-30c and 1-30d include the transformed polynucleotide sequence, but it is not integrated at the ER1 locus. Further analysis was inconclusive on the integration locus in strain 1-6c.

TABLE 11
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-13c | 65.2534 | 2.6255 | 37.1061 | 24.3306 | 10.3659 | 40.8187 |
| 1-13c | 63.7146 | 2.5391 | 36.7742 | 22.9396 | 9.9483 | 41.7289 |
| 1-30a | 64.53 | 2.2035 | 28.9854 | 29.1288 | 11.3525 | 39.7976 |
| 1-30b | 58.571 | 3.5146 | 29.9395 | 25.1729 | 12.5878 | 42.2656 |
| 1-30c | 61.7808 | 1.8694 | 26.2541 | 26.3438 | 15.1573 | 43.1151 |
| 1-30d | 57.4209 | 1.9473 | 26.054 | 27.0466 | 13.4411 | 44.8634 |
| 1-30e | 74.7224 | 2.7504 | 28.386 | 28.8879 | 14.2582 | 37.7887 |
| 1-31a | 65.2966 | 1.7919 | 23.8031 | 38.9002 | 11.2516 | 37.7702 |
| 1-31b | 58.0672 | 1.528 | 25.7531 | 34.4798 | 12.434 | 40.5374 |
| 1-31c | 60.9593 | 1.4068 | 24.8131 | 31.8421 | 11.6425 | 41.8413 |
| 1-31d | 60.5991 | 1.4246 | 25.033 | 31.5056 | 11.5117 | 42.3126 |
| 1-31e | 65.0496 | 1.7865 | 25.7844 | 33.8874 | 12.8272 | 40.8807 |
| 1-32a | 67.1283 | 2.236 | 38.4541 | 23.5687 | 11.1949 | 38.707 |
| 1-32b | 70.2815 | 2.1497 | 38.3141 | 22.7553 | 10.5468 | 38.8618 |
| 1-32c | 68.0248 | 2.2063 | 38.5515 | 22.7602 | 10.3843 | 38.5577 |
| 1-32d | 66.3494 | 2.1502 | 38.3931 | 21.9976 | 11.0603 | 40.7174 |
| 1-32e | 63.8648 | 2.5133 | 38.8442 | 23.9287 | 10.8515 | 39.9071 |
| 1-4a | 70.2783 | 0.4834 | 0.3673 | 46.1316 | 14.32 | 43.4293 |
| 1-4b | 72.2855 | 0.7763 | 0.1831 | 57.21 | 19.3193 | 35.128 |
| 1-4c | 56.1849 | 0.5101 | 0.1291 | 47.8537 | 15.7451 | 47.9659 |
| 1-4d | 77.2722 | 0.5838 | 0.1901 | 50.3863 | 14.1389 | 39.9826 |
| 1-4e | 70.9407 | 0.4671 | 0.1525 | 46.2662 | 15.3398 | 44.2176 |
| 1-5a | 56.7359 | 0.6505 | 0.1281 | 69.7951 | 13.2652 | 40.3786 |
| 1-5b | 66.4853 | 0.4758 | 0.1138 | 47.5548 | 17.2347 | 44.8984 |
| 1-5c | 56.2946 | 0.4561 | 0.1072 | 46.0599 | 15.9891 | 48.8712 |
| 1-5d | 63.8624 | 0.5188 | 0.1223 | 55.0207 | 15.5579 | 41.5538 |
| 1-5e | 53.5706 | 0.4438 | 0.0978 | 46.4767 | 15.7478 | 49.9892 |
| 1-6a | 53.5487 | 0.4466 | 0.0857 | 46.0621 | 14.3516 | 50.2479 |
| 1-6b | 56.3098 | 0.5689 | 0.0987 | 59.4119 | 14.2449 | 44.3129 |
| 1-6c | 62.2098 | 0.4975 | 0.1159 | 50.7104 | 13.912 | 46.5117 |
| 1-6d | 88.3493 | 0.392 | 0.0963 | 46.7061 | 15.7906 | 39.0682 |
| 1-6e | 66.3903 | 0.513 | 0.1236 | 54.7569 | 15.6941 | 42.6935 |
| 1-7a | 84.3102 | 0.7204 | 0.0924 | 52.2635 | 19.827 | 33.7597 |
| 1-7b | 69.0519 | 0.4897 | 0.1187 | 51.9057 | 14.6164 | 43.9185 |
| 1-7c | 67.7409 | 0.5193 | 0.1426 | 53.5386 | 14.7419 | 42.8362 |
| 1-7d | 61.1123 | 0.5279 | 0.1161 | 54.4933 | 14.502 | 43.378 |
| 1-7e | 58.0693 | 0.4778 | 0.1356 | 50.1206 | 18.9325 | 46.1013 |
| 1-9a | 52.6189 | 0.5986 | 0.1156 | 66.6905 | 13.1314 | 43.3405 |
| 1-9b | 59.3034 | 0.464 | 0.1102 | 58.2455 | 14.7562 | 43.3825 |
| 1-9c | 53.516 | 0.4302 | 0.1246 | 51.9753 | 15.0032 | 48.9462 |
| 1-9d | 53.9501 | 0.4221 | 0.0876 | 46.6648 | 15.398 | 49.7159 |
| 1-9e | 54.575 | 0.4136 | 0.0957 | 46.5254 | 15.3139 | 49.828 |

### Example 11—Two Copy XPDH Modified Moniliella pollinis Strains

Strain 1-1 was transformed as described in Example 4 with SEQ ID NO:55 and SEQ ID NO: 177. SEQ ID NO:177 contains (i) a 3′ portion of the G418 selectable marker (SEQ ID NO: 172); ii) an expression cassette for the XPDH homolog from Clostridium difficile, SEQ ID NO: 98 encoding the amino acid sequence of SEQ ID NO:12, under the control of the PYK1 promoter of SEQ ID NO:86 and the PGD terminator of SEQ ID NO:140; and (iii) a 3′ flanking DNA for targeted chromosomal integration into the ER1 locus (SEQ ID NO:162). SEQ ID NO:55 contains i) a 5′ flanking DNA for targeted chromosomal integration into the ER1 locus (SEQ ID NO: 85), ii) an expression cassette for the XPDH homolog from Clostridium difficile, SEQ ID NO: 98 encoding the amino acid sequence of SEQ ID NO:12, under the control of the PYK1 promoter of SEQ ID NO:86 and the PGD terminator of SEQ ID NO:140; and (iii) a 5′ portion of the G418 selectable marker (SEQ ID NO:175). Transformants were selected on PDA+250 mg/L geneticin (G418) selection plates and incubated at 35 °C for at least 2 days until transformants grow. Resulting transformants were streaked for single colony isolation on PDA+geneticin (G418) plates and single colonies were selected. Selected colonies were evaluated by colony PCR for integration of two copies of the C. difficile XPDH sequence. PCR verified isolates were designated strains 2-1a, 2-1b, 2-1c, 2-1d, and 2-1e.

Strain 1-1 was transformed as described in Example 4 with SEQ ID NO:57 and SEQ ID NO: 178. SEQ ID NO:178 contains (i) a 3′ portion of the G418 selectable marker (SEQ ID NO: 172); ii) an expression cassette for the XPDH homolog from Lactobacillus rhamnosus, SEQ ID NO: 100 encoding the amino acid sequence of SEQ ID NO:14, under the control of the PYK1 promoter of SEQ ID NO:86 and the PGD terminator of SEQ ID NO:140; and (iii) a 3′ flanking DNA for targeted chromosomal integration into the ER1 locus (SEQ ID NO:162). SEQ ID NO:57 contains (i) 5′ flanking DNA for targeted chromosomal integration into the ER1 locus (SEQ ID NO: 85); (ii) an expression cassette for the XPDH homolog from Lactobacillus rhamnosus, SEQ ID NO: 100 encoding the amino acid sequence of SEQ ID NO:14, under the control of the PYK1 promoter of SEQ ID NO:86 and the PGD terminator of SEQ ID NO:140; and (iii) a 5′ portion of the G418 selectable marker (SEQ ID NO:175). Transformants were selected on PDA+250 mg/L geneticin (G418) selection plates and incubated at 35 °C for at least 2 days until transformants grow. Resulting transformants were streaked for single colony isolation on PDA+geneticin (G418) plates and single colonies were selected. Selected colonies were evaluated by colony PCR for integration of two copies of the L. rhamnosus XPDH sequence. PCR verified isolates were designated strains 2-2a, 2-2b, 2-2c, 2-2d, and 2-2e.

Strains 1-13c, 2-1a-e, and 2-2a-e were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

A 250 ml non-baffled flask containing production medium (Table 5) was inoculated with 0.8 mL of the seed culture to form the production culture. The production culture was incubated at 35 °C and 250 rpm. Samples were taken from the production culture after 72 and 96 hours of incubation. Samples were analyzed for glucose, ribitol, xylitol, erythritol, glycerol, and ethanol by high performance liquid chromatography with refractive index detector. Fermentation results are reported in Table 12.

While PCR verification indicated that the transformed polynucleotide sequence was present in the indicated strains, further analysis indicated that in some strains, the sequence was not correctly targeted to the ER1 locus. Further analysis indicated that strain 2-2e includes the transformed polynucleotide sequence, but it is not targeted to the ER1 locus.

TABLE 12
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-13c | 104.7609 | 2.4041 | 25.3431 | 20.3872 | 8.3483 | 28.8597 |
| 2-1a | 99.6084 | 3.3114 | 27.2743 | 20.3414 | 8.0997 | 31.7018 |
| 2-1b | 101.9287 | 3.3201 | 27.2884 | 20.247 | 8.2186 | 32.2224 |
| 2-1c | 86.0682 | 2.0223 | 31.4376 | 18.3715 | 9.4971 | 36.504 |
| 2-1d | 111.1154 | 3.3146 | 23.8856 | 20.3704 | 8.1327 | 30.6248 |
| 2-1e | 88.2859 | 3.6673 | 29.2664 | 21.0497 | 8.3223 | 27.8294 |
| 2-2a | 90.298 | 10.4088 | 25.7466 | 18.7038 | 8.2613 | 33.5813 |
| 2-2b | 79.873 | 11.4485 | 27.4826 | 23.3037 | 9.5481 | 34.5981 |
| 2-2c | 102.0879 | 19.1571 | 17.6362 | 13.2534 | 9.1421 | 31.8373 |
| 2-2d | 103.634 | 9.8275 | 25.1036 | 18.2777 | 7.1005 | 32.0808 |
| 2-2e | 80.9998 | 14.2648 | 19.9443 | 22.9617 | 7.6286 | 31.3374 |

### Example 12—Genetically Modified Moniliella pollinis Strains

Strain 1-1 was transformed with SEQ ID NO: 186 and SEQ ID NO: 187 as described in Example 4. SEQ ID NO:186 contained (i) 5′ flanking DNA for targeted chromosomal integration into the ER1 locus (SEQ ID NO:85), (ii) an MpPYK1 promoter (SEQ ID NO:86), (iii) a gene encoding the Staphylococcus aureus xylitol dehydrogenase of SEQ ID NO:34, (iv) an Mp6PGD terminator (SEQ ID NO:140), and (v) a 5′ portion of a G418 resistance gene expression cassette (SEQ ID NO:175). SEQ ID NO: 187 contained (i) a 3′ portion of a G418 resistance gene expression cassette (SEQ ID NO: 172), (ii) an MpTEF2 terminator (SEQ ID NO:150), (iii) an MpPGK1 promoter (SEQ ID NO:135), (iv) a gene encoding the Saccharomyces cerevisiae DOG1 sugar phosphatase of SEQ ID NO:188, (v) an MpENO1 terminator (SEQ ID NO:142), and (vi) 3′ flanking DNA for targeted chromosomal integration into the ER1 locus (SEQ ID NO: 162). Transformants were selected on PDA+250 mg/L geneticin (G418) selection plates and incubated at 35 °C for at least 2 days until transformants grow. Resulting transformants were streaked for single colony isolation on PDA+geneticin (G418) plates and single colonies were selected. Selected colonies were evaluated by colony PCR for integration of the indicated sequence. A PCR verified isolate were designated strain 3-1.

Table 13 below lists various Moniliella pollinis strains, including information on the parent strain, the sequence with which the parent strain was transformed, and characterizations of the expression cassette(s) contained on the transformed sequence. The transformation fragment of SEQ ID NO:181 contained, in order, a 5′ ER3 flanking sequence (SEQ ID NO:155), an MpPYK1 promoter (SEQ ID NO:86), a gene encoding the M. pollinis RPE2 polypeptide of SEQ ID NO:180, an MpPYK1 terminator (SEQ ID NO:146), and a 5′ portion of a zeocin resistance gene expression cassette (SEQ ID NO:169). The transformation fragment of SEQ ID NO:182 contained, in order, a 3′ portion of a zeocin resistance gene expression cassette (SEQ ID NO:168), an MpTEF2 terminator (SEQ ID NO:150), and a 3′ ER3 flanking sequence (SEQ ID NO:165). The transformation fragment of SEQ ID NO:183 contained, in order, a 3′ portion of a zeocin resistance gene expression cassette (SEQ ID NO:168), an Mp6PGD promoter (SEQ ID NO:130), a gene encoding the M. pollinis RPE1 polypeptide of SEQ ID NO: 179, an Mp6PGD terminator (SEQ ID NO: 140), and a 3′ER3 flanking sequence (SEQ ID NO: 165). The transformation fragment of SEQ ID NO: 184 contained, in order, a 3′ portion of a zeocin resistance gene expression cassette (SEQ ID NO: 168), an Mp6PGD promoter (SEQ ID NO:130), a gene encoding the M. pollinis RPE2 polypeptide od SEQ ID NO: 180, an Mp6PDG terminator (SEQ ID NO:140), and a 3′ER3 flanking sequence (SEQ ID NO: 165). The transformation fragment of SEQ ID NO:185 contained, in order, a 5′ ER3 flanking sequence (SEQ ID NO:155), an MpTEF1 promoter (SEQ ID NO:133), and a 5′ portion of a zeocin resistance gene expression cassette (SEQ ID NO:169).

The indicated Moniliella pollinis parent strain was transformed with the indicated sequence(s) as described in Example 4 using either zeocin or G418 selection corresponding to the indicated selection marker. Resulting transformants were evaluated by colony PCR for integration of the indicated sequence. A PCR verified isolate was then designated as the indicated strain number. In some instances, more than one PCR verified isolate, e.g., “sister” isolates, are indicated by letters following the strain number. For example, strain 3-2 has 4 sister isolates, strains 3-2a, 3-2b, 3-2c, and 3-2d (collectively 3-2a-d).

TABLE 13

| | | First Bipartite Cassette | | Second Bipartite Cassette | |
|---|---|---|---|---|---|
| | | Transfor- | Encoded | Transfor- | Encoded |
| | Parent | mation | Polypeptide | mation | Polypeptide |
| Strain | Strain | SEQ ID NO: | SEQ ID NO: | SEQ ID NO: | SEQ ID NO: |
| 3-2a-c | 3-1 | 181 | 180 | 182 | na |
| 3-3a-c | 3-1 | 183 | 180 | 185 | na |
| 3-4a-c | 3-1 | 184 | 179 | 185 | na |
| 3-5a-c | 3-1 | 181 | 180 | 183 | 179 |
| 3-6a-c | 3-1 | 181 | 180 | 184 | 179 |
| 3-7a-f | 1-13c | 181 | 180 | 182 | na |
| 3-8a-f | 1-15a | 181 | 180 | 182 | na |
| 3-9a-d | 1-29c | 181 | 180 | 182 | na |
| 3-10a-e | 1-34d | 181 | 180 | 182 | na |
| 3-11a-e | 2-1c | 181 | 180 | 182 | na |
| 3-12a-e | 2-2b | 181 | 180 | 182 | na |
| 3-13a-e | 1-30b | 181 | 180 | 182 | na |
| 3-14a-e | 1-31d | 181 | 180 | 182 | na |
| 3-15a-e | 1-32d | 181 | 180 | 182 | na |
| 3-16a-e | 1-14d | 181 | 180 | 182 | na |
| 3-17a-e | 1-16a | 181 | 180 | 182 | na |

na-not applicable, second cassette used for recombination and selection marker only

### Example 13—Shake Flask Fermentation Assay

Strains 1-1, 3-1, 3-2a-c, 3-3a-c, 3-4a-c, 3-5a-c, and 3-6a-c were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

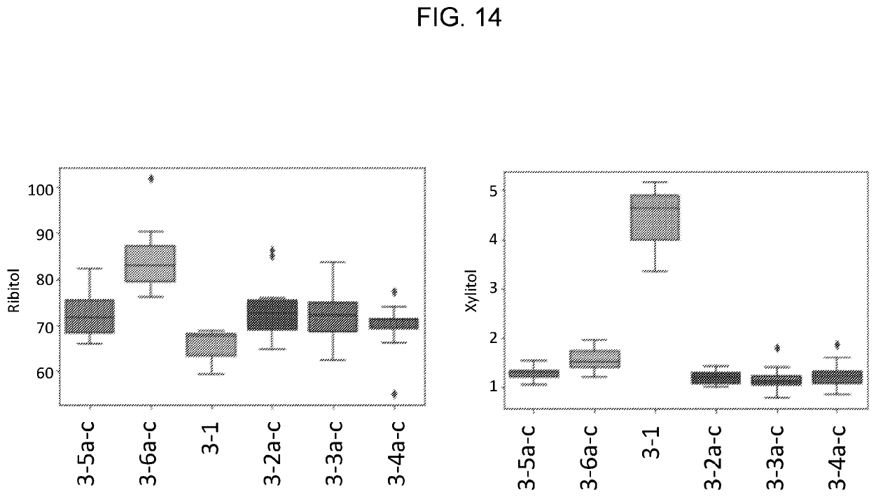

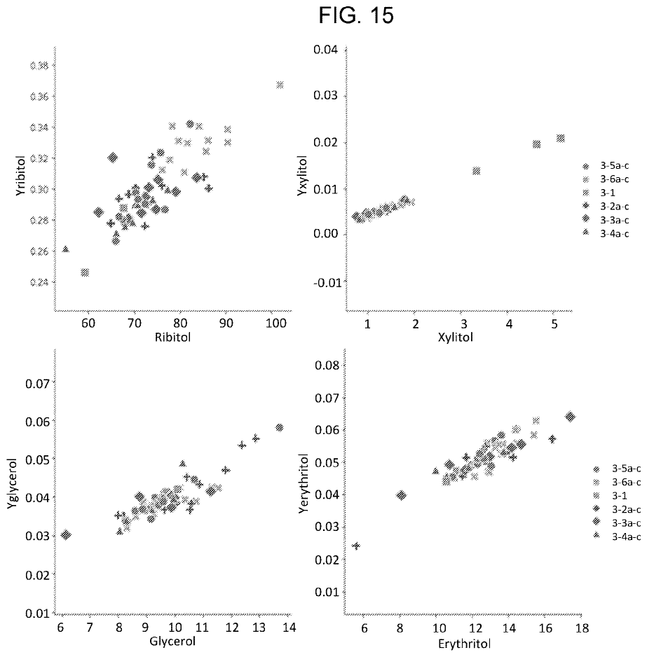

A 250 ml non-baffled flask containing production medium (Table 5) was inoculated with 0.8 mL of the seed culture to form the production culture. The production culture was incubated at 35 °C and 250 rpm. Samples were taken from the production culture after 48, 72, and 96 hours of incubation. Samples were analyzed for glucose, ribitol, xylitol, erythritol, glycerol, and ethanol by high performance liquid chromatography with refractive index detector. Fermentation results are reported in Table 14 and FIGS. 14 and 15.

TABLE 14
96 Hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 3-5c | 40.9 | 82.2 | 1.5 | 14.5 | 9.6 | 40.8 |
| 3-6a | 14.3 | 90.4 | 1.8 | 12.1 | 11.3 | 39.7 |
| 3-6b | 7.5 | 90.4 | 1.7 | 12.9 | 11.6 | 40.6 |
| 3-6c | 4.0 | 101.9 | 1.9 | 13.0 | 10.8 | 40.1 |
| 3-1 | 46.5 | 67.6 | 4.6 | 11.1 | 9.7 | 44.3 |
| 3-1 | 34.9 | 68.7 | 5.2 | 11.5 | 9.9 | 41.0 |
| 3-2a | 48.2 | 76.0 | 1.3 | 11.5 | 11.8 | 39.3 |
| 3-2b | 23.3 | 85.1 | 1.3 | 14.2 | 10.6 | 43.3 |
| 3-2c | 12.5 | 86.2 | 1.4 | 16.4 | 10.5 | 50.6 |
| 3-3a | 38.3 | 74.9 | 1.2 | 14.2 | 10.0 | 47.4 |
| 3-3b | 34.0 | 79.1 | 1.3 | 14.7 | 9.9 | 47.0 |
| 3-3c | 26.9 | 83.7 | 1.1 | 17.4 | 11.3 | 46.3 |
| 3-4a | 41.4 | 77.4 | 1.6 | 13.7 | 8.1 | 43.4 |
| 3-4b | 46.9 | 74.1 | 1.3 | 12.7 | 10.0 | 42.4 |
| 3-4c | 57.2 | 70.2 | 1.2 | 12.1 | 9.0 | 43.3 |
| 1-1 | 97.9 | 0.9 | 0.1 | 53.2 | 13.7 | 31.7 |
| 3-1 | 58.8 | 59.3 | 3.3 | 10.6 | 10.1 | 45.3 |

### Example 14—Shake Flask Fermentation Assay

Strains 1-1, 1-13c, 1-15a, 3-7a-f, and 3-8a-f, were run in shake flasks to assess glucose consumption as well as ribitol, xylitol, glycerol, erythritol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

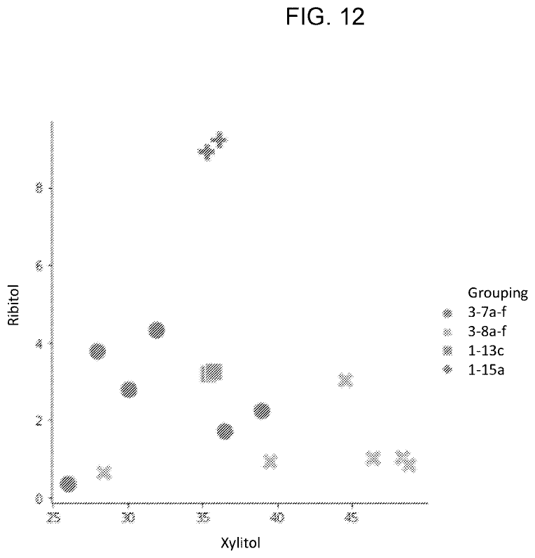

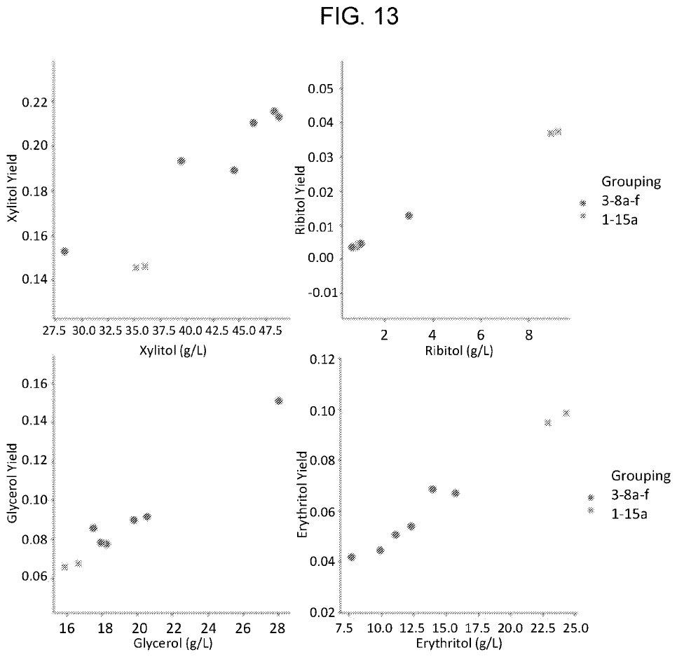

A 250 ml non-baffled flask containing production medium (Table 5) was inoculated with 0.8 mL of the seed culture to form the production culture. The production culture was incubated at 35 °C and 250 rpm. Samples were taken from the production culture after 48, 72, and 96 hours of incubation. Samples were analyzed for glucose, ribitol, xylitol, erythritol, glycerol, and ethanol by high performance liquid chromatography with refractive index detector. Fermentation results are reported in Table 15 and FIGS. 12 and 13.

TABLE 15
96 Hour Shake Flask Results

| Strain | Glucose | Ribitol | Xylitol | Erythritol | Glycerol | Ethanol |
|---|---|---|---|---|---|---|
| 3-7a | 137.81 | 0.35 | 26.02 | 11.69 | 23.16 | 21.44 |
| 3-7b | 59.81 | 2.78 | 30.09 | 24.53 | 21.77 | 37.56 |
| 3-7c | 61.19 | 2.23 | 38.98 | 21.32 | 16.97 | 36.23 |
| 3-7d | 81.93 | 1.70 | 36.50 | 19.87 | 20.79 | 24.54 |
| 3-7e | 60.27 | 4.31 | 31.94 | 23.23 | 16.40 | 35.79 |
| 3-7f | 73.27 | 3.77 | 27.98 | 32.10 | 17.63 | 28.82 |
| 3-8a | 66.88 | 3.01 | 44.57 | 15.77 | 18.28 | 33.11 |
| 3-8b | 77.84 | 1.03 | 48.40 | 9.95 | 20.58 | 30.25 |
| 3-8c | 97.96 | 0.93 | 39.53 | 14.00 | 17.55 | 26.02 |
| 3-8d | 73.16 | 0.84 | 48.83 | 12.32 | 17.96 | 34.21 |
| 3-8e | 116.78 | 0.65 | 28.41 | 7.72 | 28.04 | 24.53 |
| 3-8f | 81.82 | 1.01 | 46.42 | 11.13 | 19.83 | 31.73 |
| 1-13c | 60.56 | 3.20 | 35.39 | 25.66 | 16.35 | 38.60 |
| 1-13c | 59.43 | 3.27 | 35.70 | 26.11 | 16.39 | 37.46 |
| 1-15a | 55.73 | 9.23 | 36.08 | 24.34 | 16.69 | 35.93 |
| 1-15a | 60.66 | 8.93 | 35.24 | 22.91 | 15.91 | 37.51 |
| 1-1 | 55.56 | 0.63 | 0.33 | 59.10 | 15.20 | 43.83 |
| 1-1 | 57.65 | 0.59 | 0.09 | 58.06 | 15.22 | 44.85 |

### Example 15—Shake Flask Fermentation Assay

Strains 1-13c, 1-29c, 1-34d, 1-30b, 1-31d, 1-32d, 1-14d, 1-16a, 2-1c, 2-2b, 3-8b, 3-9a-d, 3-10a-e, 3-11a-e, 3-12a-e, 3-13a-e, 3-14a-e, 3-15a-e, 3-16a-e, and 3-17a-e were run in shake flasks to assess glucose consumption as well as ribitol, xylitol, glycerol, erythritol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

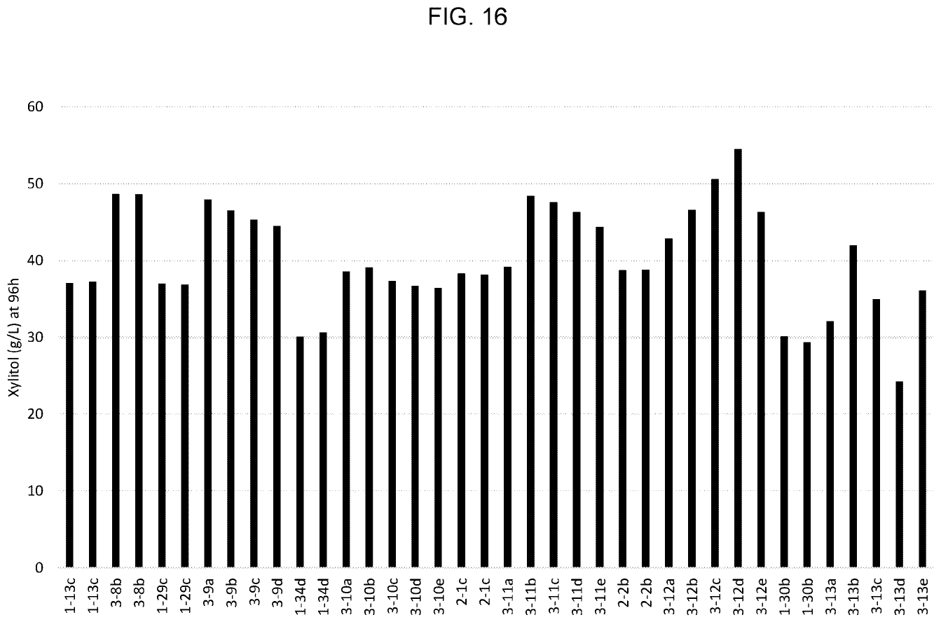

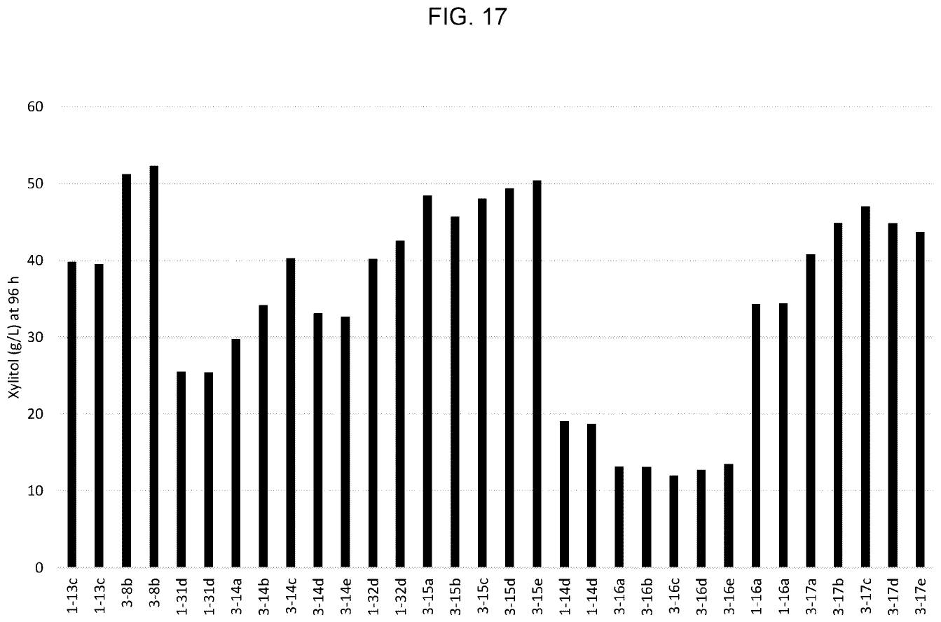

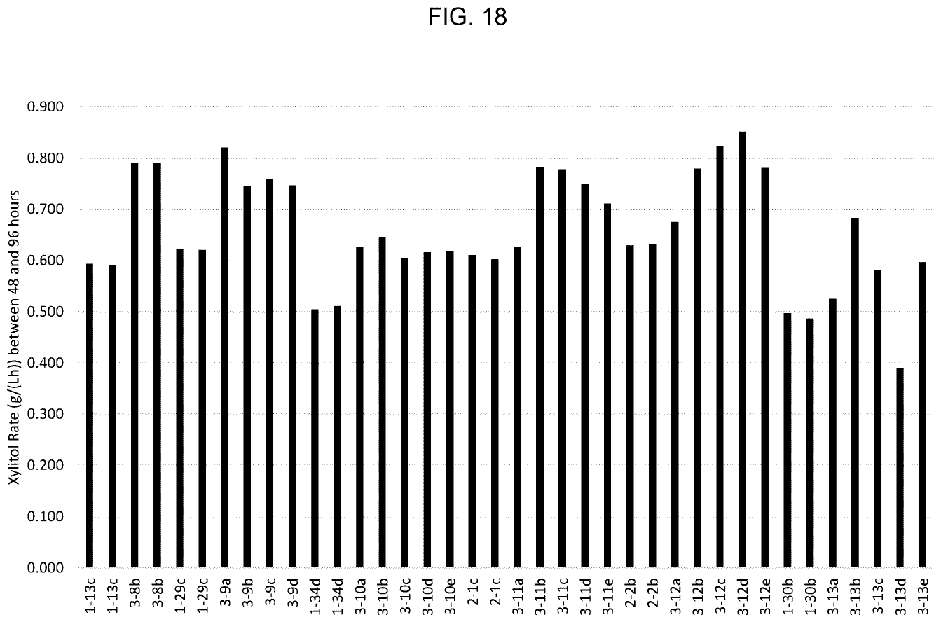

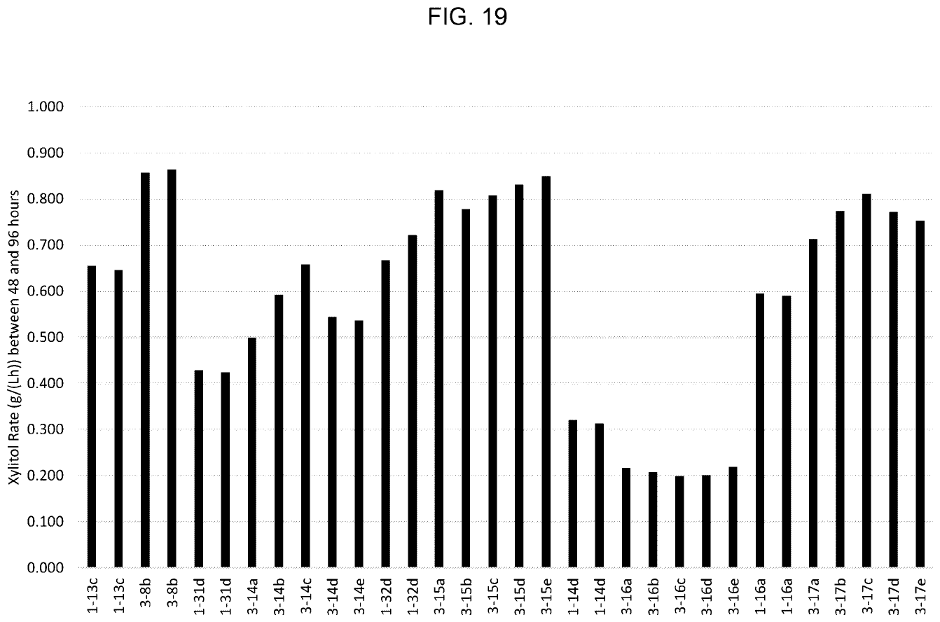

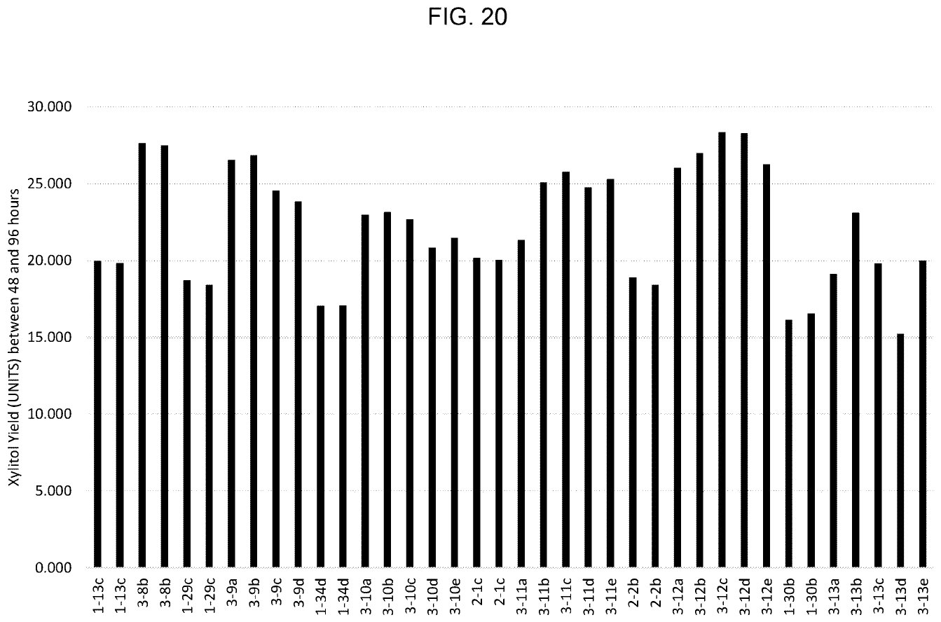

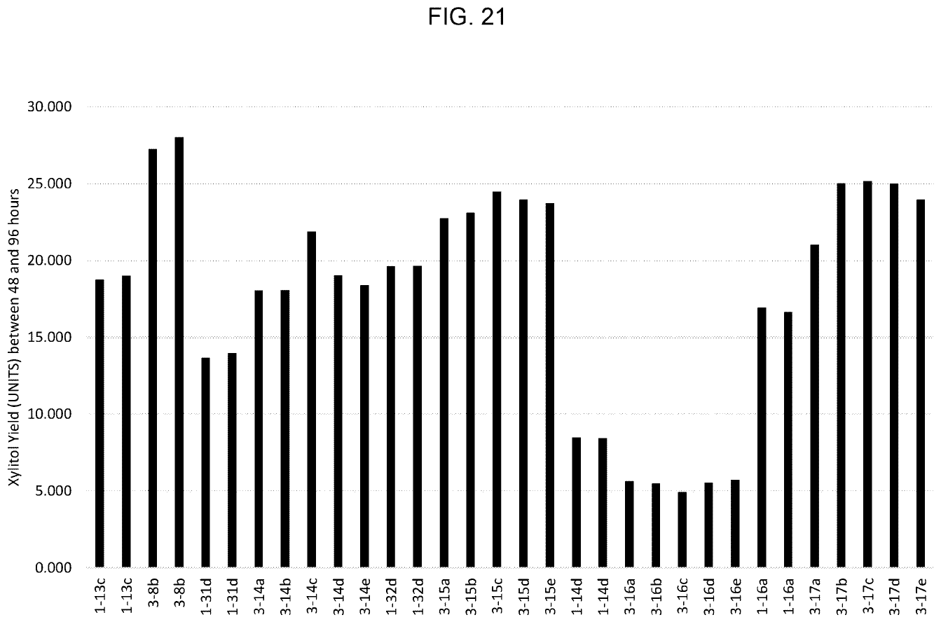

A 250 ml non-baffled flask containing production medium (Table 5) was inoculated with 0.8 mL of the seed culture to form the production culture. The production culture was incubated at 35 °C and 250 rpm. Samples were taken from the production culture after 48, 72, and 96 hours of incubation. Samples were analyzed for glucose, ribitol, xylitol, erythritol, glycerol, and ethanol by high performance liquid chromatography with refractive index detector. Fermentation results are reported in Tables 16-18 and FIGS. 16-21.

Yield was calculated as the ratio of product (i.e., xylitol) to consumed substrate (i.e., the difference between one time point and a second time point) expressed as a percentage.

TABLE 16
Shake Flask Yield (48-96 hours)

| | Yield (%) | | | | | Yield (%) | | | |
|---|---|---|---|---|---|---|---|---|---|
| Strain | Xylitol | Erythritol | Glycerol | Ethanol | Strain | Xylitol | Erythritol | Glycerol | Ethanol |
| 1-13c | 19.959 | 11.186 | 2.257 | 13.580 | 3-11b | 25.080 | 6.166 | 2.468 | 13.926 |
| 1-13c | 19.814 | 11.414 | 2.216 | 13.050 | 3-11c | 25.760 | 5.215 | 2.673 | 13.881 |
| 3-8b | 27.629 | 4.687 | 4.050 | 14.679 | 3-11d | 24.767 | 5.083 | 2.543 | 15.108 |
| 3-8b | 27.481 | 4.595 | 3.971 | 14.442 | 3-11e | 25.289 | 4.599 | 3.816 | 14.623 |
| 1-29c | 18.720 | 12.282 | 2.377 | 14.249 | 2-2b | 18.910 | 11.461 | 2.201 | 13.718 |
| 1-29c | 18.404 | 12.671 | 2.775 | 12.142 | 2-2b | 18.408 | 11.566 | 2.255 | 12.415 |
| 3-9a | 26.535 | 7.925 | 3.461 | 13.868 | 3-12a | 26.020 | 5.583 | 1.943 | 12.034 |
| 3-9b | 26.826 | 3.929 | 5.377 | 14.966 | 3-12b | 26.977 | 5.511 | 3.627 | 15.343 |
| 3-9c | 24.530 | 5.946 | 2.640 | 16.006 | 3-12c | 28.343 | 6.408 | 2.902 | 14.361 |
| 3-9d | 23.828 | 5.807 | 2.828 | 16.210 | 3-12d | 28.267 | 5.617 | 2.755 | 13.960 |
| 1-34d | 17.041 | 13.390 | 2.302 | 13.848 | 3-12e | 26.270 | 5.249 | 3.386 | 15.419 |
| 1-34d | 17.072 | 14.176 | 2.401 | 13.291 | 1-30b | 16.127 | 10.271 | 2.076 | 12.980 |
| 3-10a | 22.964 | 8.298 | 2.574 | 13.900 | 1-30b | 16.544 | 10.207 | 2.166 | 15.502 |
| 3-10b | 23.146 | 8.036 | 2.385 | 14.188 | 3-13a | 19.117 | 6.541 | 2.854 | 15.612 |
| 3-10c | 22.684 | 4.942 | 2.511 | 15.314 | 3-13b | 23.099 | 4.694 | 2.639 | 16.391 |
| 3-10d | 20.829 | 11.467 | 2.653 | 13.903 | 3-13c | 19.796 | 5.038 | 2.815 | 15.925 |
| 3-10e | 21.474 | 8.567 | 3.162 | 15.026 | 3-13d | 15.223 | 10.518 | 2.866 | 13.613 |
| 2-1c | 20.166 | 9.682 | 2.500 | 14.607 | 3-13e | 19.989 | 6.261 | 2.196 | 16.335 |
| 2-1c | 20.024 | 9.760 | 2.384 | 15.034 | 1-13c | 18.750 | 11.359 | 2.003 | 13.347 |
| 3-11a | 21.329 | 3.619 | 3.540 | 17.541 | 1-13c | 19.001 | 11.261 | 1.884 | 13.125 |
| 1-31d | 13.660 | 15.694 | 2.300 | 13.408 | 1-14d | 8.481 | 5.035 | 0.509 | 13.569 |
| 1-31d | 13.956 | 14.953 | 2.251 | 14.761 | 1-14d | 8.432 | 5.020 | 0.539 | 13.947 |
| 3-14a | 18.058 | 9.612 | 2.787 | 14.067 | 3-16a | 5.631 | 4.911 | 0.335 | 14.403 |
| 3-14b | 18.068 | 8.387 | 2.544 | 14.029 | 3-16b | 5.498 | 4.755 | 0.424 | 13.935 |
| 3-14c | 21.889 | 7.983 | 3.006 | 14.062 | 3-16c | 4.917 | 5.181 | 0.544 | 14.352 |
| 3-14d | 19.018 | 8.169 | 2.921 | 12.995 | 3-16d | 5.530 | 4.944 | 1.117 | 13.925 |
| 3-14e | 18.381 | 8.134 | 2.943 | 13.879 | 3-16e | 5.746 | 5.071 | 0.366 | 13.030 |
| 1-32d | 19.642 | 9.419 | 1.944 | 14.914 | 1-16a | 16.916 | 13.856 | 2.115 | 15.615 |
| 1-32d | 19.660 | 12.618 | 2.311 | 10.942 | 1-16a | 16.658 | 14.100 | 2.135 | 15.424 |
| 3-15a | 22.748 | 4.900 | 2.841 | 17.310 | 3-17a | 21.029 | 9.191 | 2.294 | 14.448 |
| 3-15b | 23.106 | 6.824 | 2.176 | 15.358 | 3-17b | 25.018 | 8.711 | 2.193 | 13.334 |
| 3-15c | 24.477 | 5.354 | 1.934 | 15.954 | 3-17c | 25.159 | 7.043 | 2.328 | 14.155 |
| 3-15d | 23.977 | 4.755 | 1.929 | 17.373 | 3-17d | 24.997 | 7.345 | 2.053 | 15.495 |
| 3-15e | 23.732 | 6.352 | 2.116 | 13.950 | 3-17e | 23.978 | 6.848 | 2.443 | 16.012 |

TABLE 17
Shake Flask Rate (48-96 hours)

| | Rate (g/(L · h)) | | | | | | Rate (g/(L · h)) | | | | |
|---|---|---|---|---|---|---|---|---|---|---|---|
| Strain | Glucose | Xylitol | Erythritol | Glycerol | Ethanol | Strain | Glucose | Xylitol | Erythritol | Glycerol | Ethanol |
| 1-13c | 2.972 | 0.593 | 0.332 | 0.067 | 0.404 | 3-10b | 2.792 | 0.646 | 0.224 | 0.067 | 0.396 |
| 1-13c | 2.987 | 0.592 | 0.341 | 0.066 | 0.390 | 3-10c | 2.667 | 0.605 | 0.132 | 0.067 | 0.408 |
| 3-8b | 2.859 | 0.790 | 0.134 | 0.116 | 0.420 | 3-10d | 2.957 | 0.616 | 0.339 | 0.078 | 0.411 |
| 3-8b | 2.878 | 0.791 | 0.132 | 0.114 | 0.416 | 3-10e | 2.876 | 0.618 | 0.246 | 0.091 | 0.432 |
| 1-29c | 3.325 | 0.622 | 0.408 | 0.079 | 0.474 | 2-1c | 3.030 | 0.611 | 0.293 | 0.076 | 0.443 |
| 1-29c | 3.370 | 0.620 | 0.427 | 0.093 | 0.409 | 2-1c | 3.008 | 0.602 | 0.294 | 0.072 | 0.452 |
| 3-9a | 3.093 | 0.821 | 0.245 | 0.107 | 0.429 | 3-11a | 2.935 | 0.626 | 0.106 | 0.104 | 0.515 |
| 3-9b | 2.781 | 0.746 | 0.109 | 0.150 | 0.416 | 3-11b | 3.119 | 0.782 | 0.192 | 0.077 | 0.434 |
| 3-9c | 3.094 | 0.759 | 0.184 | 0.082 | 0.495 | 3-11c | 3.020 | 0.778 | 0.158 | 0.081 | 0.419 |
| 3-9d | 3.135 | 0.747 | 0.182 | 0.089 | 0.508 | 3-11d | 3.024 | 0.749 | 0.154 | 0.077 | 0.457 |
| 1-34d | 2.960 | 0.504 | 0.396 | 0.068 | 0.410 | 3-11e | 2.811 | 0.711 | 0.129 | 0.107 | 0.411 |
| 1-34d | 2.992 | 0.511 | 0.424 | 0.072 | 0.398 | 2-2b | 3.332 | 0.630 | 0.382 | 0.073 | 0.457 |
| 3-10a | 2.722 | 0.625 | 0.226 | 0.070 | 0.378 | 2-2b | 3.431 | 0.631 | 0.397 | 0.077 | 0.426 |
| 3-12a | 2.596 | 0.675 | 0.145 | 0.050 | 0.312 | 1-32d | 3.397 | 0.667 | 0.320 | 0.066 | 0.507 |
| 3-12b | 2.888 | 0.779 | 0.159 | 0.105 | 0.443 | 1-32d | 3.670 | 0.721 | 0.463 | 0.085 | 0.402 |
| 3-12c | 2.905 | 0.823 | 0.186 | 0.084 | 0.417 | 3-15a | 3.600 | 0.819 | 0.176 | 0.102 | 0.623 |
| 3-12d | 3.012 | 0.851 | 0.169 | 0.083 | 0.420 | 3-15b | 3.368 | 0.778 | 0.230 | 0.073 | 0.517 |
| 3-12e | 2.972 | 0.781 | 0.156 | 0.101 | 0.458 | 3-15c | 3.299 | 0.808 | 0.177 | 0.064 | 0.526 |
| 1-30b | 3.081 | 0.497 | 0.316 | 0.064 | 0.400 | 3-15d | 3.468 | 0.831 | 0.165 | 0.067 | 0.602 |
| 1-30b | 2.939 | 0.486 | 0.300 | 0.064 | 0.456 | 3-15e | 3.581 | 0.850 | 0.227 | 0.076 | 0.500 |
| 3-13a | 2.748 | 0.525 | 0.180 | 0.078 | 0.429 | 1-14d | 3.778 | 0.320 | 0.190 | 0.019 | 0.513 |
| 3-13b | 2.958 | 0.683 | 0.139 | 0.078 | 0.485 | 1-14d | 3.708 | 0.313 | 0.186 | 0.020 | 0.517 |
| 3-13c | 2.940 | 0.582 | 0.148 | 0.083 | 0.468 | 3-16a | 3.848 | 0.217 | 0.189 | 0.013 | 0.554 |
| 3-13d | 2.560 | 0.390 | 0.269 | 0.073 | 0.348 | 3-16b | 3.769 | 0.207 | 0.179 | 0.016 | 0.525 |
| 3-13e | 2.983 | 0.596 | 0.187 | 0.066 | 0.487 | 3-16c | 4.050 | 0.199 | 0.210 | 0.022 | 0.581 |
| 1-13c | 3.494 | 0.655 | 0.397 | 0.070 | 0.466 | 3-16d | 3.637 | 0.201 | 0.180 | 0.041 | 0.506 |
| 1-13c | 3.400 | 0.646 | 0.383 | 0.064 | 0.446 | 3-16e | 3.810 | 0.219 | 0.193 | 0.014 | 0.496 |
| 1-31d | 3.137 | 0.429 | 0.492 | 0.072 | 0.421 | 1-16a | 3.514 | 0.594 | 0.487 | 0.074 | 0.549 |
| 1-31d | 3.041 | 0.424 | 0.455 | 0.068 | 0.449 | 1-16a | 3.541 | 0.590 | 0.499 | 0.076 | 0.546 |
| 3-14a | 2.764 | 0.499 | 0.266 | 0.077 | 0.389 | 3-17a | 3.390 | 0.713 | 0.312 | 0.078 | 0.490 |
| 3-14b | 3.275 | 0.592 | 0.275 | 0.083 | 0.459 | 3-17b | 3.095 | 0.774 | 0.270 | 0.068 | 0.413 |
| 3-14c | 3.004 | 0.658 | 0.240 | 0.090 | 0.422 | 3-17c | 3.224 | 0.811 | 0.227 | 0.075 | 0.456 |
| 3-14d | 2.860 | 0.544 | 0.234 | 0.084 | 0.372 | 3-17d | 3.086 | 0.771 | 0.227 | 0.063 | 0.478 |
| 3-14e | 2.919 | 0.537 | 0.237 | 0.086 | 0.405 | 3-17e | 3.143 | 0.754 | 0.215 | 0.077 | 0.503 |

TABLE 18
Shake Flask Results

| | Time | | | | | | | Time | | | | | | |
|---|---|---|---|---|---|---|---|---|---|---|---|---|---|---|
| | Point | Fermentation Broth Analyte (g/L) | | | | | | Point | Fermentation Broth Analyte (g/L) | | | | | |
| Strain | (h) | Glucose | Ribitol | Xylitol | Erythritol | Glycerol | Ethanol | (h) | Glucose | Ribitol | Xylitol | Erythritol | Glycerol | Ethanol |
| 1-13c | 48 | 206.1001 | 0.521 | 8.5702 | 8.4691 | 7.7175 | 19.8144 | 72 | 134.914 | 1.6009 | 20.1782 | 18.1271 | 9.5457 | 34.0047 |
| 1-13c | 48 | 206.4265 | 0.5078 | 8.8101 | 8.5342 | 7.7268 | 20.2979 | 72 | 126.4783 | 1.6479 | 20.3106 | 18.4516 | 9.2835 | 33.9782 |
| 3-8b | 48 | 214.2092 | 0.2102 | 10.7227 | 4.2273 | 8.8819 | 16.6365 | 72 | 134.4381 | 0.5293 | 28.1733 | 8.2605 | 11.3677 | 32.4095 |
| 3-8b | 48 | 215.9521 | 0.2206 | 10.6394 | 4.2398 | 8.9096 | 16.8717 | 72 | 135.9608 | 0.4866 | 27.9994 | 8.1584 | 11.428 | 32.4438 |
| 1-29c | 48 | 202.2317 | 0.7141 | 7.1013 | 9.2615 | 8.9316 | 20.2024 | 72 | 113.6071 | 2.4498 | 19.2369 | 20.9385 | 11.0189 | 35.6876 |
| 1-29c | 48 | 204.3424 | 0.6785 | 7.0799 | 8.9315 | 9.1823 | 19.7549 | 72 | 114.7926 | 2.3853 | 18.8794 | 20.246 | 11.5163 | 34.0902 |
| 3-9a | 48 | 214.5816 | 0.117 | 8.5717 | 6.8347 | 11.881 | 14.7871 | 72 | 135.3102 | 0.2342 | 23.9701 | 12.2466 | 14.4644 | 27.9636 |
| 3-9b | 48 | 210.539 | 0.126 | 10.7309 | 4.3419 | 12.227 | 18.0502 | 72 | 137.5744 | 0.2869 | 27.547 | 7.5096 | 15.596 | 31.2168 |
| 3-9c | 48 | 214.9778 | 0.1286 | 8.9054 | 4.9065 | 8.6845 | 16.9243 | 72 | 134.3951 | 0.358 | 24.3862 | 9.8124 | 10.8007 | 32.5989 |
| 3-9d | 48 | 216.938 | 0.128 | 8.6441 | 4.8035 | 8.4098 | 16.6322 | 72 | 135.0235 | 0.3647 | 23.8622 | 9.6959 | 10.6994 | 32.4314 |
| 1-34d | 48 | 206.6119 | 0.5933 | 5.8843 | 10.0031 | 7.6589 | 20.5251 | 72 | 128.1508 | 1.7867 | 15.8991 | 21.3344 | 9.2074 | 34.3531 |
| 1-34d | 48 | 204.8085 | 0.6332 | 6.1062 | 10.3908 | 7.9665 | 20.5845 | 72 | 125.9488 | 1.9142 | 16.4945 | 22.6095 | 9.6076 | 34.0049 |
| 3-10a | 48 | 206.7055 | 0.2396 | 8.5596 | 6.674 | 8.507 | 20.5528 | 72 | 135.2835 | 0.5916 | 21.4482 | 13.2433 | 10.0429 | 33.7147 |
| 3-10b | 48 | 208.3977 | 0.2102 | 8.0937 | 6.657 | 7.9251 | 20.2586 | 72 | 134.788 | 0.459 | 21.4866 | 13.0172 | 9.2632 | 33.5134 |
| 3-10c | 48 | 204.753 | 0.2454 | 8.293 | 7.0482 | 8.4243 | 21.1464 | 72 | 122.3204 | 0.4911 | 24.3933 | 12.1151 | 10.3322 | 37.7402 |
| 3-10d | 48 | 212.2014 | 0.3232 | 7.1306 | 7.213 | 8.8215 | 18.2059 | 72 | 134.8306 | 0.9637 | 19.7932 | 16.0601 | 10.6208 | 31.7317 |
| 3-10e | 48 | 227.9907 | 0.1918 | 6.795 | 5.901 | 8.2239 | 14.423 | 72 | 153.2968 | 0.3566 | 18.7614 | 11.9417 | 10.4749 | 28.3994 |
| 2-1c | 48 | 208.1863 | 0.4397 | 8.9759 | 8.1913 | 7.9671 | 18.4494 | 72 | 125.37 | 1.6578 | 22.9459 | 18.3001 | 9.3341 | 34.2322 |
| 2-1c | 48 | 207.6098 | 0.4656 | 9.2434 | 8.331 | 7.9829 | 18.5991 | 72 | 123.4858 | 1.6909 | 23.0282 | 18.5249 | 9.3105 | 34.1316 |
| 3-11a | 48 | 220.696 | 0.1179 | 9.1501 | 4.4062 | 8.9638 | 14.5853 | 72 | 131.1657 | 0.2807 | 27.8063 | 8.8984 | 12.3057 | 31.9859 |
| 3-11b | 48 | 214.0806 | 0.1459 | 10.8701 | 5.4352 | 8.5624 | 17.3377 | 72 | 132.4178 | 0.4766 | 28.7197 | 11.597 | 10.1412 | 31.8757 |
| 3-11c | 48 | 217.6527 | 0.0868 | 10.2404 | 4.9644 | 7.9792 | 16.1662 | 72 | 136.7961 | 0.2038 | 28.2281 | 10.0485 | 9.3256 | 31.6762 |
| 3-11d | 48 | 217.5957 | 0.0938 | 10.3528 | 4.9128 | 8.1089 | 16.4785 | 72 | 138.4799 | 0.1855 | 27.4121 | 9.8514 | 9.5203 | 31.4354 |
| 3-11e | 48 | 209.5719 | 0.1015 | 10.246 | 4.7238 | 9.5375 | 18.013 | 72 | 129.4291 | 0.2604 | 28.3948 | 8.8626 | 11.9304 | 32.6698 |
| 2-2b | 48 | 199.6935 | 2.5145 | 8.4779 | 10.5693 | 9.3283 | 19.0321 | 72 | 108.7979 | 8.382 | 21.4922 | 22.5305 | 10.931 | 34.9665 |
| 2-2b | 48 | 201.5703 | 2.4835 | 8.4864 | 10.0494 | 9.4722 | 18.8787 | 72 | 108.8986 | 8.4164 | 21.0962 | 21.8325 | 11.1892 | 34.5578 |
| 3-12a | 48 | 213.4043 | 0.2864 | 10.4215 | 4.5361 | 9.705 | 17.5497 | 72 | 136.7883 | 0.7776 | 26.4606 | 9.5696 | 10.9074 | 32.0088 |
| 3-12b | 48 | 214.0301 | 0.5089 | 9.2185 | 4.2507 | 11.3005 | 14.8197 | 72 | 132.8362 | 1.3071 | 27.58 | 8.7051 | 14.5642 | 30.6476 |
| 3-12c | 48 | 218.9281 | 0.2914 | 11.0618 | 4.9739 | 9.5165 | 14.4665 | 72 | 143.4417 | 0.6795 | 28.3727 | 9.645 | 11.7592 | 28.5572 |
| 3-12d | 48 | 207.0825 | 0.2581 | 13.6285 | 4.8446 | 10.8487 | 17.7572 | 72 | 117.46 | 0.6934 | 34.3581 | 10.2581 | 12.7358 | 34.7806 |
| 3-12e | 48 | 215.7847 | 0.4839 | 8.8245 | 4.2513 | 11.8297 | 14.262 | 72 | 131.5246 | 1.3085 | 27.5444 | 8.689 | 15 | 30.5545 |
| 1-30b | 48 | 200.0981 | 0.8939 | 6.2791 | 10.8533 | 9.1344 | 22.4863 | 72 | 107.4816 | 2.6963 | 18.3466 | 22.7463 | 11.1086 | 37.9321 |
| 1-30b | 48 | 196.8494 | 0.8847 | 6.0131 | 10.7051 | 8.7571 | 22.4401 | 72 | 110.2649 | 2.6078 | 18.0476 | 22.0978 | 10.6952 | 38.7419 |
| 3-13a | 48 | 201.5396 | 0.1206 | 6.8686 | 6.9277 | 9.8557 | 21.8287 | 72 | 121.7689 | 0.263 | 20.9284 | 13.4668 | 12.1844 | 37.4305 |
| 3-13b | 48 | 202.9155 | 0.1886 | 9.1697 | 5.7259 | 9.4161 | 21.8607 | 72 | 121.2763 | 0.4347 | 26.0852 | 10.6865 | 11.7444 | 37.4292 |
| 3-13c | 48 | 208.3318 | 0.1487 | 7.0506 | 6.2369 | 11.6856 | 18.7354 | 72 | 120.4372 | 0.3535 | 22.7982 | 11.9999 | 14.4255 | 35.8198 |
| 3-13d | 48 | 186.8012 | 0.7748 | 5.5433 | 11.7699 | 11.4631 | 24.719 | 72 | 102.1726 | 2.1821 | 16.5656 | 22.7494 | 13.6967 | 39.3186 |
| 3-13e | 48 | 210.2575 | 0.2991 | 7.4933 | 6.4249 | 9.2085 | 19.439 | 72 | 123.1344 | 0.8901 | 22.5794 | 13.1108 | 11.2205 | 36.3659 |
| 1-13c | 48 | 210.4435 | 0.5718 | 8.3661 | 8.0227 | 7.7207 | 19.1573 | 72 | 113.2521 | 2.0392 | 21.2114 | 19.686 | 9.5037 | 35.9956 |
| 1-13c | 48 | 210.9215 | 0.5985 | 8.5164 | 8.0827 | 7.4482 | 20.3852 | 72 | 117.7919 | 2.0306 | 21.445 | 19.3047 | 9.041 | 37.2486 |
| 1-31d | 48 | 201.0846 | 0.4715 | 4.9787 | 12.9489 | 8.0189 | 23.2925 | 72 | 112.3435 | 1.1537 | 14.429 | 28.1226 | 9.5516 | 38.6495 |
| 1-31d | 48 | 198.3987 | 0.4864 | 5.0698 | 13.1211 | 8.1166 | 23.6602 | 72 | 111.4464 | 1.134 | 14.6546 | 27.721 | 9.5524 | 39.754 |
| 3-14a | 48 | 213.3737 | 0.1892 | 5.8535 | 7.8386 | 9.0585 | 19.9807 | 72 | 134.9562 | 0.3535 | 17.4087 | 15.5685 | 10.9231 | 34.2413 |
| 3-14b | 48 | 215.4885 | 0.153 | 5.7627 | 7.4993 | 7.5363 | 19.9338 | 72 | 127.2369 | 0.2659 | 18.2595 | 15.5165 | 9.1061 | 37.8411 |
| 3-14c | 48 | 199.5026 | 0.1896 | 8.7171 | 6.8805 | 8.7709 | 23.7636 | 72 | 117.4626 | 0.3399 | 23.6883 | 14.4865 | 10.8493 | 39.0769 |
| 3-14d | 48 | 203.4428 | 0.161 | 7.0024 | 7.5194 | 9.1469 | 23.0752 | 72 | 123.1288 | 0.2697 | 20.2639 | 15.153 | 11.1169 | 37.4474 |
| 3-14e | 48 | 204.0004 | 0.1554 | 6.949 | 7.8763 | 8.7589 | 22.8729 | 72 | 120.7003 | 0.2839 | 20.8338 | 16.1304 | 10.9268 | 38.489 |
| 1-32d | 48 | 213.3481 | 0.5836 | 8.1706 | 7.3433 | 6.8721 | 20.7383 | 72 | 123.5824 | 1.7676 | 22.0714 | 17.265 | 8.5331 | 38.2365 |
| 1-32d | 48 | 210.4628 | 0.6055 | 7.9386 | 8.564 | 9 | 18.9474 | 72 | 108.6632 | 2.0998 | 23.7377 | 21.3464 | 11.2798 | 34.145 |
| 3-15a | 48 | 234.7892 | 0.1303 | 9.1585 | 4.1524 | 6.9298 | 13.0591 | 72 | 136.9647 | 0.2062 | 27.3678 | 9.3599 | 9.8196 | 34.0258 |
| 3-15b | 48 | 223.5736 | 0.2266 | 8.3303 | 4.6764 | 7.2986 | 17.5254 | 72 | 138.6352 | 0.6421 | 24.199 | 10.7286 | 9.0755 | 34.944 |
| 3-15c | 48 | 224.0437 | 0.1312 | 9.3328 | 4.0255 | 6.842 | 17.8585 | 72 | 139.6446 | 0.2711 | 26.2045 | 8.8188 | 8.2638 | 34.6002 |
| 3-15d | 48 | 226.0387 | 0.0913 | 9.4441 | 4.2311 | 7.6411 | 15.7256 | 72 | 139.6553 | 0.1469 | 26.2966 | 8.7961 | 9.3674 | 33.9378 |
| 3-15e | 48 | 221.0996 | 0.1795 | 9.6165 | 4.5582 | 7.7267 | 17.5275 | 72 | 128.8977 | 0.4779 | 27.5285 | 10.6097 | 9.5609 | 34.8858 |
| 1-14d | 48 | 201.3263 | 14.0487 | 3.7314 | 5.5296 | 6.405 | 21.6154 | 72 | 104.3875 | 34.7256 | 12.4323 | 12.2728 | 6.9542 | 38.2912 |
| 1-14d | 48 | 201.5155 | 14.3708 | 3.7343 | 5.4153 | 6.0218 | 22.016 | 72 | 106.5205 | 34.1991 | 12.2278 | 11.953 | 6.5285 | 38.2475 |
| 3-16a | 48 | 202.5449 | 15.4237 | 2.7986 | 5.5488 | 6.2779 | 21.5848 | 72 | 94.5105 | 42.0578 | 8.9267 | 13.0326 | 6.6157 | 41.2429 |
| 3-16b | 48 | 190.2844 | 17.886 | 3.1826 | 6.4714 | 7.336 | 23.7065 | 72 | 72.576 | 48.6039 | 9.6915 | 14.3203 | 7.8497 | 45.5983 |
| 3-16c | 48 | 212.9587 | 15.095 | 2.4801 | 5.3867 | 4.6951 | 18.8608 | 72 | 91.3416 | 44.5217 | 8.2484 | 14.1067 | 5.5264 | 41.8397 |
| 3-16d | 48 | 197.5323 | 15.9026 | 3.0689 | 5.7751 | 6.4189 | 23.067 | 72 | 73.78 | 45.7646 | 10.1304 | 13.4115 | 7.8042 | 46.7786 |
| 3-16e | 48 | 198.4138 | 16.6462 | 3.0247 | 5.8989 | 6.6123 | 22.9064 | 72 | 87.814 | 44.9453 | 9.2729 | 13.5817 | 6.9859 | 41.4791 |
| 1-16a | 48 | 223.5495 | 0.778 | 5.8152 | 8.2518 | 7.1368 | 15.9472 | 72 | 132.0333 | 2.7971 | 17.6658 | 21.216 | 9.2676 | 33.375 |
| 1-16a | 48 | 222.7868 | 0.7005 | 6.1369 | 8.4551 | 7.1298 | 15.9604 | 72 | 130.3811 | 2.6259 | 18.0057 | 21.6928 | 9.3127 | 33.1296 |
| 3-17a | 48 | 235.2271 | 0.2358 | 6.6069 | 5.4046 | 8.5695 | 12.4699 | 72 | 153.4769 | 0.7268 | 20.491 | 11.9957 | 10.5556 | 28.7039 |
| 3-17b | 48 | 230.3205 | 0.1993 | 7.715 | 6.3352 | 8.7712 | 13.1744 | 72 | 151.2753 | 0.3924 | 24.1088 | 12.598 | 10.9009 | 26.0664 |
| 3-17c | 48 | 227.1336 | 0.1776 | 8.1043 | 5.0795 | 8.0004 | 14.8424 | 72 | 145.2595 | 0.3122 | 25.504 | 10.5849 | 10.1598 | 29.4796 |
| 3-17d | 48 | 228.0835 | 0.1851 | 7.8429 | 5.8428 | 8.2445 | 14.2311 | 72 | 147.9796 | 0.3232 | 23.7175 | 11.2347 | 10.2623 | 29.8074 |
| 3-17e | 48 | 230.3581 | 0.1539 | 7.5679 | 4.8848 | 7.8693 | 13.8068 | 72 | 155.9219 | 0.2679 | 23.2781 | 9.9935 | 10.0803 | 28.107 |
| 1-13c | 96 | 63.4521 | 2.7799 | 37.0407 | 24.425 | 10.937 | 39.1855 | 120 | 57.921 | 3.111 | 40.358 | 24.781 | 11.453 | 31.801 |
| 1-13c | 96 | 63.0603 | 2.8335 | 37.2163 | 24.8975 | 10.904 | 39.0075 | 120 | 57.503 | 3.178 | 40.693 | 25.447 | 11.504 | 34.413 |
| 3-8b | 96 | 76.9539 | 1.0617 | 48.6449 | 10.6604 | 14.441 | 36.7838 | 120 | 69.872 | 1.305 | 53.305 | 11.107 | 15.485 | 32.899 |
| 3-8b | 96 | 77.8085 | 1.0223 | 48.6021 | 10.588 | 14.3952 | 36.8228 | 120 | 69.142 | 1.267 | 53.48 | 10.997 | 15.503 | 32.688 |
| 1-29c | 96 | 42.6468 | 4.0335 | 36.9756 | 28.8612 | 12.7255 | 42.9411 | 120 | 27.645 | 4.51 | 42.125 | 30.002 | 13.397 | 39.176 |
| 1-29c | 96 | 42.6026 | 4.1622 | 36.8468 | 29.4261 | 13.6701 | 39.3934 | 120 | 21.972 | 4.767 | 43.744 | 31.549 | 14.657 | 32.154 |
| 3-9a | 96 | 66.1277 | 0.5032 | 47.9634 | 18.5994 | 17.0186 | 35.375 | 120 | 18.14 | 0.667 | 65.083 | 20.549 | 18.744 | 37.274 |
| 3-9b | 96 | 77.0519 | 0.449 | 46.5399 | 9.5868 | 19.404 | 38.0281 | 120 | 71.819 | 0.531 | 50.263 | 9.818 | 20.352 | 33.602 |
| 3-9c | 96 | 66.4487 | 0.6508 | 45.339 | 13.7378 | 12.6052 | 40.6981 | 120 | 47.029 | 0.86 | 52.928 | 14.558 | 13.235 | 38.494 |
| 3-9d | 96 | 66.4471 | 0.6285 | 44.5028 | 13.5423 | 12.6663 | 41.0265 | 120 | 40.227 | 0.849 | 53.797 | 14.069 | 13.483 | 41.214 |
| 1-34d | 96 | 64.5126 | 2.9967 | 30.1 | 29.0299 | 10.9305 | 40.2026 | 120 | 54.959 | 3.404 | 33.849 | 29.945 | 11.469 | 36.154 |
| 1-34d | 96 | 61.2008 | 3.2157 | 30.6236 | 30.7488 | 11.415 | 39.6719 | 120 | 51.589 | 3.59 | 34.48 | 32.042 | 11.96 | 34.745 |
| 3-10a | 96 | 76.038 | 1.0011 | 38.5657 | 17.5169 | 11.8706 | 38.7155 | 120 | 68.776 | 1.155 | 42.549 | 17.945 | 12.475 | 35.343 |
| 3-10b | 96 | 74.3722 | 0.7129 | 39.1155 | 17.4278 | 11.1215 | 39.2745 | 120 | 62.964 | 0.868 | 44.239 | 17.932 | 11.886 | 35.557 |
| 3-10c | 96 | 76.7161 | 0.7192 | 37.3364 | 13.3757 | 11.6396 | 40.7541 | 120 | 78.057 | 0.814 | 39.569 | 13.358 | 12.086 | 34.93 |
| 3-10d | 96 | 70.2632 | 1.6937 | 36.6955 | 23.4892 | 12.5872 | 37.939 | 120 | 48.012 | 2.072 | 43.954 | 24.744 | 13.608 | 35.654 |
| 3-10e | 96 | 89.9386 | 0.5933 | 36.4406 | 17.7283 | 12.5889 | 35.1673 | 120 | 61.842 | 0.799 | 46.294 | 18.624 | 14.217 | 36.035 |
| 2-1c | 96 | 62.7342 | 2.4718 | 38.3082 | 22.2746 | 11.6038 | 39.6956 | 120 | 64.76 | 2.64 | 40.477 | 22.511 | 11.928 | 33.049 |
| 2-1c | 96 | 63.2282 | 2.455 | 38.1549 | 22.4225 | 11.4255 | 40.3061 | 120 | 64.921 | 2.661 | 39.844 | 22.361 | 11.729 | 34.435 |
| 3-11a | 96 | 79.7936 | 0.436 | 39.203 | 9.5057 | 13.9519 | 39.3012 | 120 | 82.43 | 0.488 | 41.473 | 9.61 | 14.443 | 33.445 |
| 3-11b | 96 | 64.3457 | 0.7527 | 48.4241 | 14.6685 | 12.2579 | 38.1891 | 120 | 62.566 | 0.845 | 52.008 | 15.063 | 12.835 | 31.72 |
| 3-11c | 96 | 72.6833 | 0.3207 | 47.5852 | 12.5245 | 11.854 | 36.2887 | 120 | 70.217 | 0.383 | 51.399 | 12.741 | 12.618 | 29.347 |
| 3-11d | 96 | 72.4651 | 0.304 | 46.2975 | 12.2904 | 11.7992 | 38.4054 | 120 | 71.613 | 0.378 | 49.607 | 12.463 | 12.344 | 32.168 |
| 3-11e | 96 | 74.6271 | 0.4053 | 44.372 | 10.9302 | 14.687 | 37.7466 | 120 | 74.576 | 0.444 | 47.025 | 11.008 | 15.177 | 33.28 |
| 2-2b | 96 | 39.7812 | 13.7664 | 38.7171 | 28.8975 | 12.8476 | 40.9686 | 120 | 35.288 | 15.25 | 41.686 | 29.644 | 13.366 | 37.038 |
| 2-2b | 96 | 36.9018 | 14.4368 | 38.7983 | 29.095 | 13.1847 | 39.3229 | 120 | 25.136 | 16.677 | 43.693 | 30.669 | 13.947 | 32.382 |
| 3-12a | 96 | 88.7973 | 1.344 | 42.8443 | 11.4926 | 12.1264 | 32.5453 | 120 | 87.455 | 1.668 | 46.968 | 11.846 | 12.8 | 27.581 |
| 3-12b | 96 | 75.3895 | 2.3564 | 46.6193 | 11.8914 | 16.3296 | 36.0919 | 120 | 44.319 | 3.504 | 56.318 | 11.731 | 17.529 | 37.34 |
| 3-12c | 96 | 79.4903 | 1.3014 | 50.5823 | 13.9091 | 13.5625 | 34.4906 | 120 | 37.289 | 2.188 | 69.731 | 16.082 | 15.985 | 35.638 |
| 3-12d | 96 | 62.5057 | 1.3402 | 54.4955 | 12.966 | 14.8321 | 37.9394 | 120 | 60.515 | 1.52 | 58.168 | 12.981 | 15.365 | 33.222 |
| 3-12e | 96 | 73.1334 | 2.3688 | 46.2983 | 11.7394 | 16.6601 | 36.2572 | 120 | 43.482 | 3.63 | 56.535 | 11.854 | 18.003 | 37.693 |
| 1-30b | 96 | 52.221 | 3.6826 | 30.127 | 26.0419 | 12.204 | 41.6801 | 120 | 48.892 | 4.014 | 32.7 | 26.54 | 12.772 | 33.989 |
| 1-30b | 96 | 55.7781 | 3.5616 | 29.3514 | 25.1048 | 11.8132 | 44.309 | 120 | 53.32 | 3.863 | 31.673 | 25.533 | 12.233 | 38.787 |
| 3-13a | 96 | 69.6485 | 0.3884 | 32.082 | 15.5544 | 13.62 | 42.4193 | 120 | 67.036 | 0.432 | 34.231 | 15.425 | 13.991 | 38.003 |
| 3-13b | 96 | 60.9447 | 0.7128 | 41.964 | 12.3895 | 13.1632 | 45.1307 | 120 | 53.66 | 0.79 | 45.98 | 12.696 | 13.806 | 41.696 |
| 3-13c | 96 | 67.2225 | 0.5871 | 34.9839 | 13.3457 | 15.6583 | 41.2066 | 120 | 62.029 | 0.655 | 38.436 | 13.456 | 16.355 | 36.093 |
| 3-13d | 96 | 63.9351 | 2.7768 | 24.2476 | 24.6932 | 14.9839 | 41.4449 | 120 | 64.806 | 2.991 | 26.075 | 25.474 | 15.547 | 35.862 |
| 3-13e | 96 | 67.073 | 1.3394 | 36.1148 | 15.3901 | 12.3532 | 42.8279 | 120 | 60.562 | 1.486 | 39.654 | 15.629 | 12.918 | 38.898 |
| 1-13c | 96 | 42.7416 | 3.4145 | 39.8105 | 27.0726 | 11.0804 | 41.5409 | 120 | 29.5691 | 3.442 | 44.3005 | 27.678 | 11.6517 | 34.9042 |
| 1-13c | 96 | 47.7423 | 3.3648 | 39.5227 | 26.4578 | 10.5224 | 41.8028 | 120 | 34.6813 | 3.418 | 44.5072 | 27.3524 | 11.2435 | 35.5544 |
| 1-31d | 96 | 50.4882 | 1.7156 | 25.5501 | 36.5828 | 11.4828 | 43.4847 | 120 | 45.7733 | 1.7249 | 27.4232 | 37.4522 | 12.1452 | 36.2939 |
| 1-31d | 96 | 52.4217 | 1.641 | 25.443 | 34.9485 | 11.4024 | 45.2072 | 120 | 49.3992 | 1.6179 | 26.6209 | 35.3383 | 11.8009 | 38.7378 |
| 3-14a | 96 | 80.7118 | 0.4906 | 29.8098 | 20.5898 | 12.7561 | 38.6428 | 120 | 73.4301 | 0.5156 | 32.2873 | 20.894 | 13.4017 | 33.6381 |
| 3-14b | 96 | 58.2841 | 0.4136 | 34.1657 | 20.6833 | 11.5352 | 41.9877 | 120 | 52.3897 | 0.4679 | 37.0043 | 21.3136 | 12.3005 | 31.7803 |
| 3-14c | 96 | 55.2876 | 0.4807 | 40.2838 | 18.3933 | 13.1057 | 44.0425 | 120 | 42.0233 | 0.5263 | 43.9259 | 18.6769 | 13.7704 | 38.9515 |
| 3-14d | 96 | 66.1412 | 0.3724 | 33.1151 | 18.7354 | 13.1573 | 40.9171 | 120 | 60.5665 | 0.4136 | 35.1723 | 19.2314 | 13.8416 | 33.6682 |
| 3-14e | 96 | 63.8914 | 0.3774 | 32.7028 | 19.2733 | 12.8829 | 42.318 | 120 | 60.5607 | 0.4093 | 34.2322 | 19.7058 | 13.5662 | 36.2568 |
| 1-32d | 96 | 50.2745 | 2.6069 | 40.2013 | 22.7031 | 10.042 | 45.0593 | 120 | 46.3015 | 2.566 | 42.3881 | 22.9682 | 10.4076 | 38.7555 |
| 1-32d | 96 | 34.3182 | 3.32 | 42.5684 | 30.7898 | 13.0702 | 38.2206 | 120 | 30.3532 | 3.3178 | 46.3197 | 32.2766 | 13.906 | 25.9821 |
| 3-15a | 96 | 62.0032 | 0.3217 | 48.4635 | 12.6197 | 11.8388 | 42.968 | 120 | 59.9164 | 0.276 | 51.763 | 12.5845 | 12.3462 | 31.1046 |
| 3-15b | 96 | 61.9058 | 1.0493 | 45.6853 | 15.7093 | 10.817 | 42.3545 | 120 | 43.0642 | 1.0177 | 52.213 | 16.1092 | 11.4262 | 38.5257 |
| 3-15c | 96 | 65.6874 | 0.4161 | 48.0933 | 12.5032 | 9.9044 | 43.122 | 120 | 49.5255 | 0.4536 | 54.2507 | 12.5874 | 10.4089 | 38.5121 |
| 3-15d | 96 | 59.5905 | 0.1882 | 49.3534 | 12.1457 | 10.8522 | 44.6433 | 120 | 37.5445 | 0.2323 | 57.001 | 12.2024 | 11.5288 | 40.6026 |
| 3-15e | 96 | 49.2206 | 0.7603 | 50.406 | 15.4751 | 11.3633 | 41.5041 | 120 | 34.8627 | 0.8584 | 57.0693 | 15.7986 | 12.0298 | 32.7456 |
| 1-14d | 96 | 19.9788 | 61.977 | 19.112 | 14.6612 | 7.3285 | 46.2224 | 120 | 16.3562 | 64.136 | 20.6813 | 14.952 | 7.5863 | 38.1461 |
| 1-14d | 96 | 23.5118 | 60.0102 | 18.7427 | 14.3505 | 6.9811 | 46.8423 | 120 | 18.7054 | 62.0462 | 20.1118 | 14.4347 | 7.1773 | 39.9858 |
| 3-16a | 96 | 17.8194 | 68.6178 | 13.2008 | 14.6202 | 6.8959 | 48.1906 | 120 | 17.9355 | 68.6861 | 14.2279 | 14.8443 | 7.0357 | 40.9093 |
| 3-16b | 96 | 9.3898 | 71.1379 | 13.1275 | 15.0732 | 8.1035 | 48.9135 | 120 | 9.9534 | 71.4309 | 14.2217 | 15.3554 | 8.2618 | 41.2089 |
| 3-16c | 96 | 18.5623 | 69.0885 | 12.039 | 15.4589 | 5.7518 | 46.7611 | 120 | 19.0039 | 69.8587 | 12.6134 | 15.7055 | 5.9539 | 36.1429 |
| 3-16d | 96 | 22.9723 | 62.6835 | 12.7223 | 14.4051 | 8.3691 | 47.3743 | 120 | 23.6036 | 62.9397 | 13.6683 | 14.6057 | 8.5864 | 38.1746 |
| 3-16e | 96 | 15.5398 | 70.9855 | 13.532 | 15.1719 | 7.2816 | 46.734 | 120 | 15.5755 | 70.924 | 14.6609 | 15.3809 | 7.4565 | 37.967 |
| 1-16a | 96 | 54.8639 | 4.2448 | 34.3506 | 31.6253 | 10.7044 | 42.287 | 120 | 19.1881 | 4.3593 | 44.0697 | 32.4773 | 11.4123 | 42.3068 |
| 1-16a | 96 | 52.8323 | 4.0332 | 34.4472 | 32.4192 | 10.7579 | 42.1739 | 120 | 16.1096 | 4.2806 | 45.1314 | 33.9014 | 11.6628 | 42.2204 |
| 3-17a | 96 | 72.5281 | 1.287 | 40.8205 | 20.3579 | 12.3021 | 35.9767 | 120 | 20.225 | 1.4858 | 58.264 | 23.3105 | 13.808 | 35.0088 |
| 3-17b | 96 | 81.7669 | 0.5664 | 44.8808 | 19.2761 | 12.0288 | 32.983 | 120 | 27.9248 | 0.6709 | 65.5745 | 23.738 | 13.5351 | 33.0027 |
| 3-17c | 96 | 72.3766 | 0.4173 | 47.0391 | 15.9791 | 11.6034 | 36.7485 | 120 | 28.3323 | 0.5141 | 61.964 | 16.9871 | 12.702 | 38.293 |
| 3-17d | 96 | 79.9688 | 0.4264 | 44.8671 | 16.7224 | 11.2857 | 37.182 | 120 | 29.3626 | 0.5166 | 63.3376 | 19.0267 | 12.6103 | 39.4502 |
| 3-17e | 96 | 79.5091 | 0.3497 | 43.7383 | 15.2142 | 11.5541 | 37.9609 | 120 | 32.4141 | 0.4243 | 58.9057 | 15.9593 | 12.7063 | 41.3602 |

### Example 16: Phosphatase Diversity

The final step in the xylitol pathway, from xylitol 5-phosphate to xylitol, requires a phosphatase enzyme. The Saccharomyces cerevisiae PYP1 (polyol phosphatase 1) gene encodes a sugar alcohol phosphatase that hydrolyzes sorbitol-6-phosphate, ribitol-5-phosphate, and (D)-glycerol-3-phosphase (Xu et al., “Discovery and functional characterization of a yeast sugar alcohol phosphatase,” ACS Chem. Biol., 13, 2018, 3011-3020). PYP1 is a member of the haloacid dehalogenase (HAD)-like hydrolase superfamily (Kuznetsova, et a., “Functional diversity of haloacid dehalogenase superfamily phosphatases from Saccharomyces cerevisiae,” J. Biol. Chem., 2015, 290, 18678-18698) and belongs to the enzyme class of sorbitol-6-phosphatases (EC 3.1.3.50). As xylitol 5-phosphate is a similar molecule to the known substrates of PYP1, an ortholog of PYP1 in Moniliella is likely responsible for the final catalytic step in the pathway for xylitol production. A query of the Moniliella pollinis genome of strain 1-1 found two genes with high homology to PYP1; RCSR00371 and RCSR15215. These two genes are 40% and 38% identical to ScPYP1, respectively, and 89% identical to each other.

E. coli HxpA (hexitol phosphatase A) is a HAD-like enzyme belonging to EC 3.1.3.50 with a similar substrate profile to PYP1 (Kuznetsova et al., “Genome wide analysis of substrate specificities of the Escherichia coli haloacid dehalogenase-like phosphate family,” 2006, J. Biol. Chem., 281, 36149-36161). HxpA and PYP1 share low sequence similarity, and therefore the M. pollinis genome was searched for HxpA orthologs as alternative phosphatase candidates. RCSR21016 was identified as 38% identical to HxpA and independently identified as a sugar phosphatase with sequence similarity to S. cerevisiae DOG1 and DOG2. As shown in Examples 12-15, S. cerevisiae DOG1 and DOG2 expression increases xylitol production in M. pollinis.

Based on enzyme classification and/or sequence identity to the M. pollinis RCSR00371, RCSR15215, and RCSR21016 phosphatases and the S. cerevisiae PYP1 phosphatase, 26 additional phosphatase candidates were chosen for further analysis, as outlined in Table 19.

TABLE 19
Phosphatase

| | | Polypeptide |
|---|---|---|
| Source Organism | Gene Name/Accession Numbers | SEQ ID NO: |
| Moniliella pollinis | RCSR00371 | 198 |
| Moniliella pollinis | RCSR15215 | 199 |
| Moniliella pollinis | RCSR21016 | 200 |
| Saccharomyces cerevisiae | PYP1 | 201 |
| Lachancea dasiensis | HAD; GenBank SCU98827.1 | 202 |
| Tetrapisispora blattae | HAD; GenBank XP_004180453.1 | 203 |
| Saccharomyces pastorianus | HAD; GenBank QID87760.1 | 204 |
| Kazachstania africana | HAD; GenBank XP_003958862.1 | 205 |
| Podospora comata | HAD; GenBank VBB84572.1 | 206 |
| Geotrichum candidum | HAD; GenBank KAF5104503.1 | 207 |
| Ogattaea haglerorum | HAD; GenBank KAG7698539.1 | 208 |
| Debaryomyces fabryi | HAD; GenBank XP_015467979.1 | 209 |
| Monilinia fructicola | HAD; GenBank KAG4025226.1 | 210 |
| Nadsonia fulvescens | HAD; GenBank ODQ66340.1 | 211 |
| var. elongata DSM 6958 | | |
| Actinomadura macrotermitis | HAD; GenBank WP_153530600.1 | 212 |
| Escherichia coli | HxpA; GenBank WP_001203392.1 | 213 |
| Wickerhamomyces ciferrii | HAD; GenBank XP_011272915.1 | 214 |
| Candida maltosa | 4NPP; GenBank EMG50545.1 | 215 |
| Paenibacillus | HAD; GenBank WP_095357324.1 | 216 |
| Escherichia coli | HAD; GenBank EEC8032393.1 | 217 |
| Mycolicibacterium smegmatis | ; GenBank WP_011727842.1 | 218 |
| Amycolatopsis sp. CA-230715 | HAD; GenBank WP_254125895.1 | 219 |
| Streptomyces smaragdinus | ; GenBank MQY10897.1 | 220 |
| Moniliella pollinis | PYP1-3 | 221 |
| Bacillus amyloliquefaciens | HAD; GenBank WP_115997730.1 | 222 |

### Example 17: Genetically Modified Moniliella pollinis Strains

The indicated Moniliella pollinis parent strain was transformed with the bipartite transformation fragments as indicated in Table 20 using the transformation method outlined in Example 4. Resulting transformants were evaluated by colony PCR for integration of the indicated sequence. A PCR verified isolate was then designated as the indicated strain number in Table 20. In some instances, more than one PCR verified isolate, e.g., “sister” isolates, are indicated by letters following the strain number. For example, strain 4-1 has 5 sister isolates, strains 4-1a, 4-1b, 4-1c, 4-1d, and 4-1e.

For example, Strain 2-2b was transformed with SEQ ID NO:223 and SEQ ID NO: 181. SEQ ID NO:181 is described in Example 12 and contains, in order, a 5′ ER3 flanking sequence (SEQ ID NO:155), an MpPYK1 promoter (SEQ ID NO:86), a gene encoding the M. pollinis RPE2 polypeptide of SEQ ID NO:180, an MpPYK1 terminator (SEQ ID NO:146), and a 5′ portion of a zeocin resistance gene expression cassette (SEQ ID NO:169). SEQ ID NO:223 contains a 3′ portion of a zeocin resistance gene expression cassette (SEQ ID NO:168), an MpPGK1 promoter (SEQ ID NO:135), a gene (SEQ ID NO:224) encoding the S. cerevisiae PYP1 polypeptide of SEQ ID NO: 201, an Mp6PGD terminator (SEQ ID NO:140), and a 3′ ER3 flanking sequence (SEQ ID NO: 165). Transformants were selected on PDA+zeocin selection plates and incubated at 35 °C for at least 2 days until transformants grow. Resulting transformants were streaked for single colony isolation on PDA+zeocin plates and single colonies were selected. Selected colonies were evaluated by colony PCR for integration of the indicated sequence. PCR verified isolates were designated strains 4-1a, 4-1b, 4-1c, 4-1d, and 4-1e.

Transformation cassettes of the phosphatase homologs outlined in Table 20 have the same components as SEQ ID NO:223 above but included the indicated nucleotide sequence encoding the indicated polypeptide sequence.

TABLE 20

| | | Phosphatase Homolog Expression Cassette | | | Second Bipartite Cassette | |
|---|---|---|---|---|---|---|
| | | | | Encoded | | Encoded |
| | Parent | Transformation | Nucleotide | Polypeptide | Transformation | Polypeptide |
| Strain | Strain | SEQ ID NO: | SEQ ID NO: | SEQ ID NO: | SEQ ID NO: | SEQ ID NO: |
| 4-1a-e | 2-2b | 223 | 224 | 201 | 181 | 180 |
| 4-2a-e | 2-2b | 225 | 226 | 198 | 181 | 180 |
| 4-3a-e | 2-2b | 227 | 228 | 199 | 181 | 180 |
| 4-4a-e | 2-2b | 229 | 230 | 200 | 181 | 180 |
| 4-5a-e | 1-14d | 223 | 224 | 201 | 181 | 180 |
| 4-6a-b | 1-14d | 225 | 226 | 198 | 181 | 180 |
| 4-7a-e | 1-14d | 227 | 228 | 199 | 181 | 180 |
| 4-8a-e | 1-14d | 229 | 230 | 200 | 181 | 180 |
| 5-1a-e | 1-1 | 223 | 224 | 201 | 185 | na |
| 5-2a-e | 1-1 | 225 | 226 | 198 | 185 | na |
| 5-3a-e | 1-1 | 227 | 228 | 199 | 185 | na |
| 5-4a-e | 1-1 | 229 | 230 | 200 | 185 | na |
| 5-5a-e | 2-2b | 223 | 224 | 201 | 185 | na |
| 5-6a-d | 2-2b | 229 | 230 | 200 | 185 | na |
| 5-7a-e | 1-13c | 229 | 230 | 200 | 181 | 180 |
| 5-8a-e | 1-15a | 229 | 230 | 200 | 181 | 180 |
| 5-9a-e | 1-29c | 229 | 230 | 200 | 181 | 180 |
| 5-10a-e | 1-32d | 229 | 230 | 200 | 181 | 180 |
| 5-11a-e | 1-16a | 229 | 230 | 200 | 181 | 180 |
| 5-12a-e | 2-2b | 231 | 232 | 202 | 181 | 180 |
| 5-13a-d | 2-2b | 233 | 234 | 201 | 181 | 180 |
| 5-14a-e | 2-2b | 235 | 236 | 203 | 181 | 180 |
| 5-15a-e | 2-2b | 237 | 238 | 204 | 181 | 180 |
| 5-16a-e | 2-2b | 239 | 240 | 205 | 181 | 180 |
| 5-17a-e | 2-2b | 241 | 242 | 206 | 181 | 180 |
| 5-18a-e | 2-2b | 243 | 244 | 207 | 181 | 180 |
| 5-19a-d | 2-2b | 245 | 246 | 208 | 181 | 180 |
| 5-20a-d | 2-2b | 247 | 248 | 209 | 181 | 180 |
| 5-21a-c | 2-2b | 249 | 250 | 210 | 181 | 180 |
| 5-22 | | | | | | |
| 5-23a-c | 2-2b | 251 | 252 | 211 | 181 | 180 |
| 5-24a-b | | | | | | |
| 5-25a-e | 2-2b | 253 | 254 | 212 | 181 | 180 |
| 5-26a-d | 2-2b | 255 | 256 | 213 | 181 | 180 |
| 5-27 | | | | | | |
| 5-28a-d | 2-2b | 257 | 258 | 214 | 181 | 180 |
| 5-29 | | | | | | |
| 5-30a-b | 2-2b | 259 | 260 | 189 | 181 | 180 |
| 5-31a-b | | | | | | |
| 5-32a-c | 2-2b | 261 | 262 | 215 | 181 | 180 |
| 5-33 | | | | | | |
| 5-34a-c | 2-2b | 263 | 264 | 216 | 181 | 180 |
| 5-35a-c | | | | | | |
| 5-36a-b | 2-2b | 265 | 266 | 217 | 181 | 180 |
| 5-37a-c | | | | | | |
| 5-38 | 2-2b | 267 | 268 | 218 | 181 | 180 |
| 5-39a-b | 2-2b | 269 | 270 | 219 | 181 | 180 |
| 5-40a-c | | | | | | |
| 5-41a-b | 2-2b | | | | | |
| 5-42a-d | | 271 | 272 | 220 | 181 | 180 |
| 5-43a-f | 2-2b | 273 | 274 | 221 | 181 | 180 |
| 5-44a-b | 2-2b | 275 | 276 | 222 | 181 | 180 |
| 5-45a-d | | | | | | |
| 5-46 | 2-2b | 277 | 278 | 188 | 181 | 180 |
| 5-47a-e | | | | | | |

na—not applicable, second cassette used for recombination and selection marker only

### Example 18: Shake Flask Fermentation Assay

Strains 2-2b, 1-14d, 3-12c, 3-12d, 4-1a-e, 4-2a-e, 4-3a-e, 4-4a-e, 4-5a-e, 4-6a-b, 4-7a-e, and 4-8a-e were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

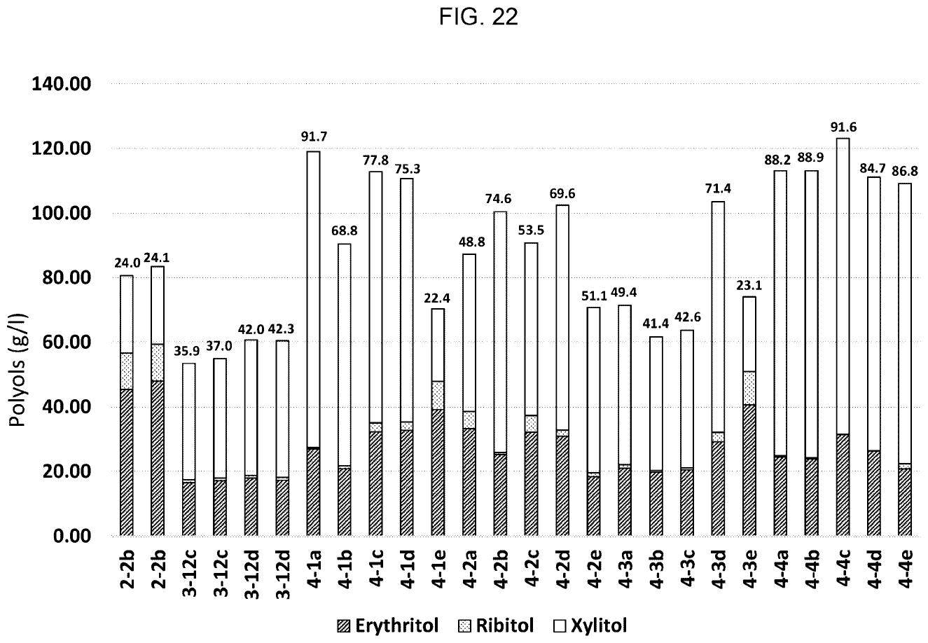

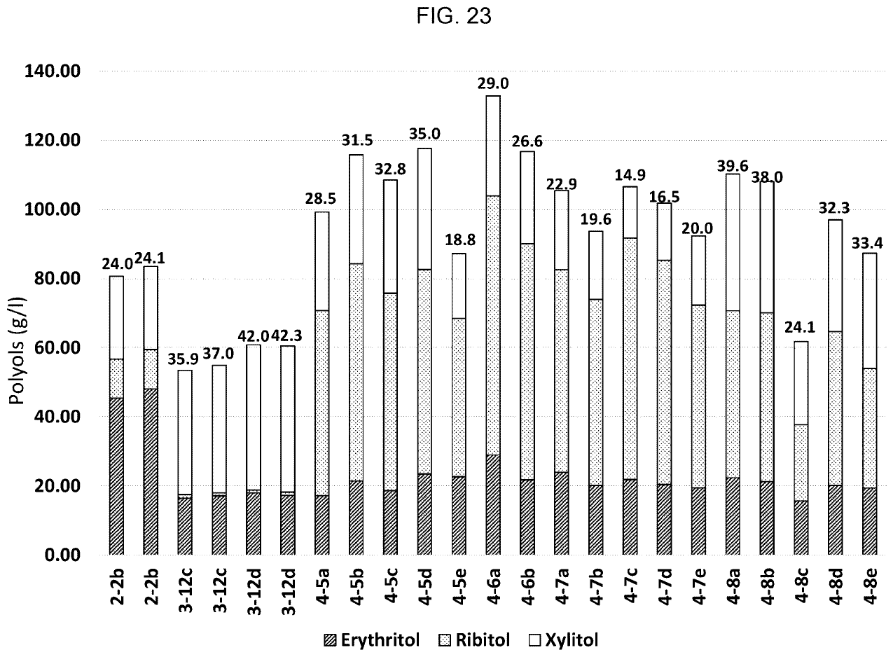

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 48, 72, and 96 hours of incubation. Samples were analyzed for glucose, ribitol, xylitol, erythritol, glycerol, and ethanol by high performance liquid chromatography with refractive index detector. Fermentation results are reported in Table 21 and FIGS. 22 and 23. Results from the 96-hour time point are not shown as some of the reactions consumed the entire glycerol feedstock leading to inaccurate estimates of reaction rate. Results demonstrate that overexpression of the X5PP enzymes of SEQ ID NOs: 198, 199, and 200 increases xylitol titers relative to the parent strain 2-2b and to the control strain expressing RPE and XPDH but lacking an X5PP (strains 3-12c and 3-12d). Results also show that expression of the exogenous X5PP enzyme of SEQ ID NO:201 also increased xylitol titer relative to strains 2-2b, 3-12c, and 3-12d.

TABLE 21

| | Time | | | | | | | Time | | | | | | |
|---|---|---|---|---|---|---|---|---|---|---|---|---|---|---|
| | Point | Fermentation Broth Analyte (g/L) | | | | | | Point | Fermentation Broth Analyte (g/L) | | | | | |
| Strain | (h) | Glucose | Ribitol | Xylitol | Erythritol | Glycerol | Ethanol | (h) | Glucose | Ribitol | Xylitol | Erythritol | Glycerol | Ethanol |
| 2-2b | 48 | 172.91 | 4.03 | 9.78 | 18.73 | 17.54 | 17.58 | 72 | 61.63 | 11.28 | 23.99 | 45.41 | 19.79 | 21.12 |
| 2-2b | 48 | 169.94 | 4.05 | 9.83 | 20.02 | 18.70 | 16.98 | 72 | 59.90 | 11.40 | 24.06 | 48.05 | 20.85 | 20.82 |
| 1-14d | 48 | 158.16 | 21.05 | 6.54 | 10.91 | 14.94 | 22.00 | 72 | 36.09 | 53.72 | 18.09 | 20.62 | 15.53 | 29.51 |
| 1-14d | 48 | 157.33 | 21.43 | 6.63 | 11.17 | 14.76 | 22.80 | 72 | 34.62 | 54.35 | 17.88 | 20.10 | 14.89 | 31.33 |
| 3-12c | 48 | 196.47 | 0.45 | 13.58 | 7.62 | 18.98 | 12.50 | 72 | 109.37 | 0.99 | 35.93 | 16.53 | 21.95 | 17.37 |
| 3-12c | 48 | 199.46 | 0.36 | 13.36 | 7.87 | 20.72 | 11.16 | 72 | 107.68 | 0.83 | 36.96 | 17.17 | 24.55 | 17.18 |
| 3-12d | 48 | 189.61 | 0.23 | 16.14 | 7.48 | 22.14 | 13.65 | 72 | 95.37 | 0.81 | 41.99 | 18.00 | 25.46 | 17.61 |
| 3-12d | 48 | 189.25 | 0.37 | 16.35 | 7.45 | 21.00 | 14.01 | 72 | 93.22 | 0.92 | 42.26 | 17.31 | 23.55 | 19.12 |
| 4-1a | 48 | 156.66 | 0.19 | 37.22 | 12.73 | 14.11 | 19.66 | 72 | 28.19 | 0.42 | 91.71 | 27.02 | 14.09 | 24.11 |
| 4-1b | 48 | 222.95 | 0.69 | 19.92 | 6.73 | 11.40 | 17.92 | 72 | 82.98 | 0.87 | 68.76 | 20.90 | 15.30 | 20.97 |
| 4-1c | 48 | 169.10 | 0.77 | 30.25 | 13.83 | 15.54 | 17.26 | 72 | 36.42 | 2.76 | 77.78 | 32.29 | 17.29 | 23.26 |
| 4-1d | 48 | 176.83 | 0.74 | 29.06 | 13.71 | 15.42 | 14.97 | 72 | 43.70 | 2.54 | 75.34 | 32.81 | 16.51 | 22.73 |
| 4-1e | 48 | 184.82 | 2.72 | 8.44 | 15.98 | 18.27 | 15.44 | 72 | 77.52 | 8.79 | 22.40 | 39.15 | 22.05 | 22.10 |
| 4-2a | 48 | 194.36 | 1.22 | 17.12 | 12.50 | 15.04 | 13.58 | 72 | 63.02 | 5.24 | 48.80 | 33.34 | 16.76 | 25.39 |
| 4-2b | 48 | 194.60 | 0.20 | 25.22 | 9.82 | 13.48 | 13.34 | 72 | 52.65 | 0.69 | 74.57 | 25.24 | 14.22 | 25.86 |
| 4-2c | 48 | 196.79 | 1.33 | 18.60 | 12.59 | 14.65 | 14.32 | 72 | 61.64 | 5.11 | 53.50 | 32.23 | 16.48 | 25.39 |
| 4-2d | 48 | 183.07 | 0.58 | 23.72 | 11.89 | 17.47 | 14.66 | 72 | 42.42 | 1.94 | 69.63 | 30.93 | 18.60 | 24.63 |
| 4-2e | 48 | 185.13 | 0.50 | 17.10 | 7.69 | 20.20 | 15.99 | 72 | 67.79 | 1.21 | 51.10 | 18.41 | 23.84 | 25.17 |
| 4-3a | 48 | 185.83 | 0.45 | 18.85 | 9.09 | 18.50 | 14.93 | 72 | 81.75 | 1.10 | 49.37 | 21.05 | 20.01 | 21.15 |
| 4-3b | 48 | 191.41 | 0.25 | 13.32 | 8.10 | 22.74 | 13.58 | 72 | 87.58 | 0.54 | 41.43 | 19.79 | 24.93 | 20.89 |
| 4-3c | 48 | 192.36 | 0.27 | 13.42 | 8.10 | 23.85 | 12.80 | 72 | 81.47 | 0.64 | 42.57 | 20.53 | 26.27 | 19.40 |
| 4-3d | 48 | 174.50 | 0.98 | 25.52 | 11.42 | 15.82 | 16.39 | 72 | 43.71 | 2.95 | 71.39 | 29.22 | 17.30 | 22.70 |
| 4-3e | 48 | 179.45 | 3.28 | 9.12 | 16.40 | 18.08 | 16.52 | 72 | 70.81 | 10.29 | 23.09 | 40.70 | 20.51 | 23.13 |
| 4-4a | 48 | 168.06 | 0.18 | 33.49 | 11.16 | 11.84 | 18.13 | 72 | 28.55 | 0.42 | 88.19 | 24.52 | 11.46 | 29.19 |
| 4-4b | 48 | 154.62 | 0.13 | 36.82 | 12.45 | 12.77 | 19.98 | 72 | 26.79 | 0.32 | 88.92 | 23.90 | 12.53 | 28.39 |
| 4-4c | 48 | 186.55 | 0.14 | 32.53 | 11.94 | 8.16 | 13.81 | 72 | 27.27 | 0.16 | 91.59 | 31.41 | 9.54 | 25.15 |
| 4-4d | 48 | 158.92 | 0.10 | 31.80 | 12.95 | 14.54 | 20.07 | 72 | 22.74 | 0.11 | 84.67 | 26.35 | 14.28 | 29.46 |
| 4-4e | 48 | 168.10 | 0.41 | 31.21 | 11.08 | 12.35 | 20.29 | 72 | 8.44 | 1.57 | 86.79 | 20.86 | 12.31 | 38.20 |
| 4-5a | 48 | 144.96 | 23.18 | 11.64 | 10.10 | 17.73 | 25.77 | 72 | 24.91 | 53.56 | 28.50 | 17.19 | 17.44 | 34.42 |
| 4-5b | 48 | 136.94 | 26.08 | 11.86 | 12.52 | 16.25 | 25.41 | 72 | 4.16 | 62.84 | 31.55 | 21.42 | 14.75 | 33.56 |
| 4-5c | 48 | 150.01 | 22.92 | 12.02 | 10.17 | 17.74 | 23.83 | 72 | 13.82 | 57.12 | 32.76 | 18.61 | 17.62 | 33.86 |
| 4-5d | 48 | 149.74 | 23.40 | 12.87 | 11.35 | 16.39 | 22.88 | 72 | 9.00 | 59.07 | 35.05 | 23.50 | 16.21 | 30.55 |
| 4-5e | 48 | 171.77 | 15.43 | 5.06 | 9.99 | 21.78 | 17.77 | 72 | 44.04 | 45.76 | 18.77 | 22.71 | 22.29 | 26.80 |
| 4-6a | 48 | 144.33 | 32.18 | 9.84 | 15.51 | 11.73 | 22.41 | 72 | 2.02 | 74.89 | 28.97 | 28.99 | 8.60 | 32.00 |
| 4-6b | 48 | 145.16 | 27.83 | 8.90 | 12.12 | 15.44 | 24.71 | 72 | 3.82 | 68.38 | 26.60 | 21.74 | 13.39 | 35.22 |
| 4-7a | 48 | 166.29 | 21.78 | 6.75 | 10.49 | 17.23 | 20.35 | 72 | 27.16 | 58.56 | 22.86 | 23.98 | 17.28 | 28.03 |
| 4-7b | 48 | 170.31 | 19.76 | 5.83 | 9.45 | 18.58 | 19.45 | 72 | 37.02 | 53.82 | 19.64 | 20.21 | 19.00 | 28.94 |
| 4-7c | 48 | 146.46 | 28.81 | 5.59 | 11.26 | 13.14 | 24.94 | 72 | 14.61 | 69.87 | 14.87 | 21.83 | 12.61 | 33.05 |
| 4-7d | 48 | 149.14 | 26.62 | 6.01 | 10.55 | 16.49 | 24.00 | 72 | 25.21 | 64.86 | 16.50 | 20.42 | 14.67 | 31.92 |
| 4-7e | 48 | 173.91 | 19.37 | 5.74 | 9.15 | 17.99 | 19.08 | 72 | 39.66 | 52.83 | 19.98 | 19.48 | 17.93 | 31.26 |
| 4-8a | 48 | 160.58 | 16.58 | 14.34 | 10.32 | 15.06 | 22.07 | 72 | 5.89 | 48.26 | 39.55 | 22.40 | 14.32 | 33.82 |
| 4-8b | 48 | 158.16 | 17.37 | 14.48 | 10.75 | 14.24 | 22.96 | 72 | 10.29 | 48.78 | 38.04 | 21.24 | 13.57 | 34.39 |
| 4-8c | 48 | 260.46 | 3.85 | 4.24 | 3.64 | 5.83 | 5.10 | 72 | 124.44 | 22.05 | 24.09 | 15.65 | 6.89 | 24.71 |
| 4-8d | 48 | 160.04 | 17.63 | 12.92 | 10.51 | 14.10 | 22.48 | 72 | 33.21 | 44.47 | 32.30 | 20.22 | 13.22 | 33.47 |
| 4-8e | 48 | 204.55 | 10.70 | 10.85 | 7.08 | 8.95 | 15.19 | 72 | 55.44 | 34.55 | 33.38 | 19.41 | 9.14 | 34.25 |

The top xylitol producing strain(s) for each phosphatase enzymes were sequenced. All strains contained two copies of the L. rhamnosus XPDH as expected; however, there was variability in the gene copy number for the phosphatase and RPE2, as outlined in Table 22.

TABLE 22
Gene copy number

| | | Phosphatase | RPE2 |
|---|---|---|---|
| | Phosphatase | gene copy | gene copy |
| Strain | SEQ ID NO: | number | number |
| 4-4c | 200 | 5 | about 7 |
| 4-4b | 200 | 6 | about 6 |
| 4-3d | 199 | about 20 | about 20 |
| 4-2b | 198 | about 14 | about 18 |
| 4-1c | 201 | about 12 | about 14 |
| 4-1a | 201 | about 20 | about 5 |

### Example 19: Shake Flask Fermentation Assay

Strains 1-1, 5-1a-e, 5-2a-e, 5-3a-e, and 5-4a-e were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Fermentation results for the final 96-hour time point are reported in Tables 23 and 24. Results from the 24-, 48-, and 72-hour time points are not shown. Results demonstrate that the phosphatases of SEQ ID NOs: 198-201 do not have the same effect on erythritol production that they do on xylitol production.

TABLE 23
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-1 | 28.76 | 0.97 | 0.16 | 86.04 | 25.18 | 20.71 |
| 1-1 | 29.46 | 1.02 | 0.13 | 87.98 | 25.22 | 16.54 |
| 5-1a | 37.64 | 1.39 | 0.16 | 81.16 | 22.10 | 24.33 |
| 5-1b | 22.23 | 1.01 | 0.12 | 86.80 | 22.83 | 26.94 |
| 5-1c | 10.68 | 4.67 | 0.26 | 91.86 | 28.99 | 26.39 |
| 5-1d | 25.80 | 2.56 | 0.55 | 72.76 | 26.33 | 23.01 |
| 5-1e | 11.57 | 1.24 | 0.22 | 82.89 | 26.66 | 25.25 |
| 5-2a | 29.38 | 5.61 | 0.92 | 75.95 | 25.96 | 22.98 |
| 5-2b | 31.68 | 1.86 | 0.36 | 72.00 | 25.55 | 25.93 |
| 5-2c | 1.99 | 1.54 | 0.20 | 100.53 | 16.16 | 26.39 |
| 5-2d | 68.79 | 0.94 | 0.23 | 67.21 | 21.93 | 16.79 |
| 5-2e | 22.78 | 2.19 | 0.41 | 82.04 | 22.00 | 23.10 |
| 5-3a | 35.81 | 0.85 | 0.14 | 72.21 | 20.19 | 25.78 |
| 5-3b | 62.78 | 0.76 | 0.12 | 61.60 | 24.85 | 20.90 |
| 5-3c | 48.62 | 0.50 | 0.11 | 58.58 | 33.33 | 25.88 |
| 5-3d | 61.74 | 0.55 | 0.12 | 63.04 | 30.94 | 13.31 |
| 5-3e | 67.57 | 0.72 | 0.19 | 58.78 | 27.94 | 18.74 |
| 5-4a | 12.03 | 1.11 | 0.58 | 86.03 | 25.76 | 26.29 |
| 5-4b | 57.19 | 0.72 | 0.11 | 69.49 | 22.89 | 19.95 |
| 5-4c | 28.79 | 1.24 | 0.53 | 82.50 | 23.80 | 23.31 |
| 5-4d | 83.15 | 0.60 | 0.11 | 64.39 | 23.06 | 14.27 |
| 5-4e | 29.45 | 1.36 | 0.57 | 83.47 | 24.09 | 20.32 |

TABLE 24
Erythritol Yield (%)

| | 96-hour | Average |
|---|---|---|
| | Erythritol | Erythritol |
| Strain | Yield | Yield |
| 1-1 | 32.9 | 33.4 |
| 1-1 | 33.8 | |
| 5-1a | 32.2 | 31.0 |
| 5-1b | 32.4 | |
| 5-1c | 32.9 | |
| 5-1d | 27.5 | |
| 5-1e | 29.8 | |
| 5-2a | 29.1 | 30.6 |
| 5-2b | 27.9 | |
| 5-2c | 34.9 | |
| 5-2d | 30.4 | |
| 5-2e | 30.7 | |
| 5-3a | 28.4 | 26.8 |
| 5-3b | 27.1 | |
| 5-3c | 24.3 | |
| 5-3d | 27.6 | |
| 5-3e | 26.4 | |
| 5-4a | 31.0 | 31.1 |
| 5-4b | 29.8 | |
| 5-4c | 31.6 | |
| 5-4d | 31.1 | |
| 5-4e | 32.0 | |

### Example 20: Shake Flask Fermentation Assay

Strains 2-2b, 4-1a, 4-1c, 4-4b, 4-4c, 5-1a-e, 5-2a-e, 5-3a-e, and 5-4a-e were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Fermentation results for the final 96-hour time point are reported in Table 25. Results from the 24-, 48-, and 72-hour time points are not shown.

TABLE 25
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 2-2b | 61.88 | 11.83 | 26.52 | 45.52 | 21.83 | 14.10 |
| 2-2b | 59.17 | 12.13 | 27.31 | 45.75 | 20.98 | 15.58 |
| 4-1a | 11.61 | 0.71 | 105.43 | 30.19 | 11.90 | 17.64 |
| 4-1a | 12.05 | 0.68 | 105.61 | 30.14 | 12.03 | 18.36 |
| 4-1c | 7.29 | 6.27 | 83.29 | 46.44 | 14.11 | 11.48 |
| 4-1c | 9.81 | 6.23 | 82.77 | 44.16 | 14.89 | 15.31 |
| 4-4b | 11.75 | 0.49 | 101.15 | 26.63 | 14.16 | 20.40 |
| 4-4b | 11.32 | 0.47 | 100.91 | 26.02 | 13.80 | 21.13 |
| 4-4c | 0.70 | 0.32 | 112.27 | 34.34 | 7.39 | 19.38 |
| 4-4c | 0.72 | 0.29 | 114.23 | 34.89 | 7.93 | 14.18 |
| 5-5a | 45.61 | 6.13 | 41.81 | 43.00 | 22.58 | 13.60 |
| 5-5b | 51.88 | 12.32 | 27.52 | 48.78 | 20.13 | 17.34 |
| 5-5c | 40.65 | 10.02 | 35.41 | 41.18 | 19.03 | 22.27 |
| 5-5d | 41.92 | 13.06 | 38.53 | 44.82 | 15.98 | 21.55 |
| 5-5e | 27.69 | 14.15 | 30.20 | 61.00 | 22.16 | 10.29 |
| 5-6a | 27.29 | 13.77 | 39.17 | 49.45 | 20.15 | 19.13 |
| 5-6b | 69.40 | 9.85 | 32.12 | 42.35 | 16.92 | 14.69 |
| 5-6c | 48.40 | 5.07 | 59.43 | 37.82 | 9.86 | 20.81 |
| 5-6d | 17.73 | 11.27 | 51.64 | 54.78 | 14.80 | 19.77 |

TABLE 26
96-hour Shake Flask Yield

| | | | Average |
|---|---|---|---|
| | Xylitol | | Xylitol |
| | yield | | Yield |
| Strain | (%) | Description | (%) |
| 2-2b | 11.6 | 2 copies of the sequence encoding the | 11.7 |
| 2-2b | 11.8 | XPDH of SEQ ID NO: 14 | |
| 4-1a | 37.9 | 2 copies of the sequence encoding the | 33.7 |
| 4-1a | 38.0 | XPDH of SEQ ID NO: 14 | |
| 4-1c | 29.5 | 1 copy of the sequence encoding the | |
| 4-1c | 29.5 | RPE of SEQ ID NO: 180 | |
| | | Expression of SEQ ID NO: 201 | |
| 4-4b | 36.4 | 2 copies of the sequence encoding the | 37.7 |
| 4-4b | 36.2 | XPDH of SEQ ID NO: 14 | |
| 4-4c | 38.8 | 1 copy of the sequence encoding the | |
| 4-4c | 39.5 | RPE of SEQ ID NO: 180 | |
| | | Expression of SEQ ID NO: 200 | |
| 5-5a | 17.1 | 2 copies of the sequence encoding the | 14.0 |
| 5-5b | 11.6 | XPDH of SEQ ID NO: 14 | |
| 5-5c | 14.2 | Expression of SEQ ID NO: 201 | |
| 5-5d | 15.5 | | |
| 5-5e | 11.5 | | |
| 5-6a | 14.9 | 2 copies of the sequence encoding the | 18.3 |
| 5-6b | 14.6 | XPDH of SEQ ID NO: 14 | |
| 5-6c | 24.6 | Expression of SEQ ID NO: 200 | |
| 5-6d | 19.0 | | |

This example demonstrates shake flask results comparing two X5PP enzymes (SEQ ID NOs: 200 & 201) with and without expression of the RPE of SEQ ID NO:180. The average xylitol yields in strains containing an overexpressed X5PP but no overexpressed RPE had yields of 14% and 18.3% with phosphatases of SEQ ID Nos: 200 and 201, respectively, in comparison to 11.7% yield in the control strain (see Table 26). These yields are significantly lower than the yields of 33.7% and 37.7% seen in strains containing both the X5PP and RPE expression.

### Example 21: Shake Flask Fermentation Assay

Strains 1-13c, 1-15a, 1-29c, 1-32d, 1-16a, 3-7a, 3-8b, 3-9c, 3-15d, 3-17d, 5-7a-e, 5-8a-e, 5-9a-e, 5-10a-e, and 5-11a-e were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Fermentation results for the final 96-hour time point are reported in Tables 27 and 28. Results from the 24-, 48-, and 72-hour time points are not shown.

TABLE 27
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-13c | 51.94 | 3.64 | 29.42 | 43.98 | 20.46 | 17.61 |
| 1-13c | 50.31 | 3.59 | 30.55 | 42.15 | 19.83 | 18.57 |
| 1-15a | 49.24 | 8.59 | 31.84 | 37.92 | 19.69 | 18.45 |
| 1-15a | 50.44 | 8.52 | 31.38 | 38.82 | 19.35 | 17.81 |
| 1-29c | 19.77 | 4.83 | 29.57 | 53.70 | 21.47 | 20.18 |
| 1-29c | 23.32 | 4.74 | 29.18 | 53.71 | 22.74 | 18.04 |
| 1-32d | 67.72 | 2.53 | 29.81 | 36.53 | 21.10 | 16.27 |
| 1-32d | 68.42 | 2.51 | 29.26 | 36.25 | 20.95 | 16.63 |
| 1-16a | 28.15 | 4.67 | 32.17 | 52.52 | 19.33 | 21.87 |
| 1-16a | 28.50 | 4.67 | 31.40 | 52.40 | 19.02 | 22.04 |
| 3-7a | 58.21 | 1.95 | 35.42 | 32.76 | 22.34 | 16.36 |
| 3-8b | 52.88 | 0.99 | 55.88 | 18.58 | 22.88 | 18.02 |
| 3-9c | 47.89 | 0.92 | 45.08 | 26.03 | 23.12 | 18.84 |
| 3-15d | 68.08 | 0.20 | 47.60 | 18.94 | 22.82 | 17.21 |
| 3-17d | 65.83 | 0.51 | 45.04 | 27.45 | 20.27 | 16.75 |
| 5-7a | 40.99 | 0.19 | 65.76 | 19.53 | 11.59 | 21.96 |
| 5-7b | 23.60 | 0.37 | 69.53 | 21.97 | 14.93 | 25.09 |
| 5-7c | 4.49 | 2.63 | 62.01 | 39.31 | 21.94 | 22.25 |
| 5-7d | 68.06 | 0.26 | 49.74 | 19.58 | 18.96 | 19.87 |
| 5-7e | 58.48 | 0.36 | 1.41 | 45.77 | 27.49 | 25.08 |
| 5-8a | 30.58 | 0.30 | 81.20 | 21.35 | 12.49 | 20.23 |
| 5-8b | 53.75 | 0.19 | 66.34 | 15.91 | 15.11 | 21.20 |
| 5-8c | 39.06 | 2.94 | 58.52 | 28.79 | 15.24 | 20.74 |
| 5-8d | 296.50 | n.a. | 1.59 | 0.80 | 1.32 | 2.96 |
| 5-8e | 53.11 | 0.50 | 60.31 | 16.40 | 15.53 | 21.83 |
| 5-9a | 2.69 | 0.08 | 76.13 | 24.76 | 19.74 | 22.50 |
| 5-9b | 6.92 | 0.11 | 80.34 | 27.56 | 17.95 | 16.22 |
| 5-9c | 70.18 | 0.11 | 48.56 | 18.87 | 21.43 | 17.76 |
| 5-9d | 46.23 | 0.59 | 62.88 | 28.03 | 17.35 | 19.18 |
| 5-9e | 29.45 | 0.15 | 64.75 | 25.19 | 18.21 | 21.49 |
| 5-10a | 46.81 | 0.33 | 1.21 | 49.35 | 36.46 | 20.89 |
| 5-10b | 60.37 | 0.08 | 58.22 | 17.66 | 15.54 | 18.25 |
| 5-10c | 65.25 | 0.32 | 46.92 | 18.44 | 23.33 | 22.04 |
| 5-10d | 49.28 | 0.06 | 66.33 | 18.53 | 11.47 | 25.54 |
| 5-10e | 40.11 | 2.85 | 37.80 | 41.67 | 18.48 | 22.14 |
| 5-11a | 0.62 | 0.29 | 92.69 | 26.42 | 11.08 | 25.28 |
| 5-11b | 0.11 | 0.50 | 91.14 | 28.66 | 11.44 | 24.02 |
| 5-11c | 10.50 | 1.09 | 70.52 | 33.33 | 16.52 | 22.68 |
| 5-11d | 0.99 | 0.46 | 87.05 | 27.17 | 14.00 | 23.53 |
| 5-11e | 0.13 | 1.21 | 83.35 | 27.06 | 10.80 | 27.48 |

TABLE 28
Xylitol Yield (%)

| | 96-hour | XPDH | RPE | | Average |
|---|---|---|---|---|---|
| | Xylitol | SEQ ID | SEQ ID | Phosphatase | Xylitol |
| Strain | Yield* | NO: | NO: | SEQ ID NO: | Yield |
| 1-13c | 12.4 | 12 | none | none | 12.6 |
| 1-13c | 12.7 | | | | |
| 1-15a | 13.2 | 14 | none | none | 13.2 |
| 1-15a | 13.1 | | | | |
| 1-29c | 10.9 | 28 | none | none | 10.9 |
| 1-29c | 10.9 | | | | |
| 1-32d | 13.4 | 31 | none | none | 13.3 |
| 1-32d | 13.2 | | | | |
| 1-16a | 12.3 | 15 | none | none | 12.1 |
| 1-16a | 12.0 | | | | |
| 3-7a | 15.3 | 12 | 180 | none | 15.3 |
| 3-8b | 23.6 | 14 | 180 | none | 23.6 |
| 3-9c | 18.6 | 28 | 180 | none | 18.6 |
| 3-15d | 21.5 | 31 | 180 | none | 21.5 |
| 3-17d | 20.1 | 15 | 180 | none | 20.1 |
| 5-7a | 26.4 | 12 | 180 | 200 | 24.2 |
| 5-7b | 26.1 | | | | |
| 5-7c | 21.7 | | | | |
| 5-7d | 22.4 | | | | |
| 5-7e | 0.6* | | | | |
| 5-8a | 31.3 | 14 | 180 | 200 | 27.0 |
| 5-8b | 28.1 | | | | |
| 5-8c | 23.3 | | | | |
| 5-8d | −24.5* | | | | |
| 5-8e | 25.5 | | | | |
| 5-9a | 26.5 | 28 | 180 | 200 | 25.5 |
| 5-9b | 28.4 | | | | |
| 5-9c | 22.1 | | | | |
| 5-9d | 25.8 | | | | |
| 5-9e | 24.9 | | | | |
| 5-10a | 0.5* | 31 | 180 | 200 | 24.6 |
| 5-10b | 25.4 | | | | |
| 5-10c | 20.9 | | | | |
| 5-10d | 27.6 | | | | |
| 5-10e | 15.1* | | | | |
| 5-11a | 32.0 | 15 | 180 | 200 | 29.5 |
| 5-11b | 31.4 | | | | |
| 5-11c | 25.2 | | | | |
| 5-11d | 30.1 | | | | |
| 5-11e | 28.8 | | | | |

*values in italics are outliers and not used in calculating averages

This shake flask fermentation assay looks at combinations of the phosphatase of SEQ ID NO: 200 with overexpression of RPE2 (SEQ ID NO: 180) and 5 different XPDH enzymes. Table 28 shows the xylitol yields for the strains tested. Significant yield increases were evident for all of the XPDH candidates tested in conjunction with overexpression of RPE2 and the X5PP enzyme of SEQ ID NO:200. Strains with the XPDHs of SEQ ID NOs: 14 and 15 had the highest 96-hour xylitol yields with 27% and 29.5% respectively. Strains tested in this Example all have 1 copy of the indicated XPDH, while strain 2-2b and its progeny have 2 copies of the sequence encoding the XPDH of SEQ ID NO:14, potentially explaining why strains 4-4b and 4-4c have an average yield of 37.7% (see Table 26).

### Example 22: Shake Flask Fermentation Assay

Strains 2-2b, 3-12d, 4-4c, 5-12a-e, 5-13a-d, 5-14a-e, 5-15a-e, 5-16a-e, 5-17a-e, 5-18a-e, 5-19a-d, and 5-20a-d were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

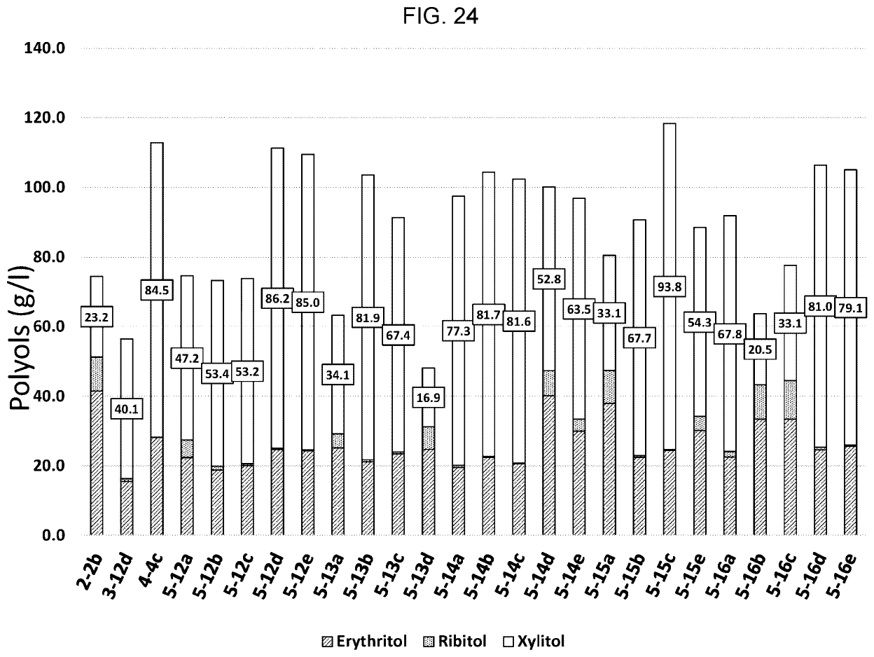

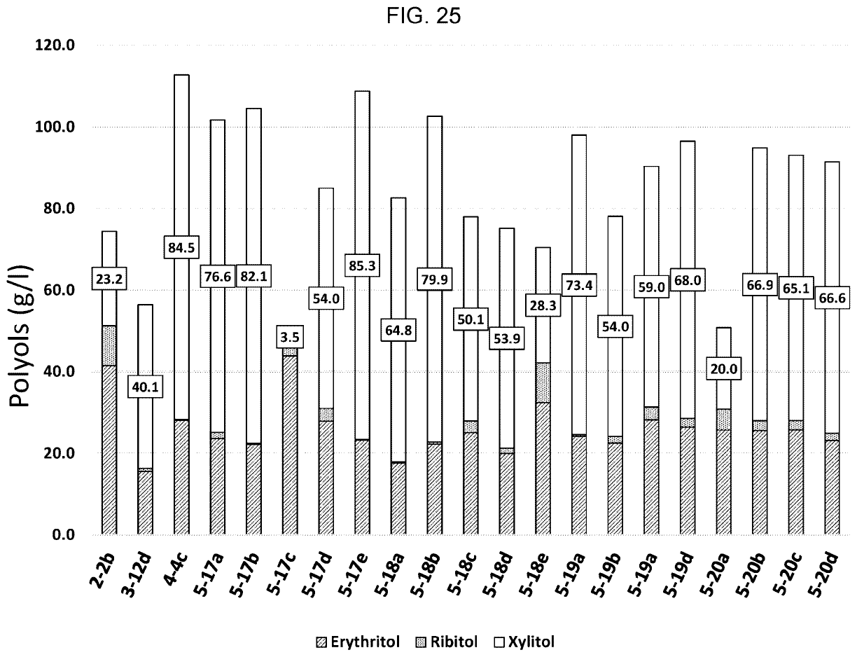

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Fermentation results for the 48- and 72-hour time point are reported in Table 29. Results from the 24- and 96-hour time points are not shown. Titers at the 24-hour time point are low, and after 96 hours, many of the cultures included low or no concentrations of glucose. There was an error in the running of the shake flask with strain 5-15d so no results are reported for this strain. Results for the 72-hour time point are also shown in FIGS. 24 and 25.

TABLE 29
Shake Flask Results

| | Time | | | | | | | Time | | | | | | |
|---|---|---|---|---|---|---|---|---|---|---|---|---|---|---|
| | Point | Fermentation Broth Analyte (g/L) | | | | | | Point | Fermentation Broth Analyte (g/L) | | | | | |
| Strain | (h) | Glucose | Ribitol | Xylitol | Erythritol | Glycerol | Ethanol | (h) | Glucose | Ribitol | Xylitol | Erythritol | Glycerol | Ethanol |
| 2-2b | 48 | 181.56 | 2.83 | 8.92 | 16.59 | 20.47 | 15.46 | 72 | 65.03 | 9.78 | 23.16 | 41.47 | 22.08 | 20.09 |
| 3-12d | 48 | 197.89 | 0.35 | 14.34 | 7.14 | 23.09 | 12.47 | 72 | 95.15 | 0.76 | 40.12 | 15.54 | 25.38 | 19.15 |
| 4-4c | 48 | 206.23 | 0.09 | 26.81 | 10.37 | 8.46 | 11.05 | 72 | 42.03 | 0.15 | 84.54 | 28.09 | 9.85 | 22.75 |
| 5-12a | 48 | 192.18 | 1.38 | 16.09 | 8.84 | 18.54 | 14.82 | 72 | 67.41 | 4.98 | 47.20 | 22.39 | 20.86 | 23.70 |
| 5-12b | 48 | 196.91 | 0.40 | 16.69 | 7.91 | 15.71 | 15.34 | 72 | 69.68 | 1.06 | 53.35 | 18.81 | 16.44 | 25.07 |
| 5-12c | 48 | 203.13 | 0.21 | 14.72 | 8.24 | 20.75 | 12.71 | 72 | 73.16 | 0.51 | 53.25 | 20.03 | 24.22 | 20.55 |
| 5-12d | 48 | 178.40 | 0.19 | 28.56 | 10.73 | 15.56 | 17.20 | 72 | 26.11 | 0.38 | 86.22 | 24.65 | 15.36 | 26.92 |
| 5-12e | 48 | 181.54 | 0.14 | 27.53 | 10.22 | 16.86 | 15.75 | 72 | 30.71 | 0.22 | 84.97 | 24.28 | 16.49 | 25.09 |
| 5-13a | 48 | 203.60 | 0.93 | 10.27 | 9.83 | 20.41 | 12.14 | 72 | 82.12 | 4.01 | 34.07 | 25.18 | 23.02 | 21.67 |
| 5-13b | 48 | 191.83 | 0.20 | 22.57 | 8.10 | 17.48 | 15.71 | 72 | 13.71 | 0.58 | 81.87 | 21.13 | 18.62 | 34.17 |
| 5-13c | 48 | 183.94 | 0.24 | 22.10 | 10.08 | 19.26 | 16.50 | 72 | 48.15 | 0.52 | 67.36 | 23.43 | 19.21 | 23.12 |
| 5-13d | 48 | 230.19 | 1.14 | 4.89 | 9.06 | 14.56 | 9.22 | 72 | 116.43 | 6.51 | 16.88 | 24.70 | 17.41 | 20.57 |
| 5-14a | 48 | 183.71 | 0.24 | 23.28 | 8.15 | 18.92 | 17.14 | 72 | 22.74 | 0.63 | 77.27 | 19.53 | 20.01 | 31.94 |
| 5-14b | 48 | 196.82 | 0.12 | 24.03 | 8.66 | 13.59 | 14.06 | 72 | 39.49 | 0.23 | 81.70 | 22.41 | 12.54 | 27.95 |
| 5-14c | 48 | 198.73 | 0.12 | 23.99 | 7.53 | 15.00 | 13.70 | 72 | 40.90 | 0.22 | 81.59 | 20.56 | 14.76 | 28.94 |
| 5-14d | 48 | 182.03 | 1.74 | 17.62 | 15.73 | 15.73 | 17.64 | 72 | 31.50 | 7.26 | 52.78 | 40.05 | 15.60 | 28.88 |
| 5-14e | 48 | 195.12 | 0.80 | 21.55 | 11.36 | 15.44 | 13.72 | 72 | 48.48 | 3.41 | 63.45 | 29.95 | 16.35 | 23.98 |
| 5-15a | 48 | 190.64 | 2.17 | 11.52 | 13.46 | 16.76 | 15.33 | 72 | 56.55 | 9.41 | 33.11 | 37.95 | 17.94 | 24.45 |
| 5-15b | 48 | 184.00 | 0.20 | 20.81 | 9.20 | 20.30 | 15.50 | 72 | 44.96 | 0.48 | 67.71 | 22.44 | 20.52 | 24.67 |
| 5-15c | 48 | 184.49 | 0.13 | 30.02 | 10.25 | 13.81 | 15.37 | 72 | 18.14 | 0.23 | 93.81 | 24.36 | 13.44 | 29.13 |
| 5-15e | 48 | 187.66 | 1.01 | 18.33 | 11.35 | 18.54 | 15.04 | 72 | 51.84 | 4.05 | 54.28 | 30.10 | 19.22 | 21.55 |
| 5-16a | 48 | 204.67 | 0.38 | 21.25 | 8.07 | 13.71 | 12.75 | 72 | 65.53 | 1.51 | 67.77 | 22.54 | 13.22 | 23.60 |
| 5-16b | 48 | 210.83 | 2.16 | 6.60 | 11.94 | 16.79 | 12.32 | 72 | 84.30 | 9.85 | 20.50 | 33.38 | 21.41 | 23.22 |
| 5-16c | 48 | 185.96 | 2.82 | 11.97 | 12.48 | 19.92 | 15.97 | 72 | 55.75 | 11.07 | 33.14 | 33.40 | 22.65 | 25.64 |
| 5-16d | 48 | 191.55 | 0.25 | 26.45 | 9.43 | 15.51 | 14.36 | 72 | 38.00 | 0.79 | 81.00 | 24.55 | 15.60 | 24.73 |
| 5-16e | 48 | 182.06 | 0.18 | 28.12 | 10.77 | 16.43 | 15.95 | 72 | 45.85 | 0.33 | 79.09 | 25.58 | 16.20 | 21.26 |
| 5-17a | 48 | 188.19 | 0.36 | 25.05 | 9.39 | 16.32 | 15.78 | 72 | 38.82 | 1.47 | 76.60 | 23.64 | 16.21 | 27.36 |
| 5-17b | 48 | 192.07 | 0.17 | 24.17 | 8.38 | 15.36 | 14.80 | 72 | 32.23 | 0.29 | 82.13 | 22.12 | 16.87 | 26.91 |
| 5-17c | 48 | 180.55 | 0.71 | 0.92 | 20.72 | 24.60 | 18.65 | 72 | 61.24 | 2.94 | 3.55 | 43.86 | 30.41 | 30.65 |
| 5-17d | 48 | 182.12 | 0.88 | 20.00 | 11.34 | 17.72 | 15.78 | 72 | 56.15 | 3.18 | 53.97 | 27.85 | 18.25 | 21.94 |
| 5-17e | 48 | 199.84 | 0.11 | 26.91 | 8.42 | 11.45 | 13.14 | 72 | 40.99 | 0.17 | 85.33 | 23.23 | 11.31 | 23.97 |
| 5-18a | 48 | 198.86 | 0.20 | 20.71 | 5.98 | 16.88 | 13.27 | 72 | 66.82 | 0.34 | 64.76 | 17.56 | 18.21 | 21.61 |
| 5-18b | 48 | 196.04 | 0.23 | 26.07 | 8.75 | 12.34 | 13.76 | 72 | 44.77 | 0.55 | 79.88 | 22.21 | 12.97 | 25.07 |
| 5-18c | 48 | 193.04 | 0.71 | 16.65 | 9.58 | 17.19 | 14.45 | 72 | 64.66 | 2.86 | 50.12 | 25.02 | 18.05 | 23.95 |
| 5-18d | 48 | 186.86 | 0.51 | 18.68 | 8.43 | 18.46 | 17.15 | 72 | 67.41 | 1.31 | 53.89 | 19.95 | 17.83 | 23.76 |
| 5-18e | 48 | 193.47 | 2.49 | 9.84 | 12.29 | 18.55 | 14.60 | 72 | 64.37 | 9.76 | 28.27 | 32.42 | 21.37 | 25.55 |
| 5-19a | 48 | 184.98 | 0.21 | 21.74 | 10.10 | 18.13 | 15.77 | 72 | 34.29 | 0.37 | 73.45 | 24.17 | 18.98 | 27.37 |
| 5-19b | 48 | 188.48 | 0.53 | 18.20 | 9.44 | 18.02 | 15.10 | 72 | 62.70 | 1.66 | 53.96 | 22.48 | 18.54 | 23.72 |
| 5-19a | 48 | 200.45 | 0.78 | 17.00 | 9.77 | 15.05 | 12.49 | 72 | 42.77 | 3.17 | 59.00 | 28.18 | 18.40 | 26.03 |
| 5-19d | 48 | 193.11 | 0.51 | 21.60 | 9.98 | 15.35 | 14.58 | 72 | 43.00 | 2.10 | 67.97 | 26.45 | 15.59 | 25.13 |
| 5-20a | 48 | 193.73 | 1.16 | 6.86 | 9.94 | 22.75 | 14.51 | 72 | 90.77 | 5.08 | 20.02 | 25.70 | 25.10 | 21.23 |
| 5-20b | 48 | 196.59 | 0.61 | 21.13 | 9.52 | 14.84 | 13.70 | 72 | 47.01 | 2.42 | 66.88 | 25.53 | 15.11 | 25.33 |
| 5-20c | 48 | 195.96 | 0.56 | 20.41 | 9.66 | 14.58 | 13.94 | 72 | 47.50 | 2.26 | 65.09 | 25.73 | 14.92 | 26.90 |
| 5-20d | 48 | 202.89 | 0.45 | 20.50 | 7.96 | 13.89 | 12.27 | 72 | 55.87 | 1.72 | 66.58 | 23.15 | 14.54 | 24.25 |

As shown in FIGS. 24 and 25, and the results shown in Table 29, strains expressing the X5PP enzymes of SEQ ID NOs: 201-209 demonstrated increased titers of xylitol compared to the control strain 2-2b as well as a strain expressing RPE (3-12d). Strains expressing the X5PP enzymes of SEQ ID NOs: 201-207 also had at least one sister isolate with xylitol titers equivalent to or above the xylitol titer of strain 4-4c, which expresses the X5PP of SEQ ID NO:200. There is also sister to sister variability in several of the strains assayed. As demonstrated in several examples above, while a strain is PCR verified to contain the desired polynucleotide sequence, said sequence may not be integrated at the correct locus, it may have had multiple copies of the sequence integrated into its genome, or a frameshift or other mutation caused an individual sister to vary from the others. The results here suggest that similar transformation occurrences are present in these sisters, but the results do demonstrate the effectiveness of the indicated X5PP enzymes.

For example, while PCR verification indicated that the transformed polynucleotide sequence was present in the indicated strains, further whole genome sequencing analysis indicated that for strains 5-12a-e, 5-15a-c, and 5-15e, the sequence was integrated in more than one copy and/or was not correctly integrated at the ER3 locus. Whole genome sequencing results for these strains are reported below in Table 30. Results show the sister-to-sister variability is likely due to differences in gene copy number (of the XPDH, RPE2, and/or the phosphatase genes) and integration loci, but even with copy number and integration variability the effectiveness of the indicated X5PP enzymes is consistent.

TABLE 30

| | | XPDH gene | RPE2 gene | Phosphatase gene | | | |
|---|---|---|---|---|---|---|---|
| | | (Encoding | (Encoding | (Encoding | (Encoding | (Encoding | |
| | WT | SEQ ID | SEQ ID | SEQ ID | SEQ ID | SEQ ID | |
| Strain | ER3 | NO: 14) | NO: 180) | NO: 202) | NO: 204) | NO: 213) | Notes |
| 1-1 | 2 | 0 | 1 | 0 | | | The wild-type strain natively has 2 copies |
| | | | | | | | of the ER3 gene and 1 copy of the RPE2 gene |
| 5-12a | 2 | 2 | 3 | 2 | | | |
| 5-12b | 2 | 2 | 2 | 2 | | | |
| 5-12c | 2 | 1 | 2 | 1 | | | The construct was designed to insert the |
| | | | | | | | sequence encoding SEQ ID NO: 202 at the ER3 |
| | | | | | | | locus, however the coding sequence replaced 1 |
| | | | | | | | of the two copies of the XPDH coding sequence |
| | | | | | | | at the ER1 locus. |
| 5-12d | 2 | 2 | 4 | 7 | | | An 8^{th} phosphatase gene was |
| | | | | | | | present but was missing its terminator |
| 5-12e | 2 | 2 | 5 | 4 | | | |
| 5-15a | 1 | 2 | 2 | | 1 | | One allele of the ER3 gene was replaced with |
| | | | | | | | the sequence encoding SEQ ID NO: 204 |
| 5-15b | 2 | 2 | 3 | | 1 | | The construct was designed to insert the |
| | | | | | | | sequence encoding SEQ ID NO: 202 at the ER3 |
| | | | | | | | locus, however the coding sequence was |
| | | | | | | | integrated between the first and second copies |
| | | | | | | | of the sequence encoding the XPDH at ER1 |
| 5-15c | 2 | 2 | 7 | | 8 | | 7 full RPE2 genes, 1 RPE2 gene with a gap, |
| | | | | | | | and 1 RPE2 gene missing the promoter |
| 5-15e | 2 | 2 | 7 | 3 | | | 3 active phosphatase genes, 2 additional |
| | | | | | | | phosphatase genes missing half of the gene |
| 5-26b | 2 | 2 | 2 | | | 1 | 1 active phosphatase gene, 1 phosphatase |
| | | | | | | | missing half of promoter |
| | | | | | | | The construct designed to insert at the ER3 locus |
| | | | | | | | replaced the 2^{nd} half of the ER1 locus |

### Example 23: Shake Flask Fermentation Assay

Strains 2-2b, 4-4b, 4-4c, 5-21a-c, 5-22, 5-23a-c, 5-24a-b, 5-25a-e, 5-26a-d, 5-27, 5-28a-d, 5-29, 5-30a-b, 5-31a-b, 5-32a-c, and 5-33 were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

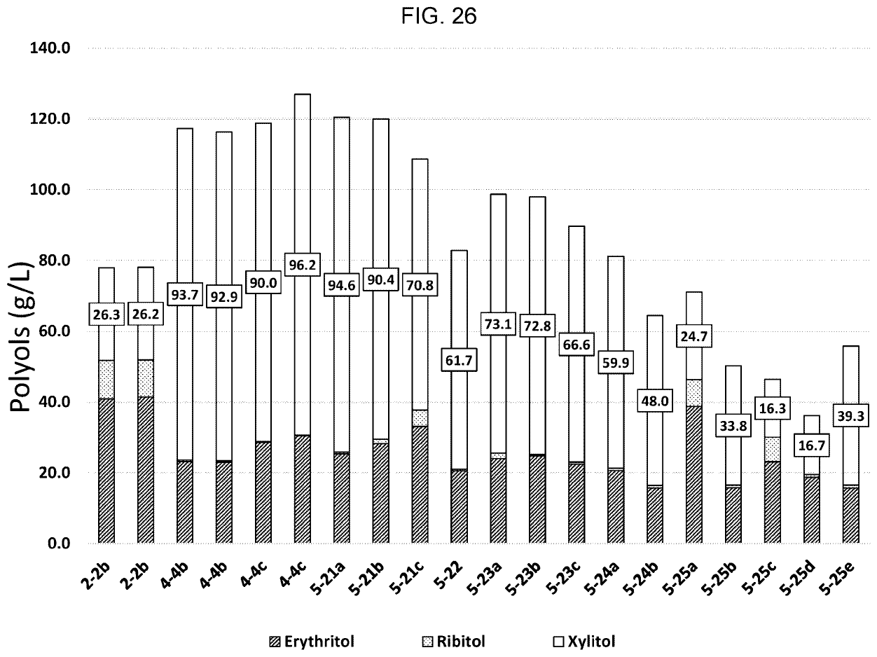

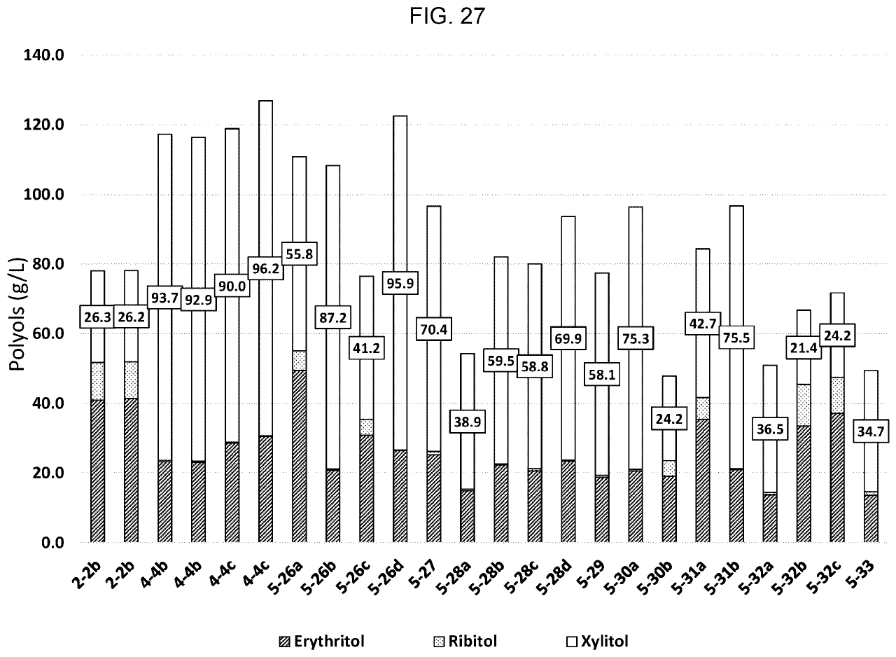

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Fermentation results for the 48- and 72-hour time point are reported in Table 31. Results from the 24- and 96-hour time points are not shown. Titers at the 24-hour time point are low, and after 96 hours, many of the cultures included low or no concentrations of glucose. Results from the 72-hour time point are also shown in FIGS. 26 and 27.

TABLE 31
Shake Flask Results

| | Time | | | | | | | Time | | | | | | |
|---|---|---|---|---|---|---|---|---|---|---|---|---|---|---|
| | Point | Fermentation Broth Analyte (g/L) | | | | | | Point | Fermentation Broth Analyte (g/L) | | | | | |
| Strain | (h) | Glucose | Ribitol | Xylitol | Erythritol | Glycerol | Ethanol | (h) | Glucose | Ribitol | Xylitol | Erythritol | Glycerol | Ethanol |
| 2-2b | 48 | 163.34 | 3.70 | 11.11 | 18.38 | 18.76 | 18.53 | 72 | 64.44 | 10.78 | 26.29 | 40.92 | 21.41 | 23.30 |
| 2-2b | 48 | 160.66 | 3.72 | 11.38 | 18.82 | 18.34 | 19.12 | 72 | 68.06 | 10.46 | 26.22 | 41.40 | 20.76 | 23.92 |
| 4-4b | 48 | 141.57 | 0.17 | 39.90 | 13.61 | 13.57 | 20.73 | 72 | 19.04 | 0.44 | 93.67 | 23.19 | 13.16 | 30.22 |
| 4-4b | 48 | 144.90 | 0.16 | 39.62 | 13.41 | 13.17 | 20.94 | 72 | 19.81 | 0.41 | 92.88 | 23.02 | 12.59 | 31.64 |
| 4-4c | 48 | 170.80 | 0.10 | 34.67 | 12.68 | 8.10 | 15.87 | 72 | 36.57 | 0.18 | 90.03 | 28.64 | 8.73 | 25.91 |
| 4-4c | 48 | 173.75 | 0.10 | 36.09 | 13.23 | 8.93 | 14.57 | 72 | 26.50 | 0.19 | 96.21 | 30.54 | 9.79 | 25.28 |
| 5-21a | 48 | 153.61 | 0.16 | 37.67 | 12.20 | 13.75 | 18.80 | 72 | 24.40 | 0.47 | 94.59 | 25.37 | 14.09 | 27.66 |
| 5-21b | 48 | 149.46 | 0.39 | 36.48 | 13.74 | 14.31 | 19.17 | 72 | 24.73 | 1.25 | 90.43 | 28.26 | 14.54 | 27.42 |
| 5-21c | 48 | 135.38 | 1.66 | 29.40 | 16.42 | 20.35 | 21.62 | 72 | 20.16 | 4.66 | 70.85 | 33.08 | 21.66 | 28.58 |
| 5-22 | 48 | 181.90 | 0.18 | 20.11 | 8.92 | 22.92 | 13.56 | 72 | 61.80 | 0.49 | 61.71 | 20.59 | 26.04 | 25.74 |
| 5-23a | 48 | 138.22 | 0.52 | 29.35 | 12.29 | 25.77 | 20.36 | 72 | 24.02 | 1.56 | 73.09 | 24.04 | 27.78 | 28.16 |
| 5-23b | 48 | 170.61 | 0.18 | 27.04 | 11.67 | 17.54 | 17.32 | 72 | 52.80 | 0.41 | 72.76 | 24.79 | 17.81 | 23.02 |
| 5-23c | 48 | 159.61 | 0.23 | 25.84 | 10.53 | 18.51 | 17.36 | 72 | 57.19 | 0.59 | 66.64 | 22.44 | 19.58 | 22.83 |
| 5-24a | 48 | 159.71 | 0.27 | 24.69 | 10.18 | 18.14 | 18.47 | 72 | 66.35 | 0.64 | 59.87 | 20.68 | 19.52 | 23.63 |
| 5-24b | 48 | 174.78 | 0.29 | 16.91 | 7.72 | 20.40 | 16.37 | 72 | 76.03 | 0.74 | 47.96 | 15.72 | 23.73 | 26.09 |
| 5-25a | 48 | 169.80 | 2.30 | 10.38 | 16.04 | 18.76 | 17.71 | 72 | 74.09 | 7.58 | 24.70 | 38.76 | 21.56 | 23.19 |
| 5-25b | 48 | 182.88 | 0.32 | 13.71 | 8.73 | 23.29 | 13.67 | 72 | 108.49 | 0.71 | 33.76 | 15.81 | 29.36 | 17.42 |
| 5-25c | 48 | 214.31 | 1.32 | 5.57 | 8.91 | 14.63 | 10.67 | 72 | 122.23 | 7.01 | 16.33 | 23.11 | 18.50 | 22.12 |
| 5-25d | 48 | 181.93 | 0.34 | 7.74 | 10.86 | 28.34 | 12.96 | 72 | 120.11 | 0.73 | 16.65 | 18.76 | 42.72 | 14.20 |
| 5-25e | 48 | 177.67 | 0.40 | 16.79 | 8.09 | 18.84 | 15.07 | 72 | 104.41 | 0.86 | 39.29 | 15.69 | 21.25 | 19.92 |
| 5-26a | 48 | 147.80 | 1.64 | 24.61 | 20.41 | 15.80 | 19.33 | 72 | 32.51 | 5.61 | 55.82 | 49.46 | 16.00 | 21.16 |
| 5-26b | 48 | 150.28 | 0.16 | 37.66 | 10.57 | 13.91 | 20.17 | 72 | 44.12 | 0.34 | 87.19 | 20.80 | 14.09 | 25.11 |
| 5-26c | 48 | 152.24 | 1.99 | 20.53 | 16.30 | 18.41 | 20.17 | 72 | 81.78 | 4.48 | 41.17 | 30.88 | 21.08 | 19.23 |
| 5-26d | 48 | 153.64 | 0.07 | 40.85 | 13.46 | 11.53 | 17.69 | 72 | 35.00 | 0.12 | 95.93 | 26.48 | 10.99 | 24.18 |
| 5-27 | 48 | 199.92 | 0.22 | 23.07 | 10.50 | 12.68 | 10.82 | 72 | 68.88 | 0.90 | 70.44 | 25.27 | 13.77 | 24.66 |
| 5-28a | 48 | 202.09 | 0.15 | 11.55 | 5.91 | 18.51 | 10.93 | 72 | 104.68 | 0.44 | 38.94 | 14.90 | 21.23 | 19.69 |
| 5-28b | 48 | 161.10 | 0.11 | 23.75 | 10.85 | 18.03 | 17.59 | 72 | 63.20 | 0.23 | 59.50 | 22.36 | 19.03 | 22.15 |
| 5-28c | 48 | 163.18 | 0.24 | 23.43 | 10.19 | 17.68 | 18.05 | 72 | 65.16 | 0.57 | 58.77 | 20.68 | 18.60 | 24.57 |
| 5-28d | 48 | 156.92 | 0.15 | 25.71 | 11.63 | 18.67 | 18.62 | 72 | 44.06 | 0.30 | 69.94 | 23.41 | 20.65 | 28.44 |
| 5-29 | 48 | 191.41 | 0.20 | 18.55 | 8.17 | 15.15 | 14.02 | 72 | 76.66 | 0.53 | 58.10 | 18.79 | 16.39 | 25.84 |
| 5-30a | 48 | 149.86 | 0.22 | 29.78 | 10.37 | 16.96 | 20.34 | 72 | 35.04 | 0.46 | 75.34 | 20.59 | 17.53 | 28.04 |
| 5-30b | 48 | 194.96 | 1.33 | 9.46 | 8.05 | 13.40 | 16.43 | 72 | 109.57 | 4.49 | 24.24 | 19.09 | 15.64 | 25.32 |
| 5-31a | 48 | 144.16 | 2.29 | 21.02 | 17.52 | 19.55 | 21.23 | 72 | 62.82 | 6.26 | 42.71 | 35.37 | 18.16 | 24.51 |
| 5-31b | 48 | 141.36 | 0.09 | 30.32 | 12.44 | 15.59 | 24.26 | 72 | 26.17 | 0.19 | 75.48 | 21.03 | 15.92 | 34.10 |
| 5-32a | 48 | 182.59 | 0.32 | 15.34 | 7.23 | 18.61 | 14.79 | 72 | 112.84 | 0.74 | 36.47 | 13.73 | 21.17 | 19.23 |
| 5-32b | 48 | 166.23 | 3.47 | 8.64 | 14.86 | 20.88 | 17.87 | 72 | 68.72 | 11.86 | 21.35 | 33.54 | 25.76 | 26.57 |
| 5-32c | 48 | 155.71 | 3.61 | 10.59 | 16.79 | 18.98 | 19.99 | 72 | 65.34 | 10.35 | 24.24 | 37.15 | 21.12 | 25.28 |
| 5-33 | 48 | 181.57 | 0.44 | 14.14 | 7.18 | 20.16 | 16.08 | 72 | 107.11 | 1.04 | 34.72 | 13.62 | 23.17 | 20.95 |

As shown in FIGS. 26 and 27, and the results shown in Table 31, strains expressing the X5PP enzymes of SEQ ID NOs: 210, 211, 213, 214, and 189 demonstrated increased titers of xylitol compared to the control strain 2-2b. Strains expressing the X5PP enzymes of SEQ ID NOs: 210 and 213 also had at least one sister isolate with xylitol titers equivalent to or above the xylitol titer of strains 4-4b and 4-4c, which expresses the X5PP of SEQ ID NO:200. There is also sister to sister variability in several of the strains assayed. As demonstrated in several examples above, while a strain is PCR verified to contain the desired polynucleotide sequence, said sequence may not be integrated at the correct loci, it may have had multiple copies of the sequence integrated into its genome, or a frameshift or other mutation caused an individual sister to vary from the others. The results here suggest that similar transformation occurrences are present in these sisters, but the results do demonstrate the effectiveness of the indicated X5PP enzymes.

### Example 24: Shake Flask Fermentation Assay

Strains 2-2b, 4-4b, 4-4c, 5-34a-c, 5-35a-c, 5-36a-b, 5-37a-c, 5-38, 5-39a-b, 5-40a-c, 5-41a-b, 5-42a-d, 5-43a-f, 5-44a-b, 5-45a-d, 5-46, and 5-47a-e were run in shake flasks to assess glucose consumption as well as ribitol, erythritol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

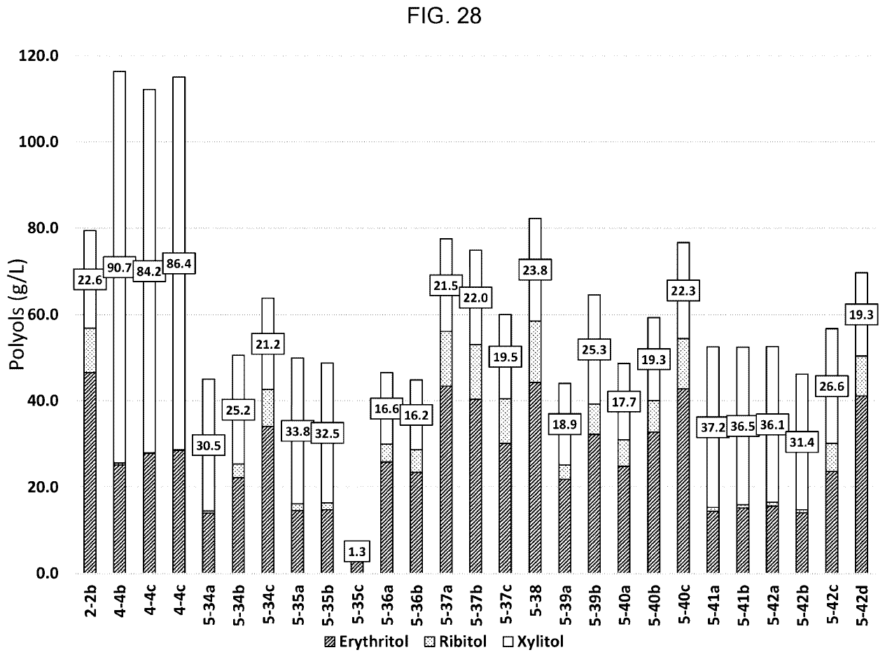

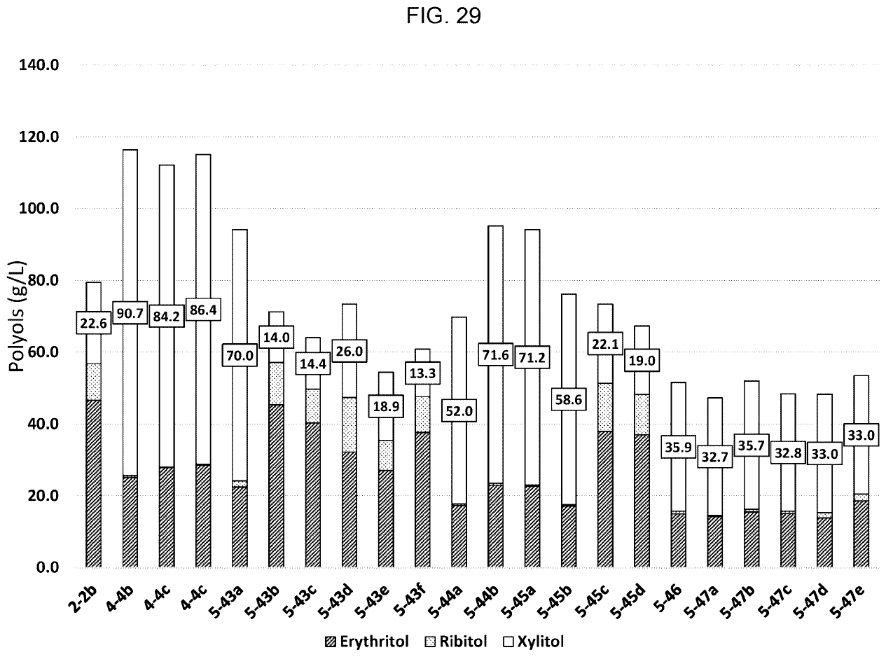

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Fermentation results for the 48- and 72-hour time point are reported in Table 32. Results from the 24- and 96-hour time points are not shown. Titers at the 24-hour time point are low, and after 96 hours, many of the cultures included low or no concentrations of glucose. Results from the 72-hour time point are also shown in FIGS. 28 and 29.

TABLE 32
Shake Flask Results

| | Time | | | | | | | Time | | | | | | |
|---|---|---|---|---|---|---|---|---|---|---|---|---|---|---|
| | Point | Fermentation Broth Analyte (g/L) | | | | | | Point | Fermentation Broth Analyte (g/L) | | | | | |
| Strain | (h) | Glucose | Ribitol | Xylitol | Erythritol | Glycerol | Ethanol | (h) | Glucose | Ribitol | Xylitol | Erythritol | Glycerol | Ethanol |
| 2-2b | 48 | 149.52 | 4.86 | 12.24 | 23.65 | 22.80 | 18.80 | 72 | 68.85 | 10.26 | 22.64 | 46.57 | 24.70 | 18.64 |
| 4-4b | 48 | 133.15 | 0.26 | 47.52 | 16.29 | 13.94 | 23.04 | 72 | 32.97 | 0.53 | 90.70 | 25.10 | 13.24 | 28.13 |
| 4-4c | 48 | 164.19 | 0.13 | 39.96 | 15.00 | 9.65 | 16.53 | 72 | 45.24 | 0.17 | 84.22 | 27.79 | 10.20 | 24.36 |
| 4-4c | 48 | 162.27 | 0.16 | 39.54 | 14.93 | 9.36 | 16.55 | 72 | 45.70 | 0.17 | 86.38 | 28.52 | 10.15 | 25.24 |
| 5-34a | 48 | 192.02 | 0.28 | 12.79 | 7.79 | 20.60 | 15.20 | 72 | 116.21 | 0.49 | 30.55 | 13.97 | 23.08 | 22.58 |
| 5-34b | 48 | 203.18 | 0.93 | 9.62 | 9.97 | 20.58 | 12.32 | 72 | 118.89 | 3.21 | 25.19 | 22.14 | 25.09 | 18.69 |
| 5-34c | 48 | 173.46 | 3.43 | 10.11 | 16.55 | 18.52 | 18.53 | 72 | 93.45 | 8.52 | 21.20 | 34.10 | 19.92 | 23.62 |
| 5-35a | 48 | 185.55 | 0.73 | 14.29 | 8.18 | 22.43 | 14.27 | 72 | 105.28 | 1.63 | 33.79 | 14.52 | 26.54 | 22.31 |
| 5-35b | 48 | 199.38 | 0.69 | 13.08 | 7.96 | 17.74 | 12.10 | 72 | 114.23 | 1.61 | 32.46 | 14.71 | 23.37 | 21.48 |
| 5-35c | 48 | 283.98 | 0.05 | 0.89 | 3.82 | 5.91 | 1.64 | 72 | 281.19 | 0.11 | 1.26 | 4.40 | 6.66 | 1.87 |
| 5-36a | 48 | 193.58 | 1.50 | 7.57 | 12.86 | 22.28 | 13.12 | 72 | 121.31 | 4.10 | 16.61 | 25.84 | 25.33 | 17.70 |
| 5-36b | 48 | 199.48 | 1.92 | 8.34 | 10.55 | 17.52 | 12.95 | 72 | 132.15 | 5.24 | 16.16 | 23.43 | 20.19 | 18.31 |
| 5-37a | 48 | 153.24 | 5.74 | 11.03 | 22.17 | 23.37 | 17.90 | 72 | 69.71 | 12.66 | 21.49 | 43.40 | 26.75 | 22.84 |
| 5-37b | 48 | 161.28 | 5.25 | 11.02 | 18.90 | 21.03 | 18.25 | 72 | 75.14 | 12.63 | 21.97 | 40.34 | 24.50 | 23.38 |
| 5-37c | 48 | 176.92 | 3.35 | 8.77 | 13.98 | 21.42 | 16.74 | 72 | 91.58 | 10.36 | 19.52 | 30.11 | 25.80 | 24.29 |
| 5-38 | 48 | 141.76 | 6.70 | 13.26 | 20.47 | 23.14 | 19.41 | 72 | 56.39 | 14.23 | 23.79 | 44.27 | 26.07 | 20.14 |
| 5-39a | 48 | 202.34 | 1.07 | 7.51 | 10.48 | 21.86 | 13.07 | 72 | 122.05 | 3.30 | 18.94 | 21.82 | 25.53 | 21.30 |
| 5-39b | 48 | 168.10 | 2.94 | 12.61 | 16.22 | 20.10 | 17.41 | 72 | 89.79 | 7.06 | 25.29 | 32.22 | 21.73 | 22.31 |
| 5-40a | 48 | 216.02 | 1.63 | 6.77 | 10.90 | 10.57 | 10.68 | 72 | 133.28 | 6.19 | 17.70 | 24.79 | 13.78 | 19.37 |
| 5-40b | 48 | 178.82 | 2.55 | 8.80 | 15.37 | 19.90 | 15.35 | 72 | 96.32 | 7.29 | 19.25 | 32.74 | 22.60 | 21.87 |
| 5-40c | 48 | 154.60 | 5.18 | 10.79 | 20.95 | 24.23 | 18.31 | 72 | 65.63 | 11.66 | 22.29 | 42.77 | 26.68 | 23.44 |
| 5-41a | 48 | 181.60 | 0.53 | 17.49 | 7.73 | 17.94 | 15.73 | 72 | 109.00 | 0.91 | 37.21 | 14.38 | 20.55 | 22.22 |
| 5-41b | 48 | 178.72 | 0.45 | 17.69 | 8.75 | 20.88 | 14.79 | 72 | 114.27 | 0.80 | 36.52 | 15.13 | 23.18 | 18.34 |
| 5-42a | 48 | 175.08 | 0.48 | 18.12 | 9.15 | 22.90 | 14.42 | 72 | 113.14 | 0.88 | 36.10 | 15.60 | 25.55 | 17.70 |
| 5-42b | 48 | 192.57 | 0.42 | 13.93 | 7.99 | 21.70 | 11.32 | 72 | 127.32 | 0.72 | 31.43 | 14.03 | 25.10 | 16.39 |
| 5-42c | 48 | 181.53 | 2.18 | 10.26 | 12.56 | 26.69 | 14.23 | 72 | 92.15 | 6.50 | 26.62 | 23.62 | 34.87 | 19.90 |
| 5-42d | 48 | 176.30 | 3.54 | 8.46 | 20.07 | 20.04 | 15.10 | 72 | 84.12 | 9.22 | 19.33 | 41.15 | 24.39 | 22.55 |
| 5-43a | 48 | 166.75 | 0.66 | 27.72 | 12.13 | 15.36 | 19.51 | 72 | 43.19 | 1.77 | 70.01 | 22.38 | 16.54 | 34.65 |
| 5-43b | 48 | 180.46 | 5.26 | 5.48 | 23.53 | 14.20 | 14.40 | 72 | 89.20 | 11.84 | 14.03 | 45.34 | 18.51 | 22.33 |
| 5-43c | 48 | 168.11 | 3.52 | 6.46 | 20.23 | 22.82 | 16.34 | 72 | 81.83 | 9.40 | 14.42 | 40.24 | 28.08 | 23.03 |
| 5-43d | 48 | 142.99 | 6.54 | 12.81 | 16.37 | 26.03 | 23.28 | 72 | 54.00 | 15.16 | 26.02 | 32.20 | 31.41 | 28.05 |
| 5-43e | 48 | 184.67 | 3.44 | 9.30 | 12.50 | 12.22 | 18.30 | 72 | 108.41 | 8.41 | 18.95 | 27.06 | 14.70 | 25.02 |
| 5-43f | 48 | 185.52 | 4.14 | 6.15 | 18.04 | 15.35 | 15.82 | 72 | 101.15 | 9.96 | 13.27 | 37.60 | 18.00 | 22.50 |
| 5-44a | 48 | 157.63 | 0.12 | 24.88 | 9.04 | 20.46 | 18.76 | 72 | 83.07 | 0.45 | 52.05 | 17.26 | 21.19 | 24.39 |
| 5-44b | 48 | 154.67 | 0.30 | 33.62 | 12.36 | 14.36 | 19.20 | 72 | 57.60 | 0.62 | 71.62 | 22.90 | 14.24 | 27.62 |
| 5-45a | 48 | 153.87 | 0.22 | 33.15 | 12.28 | 16.19 | 18.34 | 72 | 57.67 | 0.40 | 71.20 | 22.57 | 16.27 | 25.63 |
| 5-45b | 48 | 139.89 | 0.22 | 31.39 | 10.53 | 19.51 | 22.87 | 72 | 68.27 | 0.41 | 58.58 | 17.16 | 19.92 | 24.97 |
| 5-45c | 48 | 164.34 | 5.25 | 11.02 | 17.56 | 23.56 | 17.26 | 72 | 76.51 | 13.46 | 22.06 | 37.89 | 27.78 | 22.96 |
| 5-45d | 48 | 170.10 | 4.15 | 9.11 | 16.99 | 23.60 | 16.70 | 72 | 83.93 | 11.27 | 19.03 | 36.99 | 27.43 | 22.91 |
| 5-46 | 48 | 174.83 | 0.31 | 17.71 | 8.64 | 22.75 | 14.30 | 72 | 114.21 | 0.74 | 35.88 | 14.94 | 25.46 | 17.75 |
| 5-47a | 48 | 177.67 | 0.19 | 15.23 | 8.56 | 25.33 | 14.95 | 72 | 114.35 | 0.32 | 32.72 | 14.21 | 28.60 | 18.05 |
| 5-47b | 48 | 173.49 | 0.45 | 18.52 | 9.08 | 23.06 | 14.26 | 72 | 114.55 | 0.74 | 35.73 | 15.47 | 25.70 | 16.14 |
| 5-47c | 48 | 181.88 | 0.36 | 16.15 | 8.69 | 22.38 | 13.51 | 72 | 121.04 | 0.64 | 32.77 | 15.02 | 25.05 | 17.10 |
| 5-47d | 48 | 181.90 | 0.75 | 15.22 | 7.95 | 20.76 | 14.93 | 72 | 105.22 | 1.47 | 33.02 | 13.80 | 24.71 | 22.77 |
| 5-47e | 48 | 174.77 | 0.86 | 16.07 | 10.15 | 21.67 | 15.29 | 72 | 106.38 | 1.91 | 32.96 | 18.56 | 24.00 | 18.88 |

As shown in FIGS. 28 and 29, and the results shown in Table 32, strains expressing the X5PP enzymes of SEQ ID NOs: 221, 222, and 188 demonstrated increased titers of xylitol compared to the control strain 2-2b. None of the strains tested had xylitol titers equivalent to or above the xylitol titer of strains 4-4b and 4-4c, which expresses the X5PP of SEQ ID NO:200. There is also sister to sister variability in several of the strains assayed. As demonstrated in several examples above, while a strain is PCR verified to contain the desired polynucleotide sequence, said sequence may not be integrated at the correct loci, it may have had multiple copies of the sequence integrated into its genome, or a frameshift or other mutation caused an individual sister to vary from the others. The results here suggest that similar transformation occurrences are present in these sisters, but the results do demonstrate the effectiveness of the indicated X5PP enzymes.

### Example 25: Shake Flask Fermentation Assay

Strains 4-4b, 4-4c, 5-12d, 5-12e, 5-14b, 5-14c, 5-15c, 5-16d, 5-16e, 5-17e, 5-21a, 5-21b, 5-26b, and 5-26d were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

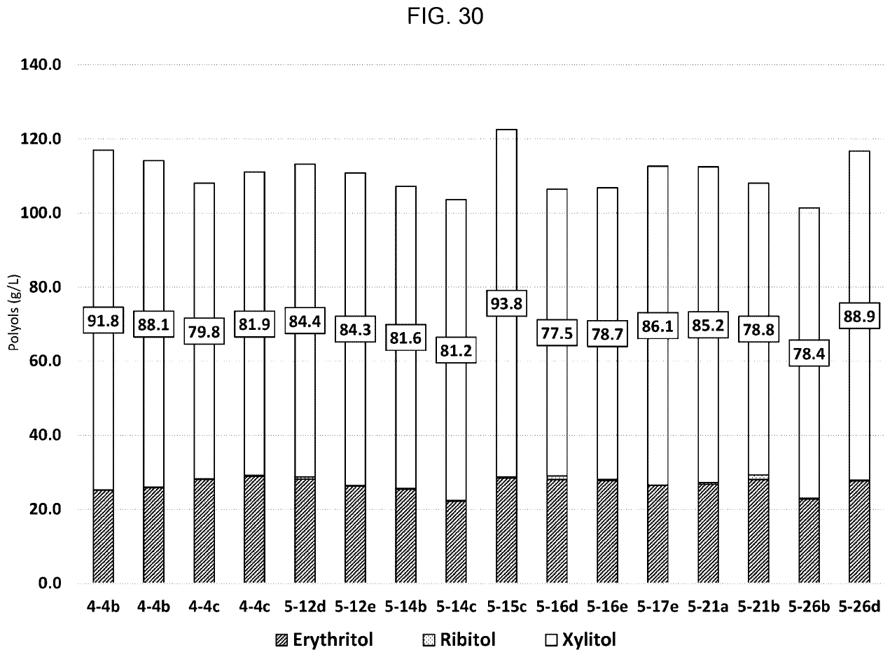

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Fermentation results for the 48- and 72-hour time point are reported in Table 33. Results from the 24- and 96-hour time points are not shown. Titers at the 24-hour time point are low, and after 96 hours, many of the cultures included low or no concentrations of glucose. Results for the 72-hour time point are also shown in FIG. 30.

TABLE 33
Shake Flask Results

| | Time | | | | | | | Time | | | | | | |
|---|---|---|---|---|---|---|---|---|---|---|---|---|---|---|
| | Point | Fermentation Broth Analyte (g/L) | | | | | | Point | Fermentation Broth Analyte (g/L) | | | | | |
| Strain | (h) | Glucose | Ribitol | Xylitol | Erythritol | Glycerol | Ethanol | (h) | Glucose | Ribitol | Xylitol | Erythritol | Glycerol | Ethanol |
| 4-4b | 48 | 168.42 | 0.17 | 35.53 | 12.01 | 13.90 | 18.13 | 72 | 28.34 | n.a. | 91.75 | 25.17 | 15.41 | 31.37 |
| 4-4b | 48 | 166.93 | 0.14 | 35.13 | 11.82 | 13.79 | 18.57 | 72 | 29.52 | 0.15 | 88.12 | 25.81 | 15.20 | 31.45 |
| 4-4c | 48 | 199.83 | 0.11 | 28.28 | 10.23 | 7.89 | 12.18 | 72 | 55.20 | 0.15 | 79.83 | 28.02 | 9.77 | 26.15 |
| 4-4c | 48 | 198.08 | 0.07 | 29.17 | 10.64 | 8.04 | 12.78 | 72 | 54.52 | 0.16 | 81.90 | 28.91 | 10.50 | 26.21 |
| 5-12d | 48 | 165.75 | 0.14 | 31.61 | 11.63 | 15.85 | 18.50 | 72 | 32.63 | 0.56 | 84.44 | 28.16 | 17.05 | 28.45 |
| 5-12e | 48 | 173.48 | 0.14 | 31.45 | 10.86 | 16.83 | 17.61 | 72 | 36.33 | 0.23 | 84.29 | 26.26 | 18.72 | 27.39 |
| 5-14b | 48 | 186.76 | 0.13 | 27.96 | 9.43 | 14.48 | 16.08 | 72 | 49.13 | 0.20 | 81.58 | 25.38 | 15.15 | 29.05 |
| 5-14c | 48 | 193.30 | 0.03 | 26.81 | 7.93 | 15.42 | 15.12 | 72 | 50.87 | 0.21 | 81.23 | 22.16 | 17.90 | 30.68 |
| 5-15c | 48 | 171.98 | 0.16 | 34.46 | 11.32 | 15.07 | 16.75 | 72 | 24.63 | 0.28 | 93.81 | 28.43 | 17.32 | 29.58 |
| 5-16d | 48 | 182.46 | 0.35 | 28.49 | 10.66 | 15.72 | 14.77 | 72 | 46.30 | 0.95 | 77.46 | 27.99 | 17.59 | 25.71 |
| 5-16e | 48 | 171.82 | 0.17 | 30.99 | 11.78 | 17.81 | 16.21 | 72 | 50.29 | 0.37 | 78.74 | 27.69 | 20.41 | 21.31 |
| 5-17e | 48 | 186.94 | 0.10 | 30.46 | 9.52 | 12.31 | 14.91 | 72 | 45.82 | 0.19 | 86.08 | 26.35 | 13.47 | 26.22 |
| 5-21a | 48 | 177.76 | 0.18 | 31.71 | 10.47 | 15.87 | 16.27 | 72 | 39.80 | 0.42 | 85.25 | 26.79 | 18.17 | 26.32 |
| 5-21b | 48 | 177.01 | 0.44 | 29.60 | 11.39 | 13.95 | 16.59 | 72 | 41.38 | 1.24 | 78.78 | 27.99 | 14.95 | 28.67 |
| 5-26b | 48 | 174.31 | 0.15 | 31.39 | 8.99 | 14.76 | 16.97 | 72 | 57.27 | 0.25 | 78.38 | 22.73 | 16.81 | 23.61 |
| 5-26d | 48 | 175.31 | 0.08 | 34.93 | 11.69 | 13.45 | 15.39 | 72 | 44.33 | 0.20 | 88.86 | 27.63 | 15.56 | 23.21 |

The results of this example are consistent with the results of previous examples, demonstrating that strains expressing the X5PP enzymes of SEQ ID NOs: 202, 203, 204, 205, 206, 210, and 213 produce xylitol titers that are roughly equivalent to or greater than the xylitol titers produced by control strains expressing the X5PP of SEQ ID NO:200. As demonstrated in several examples above, while a strain is PCR verified to contain the desired polynucleotide sequence, said sequence may not be integrated at the correct loci, it may have had multiple copies of the sequence integrated into its genome, or a frameshift or other mutation caused an individual sister to vary from the others. The results here suggest that similar transformation occurrences are present in these sisters, but the results do demonstrate the effectiveness of the indicated X5PP enzymes.

### Example 26: Genetically Modified Moniliella pollinis Strains

UV mutagenesis (using a Hoefer UV Crosslinker at an energy of 360 uJ/cm³) and selection of strain 3-12d was used to generate a Moniliella pollinis strain with reduced foaming during shake flask fermentation. Strains with low-foaming phenotypes were selected based on visual evaluation of foaming in a shake flask fermentation compared to foaming on the parent 3-12d strain. The resulting low-foaming strain, containing two copies of an exogenous polynucleotide sequence encoding the XPDH of SEQ ID NO:14 integrated at the ER1 locus and one copy of a polynucleotide sequence encoding the RPE of SEQ ID NO: 180 integrated at the ER3 locus, was designated 6-1.

Strain 6-1 was transformed with the Cre recombinase plasmid of SEQ ID NO:288 using the transformation method outlined in Example 4. The resulting transformants were evaluated by colony PCR for removal of the G418 and zeocin resistance selection marker. A PCR verified isolate was designated strain 6-2.

Strain 6-2 was grown non-selectively on YPD plates to allow for the loss of the plasmid of SEQ ID NO:288. Biomass was struck for single colonies and evaluated by PCR to confirm loss of the plasmid. A PCR verified isolate was designated strain 6-3.

The indicated Moniliella pollinis parent strain, 6-3, was transformed with the bipartite transformation fragments as indicated in Table 34 using the transformation method outlined in Example 4. Resulting transformants were evaluated by colony PCR for integration of the indicated sequence. A PCR verified isolate was then designated as the indicated strain number in Table 34. In some instances, more than one PCR verified isolate, e.g., “sister” isolates, are indicated by letters following the strain number. For example, strain 6-4 has 5 sister isolates, strains 6-4a, 6-4b, 6-4c, 6-4d, and 6-4e.

For example, Strain 6-3 was transformed with SEQ ID NO:280 and SEQ ID NO:279. SEQ ID NO:279 contains, in order, a 3′ portion of a G418 resistance gene expression cassette (SEQ ID NO:172), an MpTEF1 terminator (SEQ ID NO:289), and a 3′ gpdIIB flanking sequence (SEQ ID NO:166). SEQ ID NO:280 contains a 5′ gpdIIB flanking sequence (SEQ ID NO:158), a PGK1 promoter (SEQ ID NO:135), a gene (SEQ ID NO:230) encoding the Moniliella pollinis phosphatase polypeptide of SEQ ID NO:210, a TDH3 terminator (SEQ ID NO:149), and a 5′ portion of a G418 resistance gene expression cassette (SEQ ID NO:175). Transformants were selected on PDA+G418 selection plates and incubated at 35 °C for at least 2 days until transformants grow. Resulting transformants were streaked for single colony isolation on PDA+G418 plates and single colonies were selected. Selected colonies were evaluated by colony PCR for integration of the indicated sequence. PCR verified isolates were designated strains 6-4a, 6-4b, 6-4c, 6-4d, and 6-4e.

The transformation cassettes of SEQ ID NOs: 281-287 for the phosphatase homologs outlined in Table 34 have the same components as SEQ ID NO:280 but included the indicated nucleotide sequence encoding the indicated polypeptide sequence. SEQ ID NO:181 was described in Example 12 and contained, in order, a 5′ ER3 flanking sequence (SEQ ID NO:155), an MpPYK1 promoter (SEQ ID NO:86), a gene encoding the M. pollinis RPE2 polypeptide of SEQ ID NO: 180, an MpPYK1 terminator (SEQ ID NO:146), and a 5′ portion of a zeocin resistance gene expression cassette (SEQ ID NO:169).

TABLE 34

| | | Phosphatase Homolog Expression Cassette | | | Second Bipartite |
|---|---|---|---|---|---|
| | | | | Encoded | Cassette |
| | Parent | Transformation | Nucleotide | Polypeptide | Transformation |
| Strain | Strain | SEQ ID NO: | SEQ ID NO: | SEQ ID NO: | SEQ ID NO: |
| 6-4a-e | 6-3 | 280 | 230 | 200 | 279 |
| 6-5a-e | 6-3 | 281 | 250 | 210 | 279 |
| 6-6a-e | 6-3 | 282 | 240 | 205 | 279 |
| 6-7a-e | 6-3 | 283 | 242 | 206 | 279 |
| 6-8a-e | 6-3 | 284 | 236 | 203 | 279 |
| 6-9a-e | 6-3 | 285 | 238 | 204 | 279 |
| 6-10a-e | 6-3 | 286 | 232 | 202 | 279 |
| 6-11a-e | 6-3 | 287 | 256 | 213 | 279 |
| 6-12a-c | 6-3 | 229 | 230 | 200 | 181 (encoding the RPE |
| | | | | | of SEQ ID NO: 180) |

Sequencing was carried out on strains 6-12a-c to determine at which sites the indicated polynucleotides integrated and what the final copy number of the given genes are. The parent strain 6-3 included 1 copy of a polynucleotide encoding the RPE of SEQ ID NO: 180 integrated at an ER3 loci on a single allele. In strain 6-12b, the second copy of the RPE encoding sequence integrated at the ER3 locus on the second allele resulting in a complete ER3 knockout. Strain 6-12b also was confirmed to have two copies of SEQ ID NO:230 encoding the X5PP enzyme of SEQ ID NO:200. However, in strains 6-12a and 6-12c, the transformation resulted in replacement of previously integrated sequence with new sequences encoding both the RPE of SEQ ID NO:180 and the X5PP of SEQ ID NO:200.

### Example 27: Shake Flask Fermentation Assay

Strains 6-3, 6-4a-e, 6-5a-e, 6-6a-e, 6-7a-e, 6-8a-e, 6-9a-e, 6-10a-e, and 6-11a-e were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

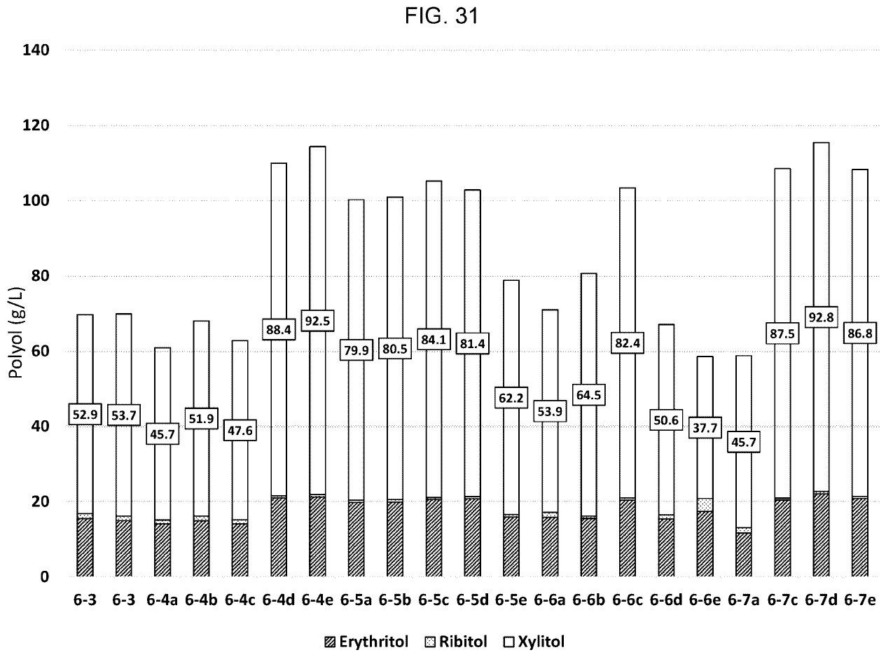

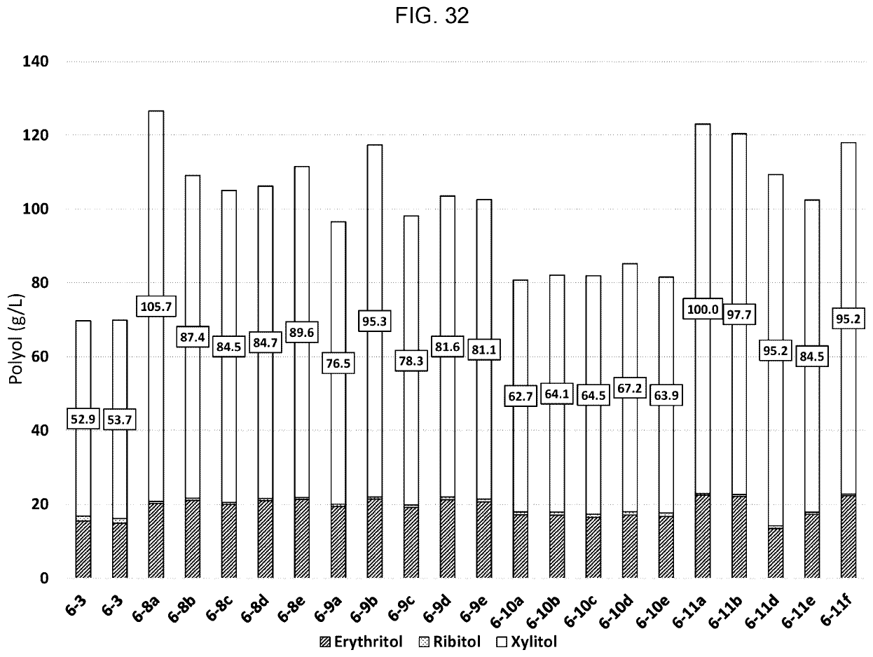

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Fermentation results for the 48- and 72-hour time points are reported in Table 35. Results from the 24- and 96-hour time points are not shown. Titers at the 24-hour time point are low, and after 96 hours, many of the cultures included low or no concentrations of glucose. Results of the 72-hour time point are also shown in FIGS. 31 and 32.

There was an error running the shake flask fermentation for strain 6-7b. While the results show little glucose consumption and low metabolite production, it's likely this was due to an error in the fermentation set up and not a defect of the strain.

TABLE 35
Shake Flask Results

| | Time | | | | | | | Time | | | | | | |
|---|---|---|---|---|---|---|---|---|---|---|---|---|---|---|
| | Point | Fermentation Broth Analyte (g/L) | | | | | | Point | Fermentation Broth Analyte (g/L) | | | | | |
| Strain | (h) | Glucose | Ribitol | Xylitol | Erythritol | Glycerol | Ethanol | (h) | Glucose | Ribitol | Xylitol | Erythritol | Glycerol | Ethanol |
| 6-3 | 48 | 175.23 | 0.47 | 16.20 | 7.98 | 33.73 | 12.42 | 72 | 74.55 | 1.26 | 52.90 | 15.52 | 41.60 | 16.32 |
| 6-3 | 48 | 177.95 | 0.46 | 16.79 | 7.51 | 31.23 | 13.19 | 72 | 79.43 | 1.26 | 53.69 | 14.93 | 37.63 | 17.15 |
| 6-4a | 48 | 188.89 | 0.38 | 12.57 | 7.47 | 30.95 | 11.48 | 72 | 89.37 | 1.12 | 45.73 | 14.04 | 39.24 | 17.53 |
| 6-4b | 48 | 178.93 | 0.44 | 15.57 | 7.57 | 31.32 | 13.00 | 72 | 81.86 | 1.23 | 51.91 | 14.91 | 37.98 | 17.21 |
| 6-4c | 48 | 186.85 | 0.41 | 13.77 | 7.35 | 29.99 | 12.78 | 72 | 87.75 | 1.16 | 47.60 | 14.04 | 36.84 | 18.35 |
| 6-4d | 48 | 156.42 | 0.22 | 26.66 | 8.66 | 28.48 | 18.50 | 72 | 12.98 | 0.60 | 88.37 | 20.99 | 29.42 | 30.35 |
| 6-4e | 48 | 141.80 | 0.24 | 31.78 | 9.81 | 29.01 | 20.73 | 72 | 6.76 | 0.64 | 92.47 | 21.24 | 28.75 | 30.70 |
| 6-5a | 48 | 165.73 | 0.21 | 23.21 | 8.05 | 30.12 | 16.74 | 72 | 29.77 | 0.61 | 79.90 | 19.77 | 32.55 | 26.59 |
| 6-5b | 48 | 162.71 | 0.23 | 23.90 | 8.27 | 30.54 | 17.15 | 72 | 27.62 | 0.61 | 80.46 | 19.88 | 32.77 | 27.29 |
| 6-5c | 48 | 155.78 | 0.24 | 26.92 | 8.78 | 30.42 | 18.46 | 72 | 21.02 | 0.62 | 84.10 | 20.49 | 31.47 | 27.53 |
| 6-5d | 48 | 162.15 | 0.23 | 23.91 | 8.22 | 31.23 | 16.51 | 72 | 23.79 | 0.62 | 81.45 | 20.73 | 34.14 | 25.02 |
| 6-5e | 48 | 180.59 | 0.24 | 17.12 | 7.70 | 30.33 | 15.09 | 72 | 63.64 | 0.66 | 62.22 | 15.89 | 33.73 | 25.24 |
| 6-6a | 48 | 173.32 | 0.46 | 17.11 | 7.89 | 32.77 | 14.11 | 72 | 74.88 | 1.26 | 53.90 | 15.83 | 39.17 | 17.89 |
| 6-6b | 48 | 169.78 | 0.25 | 21.31 | 7.85 | 30.55 | 14.43 | 72 | 61.77 | 0.64 | 64.46 | 15.54 | 35.43 | 20.92 |
| 6-6c | 48 | 155.19 | 0.24 | 26.38 | 8.69 | 30.22 | 18.35 | 72 | 23.76 | 0.65 | 82.40 | 20.37 | 31.34 | 26.97 |
| 6-6d | 48 | 181.48 | 0.42 | 15.26 | 7.83 | 31.08 | 12.93 | 72 | 81.21 | 1.02 | 50.58 | 15.44 | 37.13 | 18.03 |
| 6-6e | 48 | 199.31 | 0.72 | 10.16 | 7.55 | 26.98 | 11.89 | 72 | 89.35 | 3.48 | 37.70 | 17.34 | 34.03 | 22.16 |
| 6-7a | 48 | 208.23 | 0.45 | 13.22 | 5.75 | 19.47 | 12.39 | 72 | 105.11 | 1.38 | 45.71 | 11.68 | 25.07 | 22.57 |
| 6-7b | 48 | 302.65 | 0.05 | 0.94 | 1.01 | 1.70 | 0.29 | 72 | 302.46 | 0.06 | 1.18 | 1.35 | 2.11 | 0.72 |
| 6-7c | 48 | 155.96 | 0.22 | 26.92 | 8.76 | 29.44 | 18.83 | 72 | 15.66 | 0.58 | 87.50 | 20.39 | 30.65 | 29.75 |
| 6-7d | 48 | 150.90 | 0.24 | 27.85 | 9.44 | 31.53 | 18.68 | 72 | 9.09 | 0.62 | 92.75 | 22.06 | 33.04 | 25.39 |
| 6-7e | 48 | 162.89 | 0.23 | 24.74 | 8.55 | 30.44 | 17.71 | 72 | 16.13 | 0.59 | 86.82 | 20.82 | 32.02 | 28.46 |
| 6-8a | 48 | 146.28 | 0.20 | 34.48 | 9.36 | 26.31 | 21.79 | 72 | 0.55 | 0.59 | 105.72 | 20.25 | 22.71 | 31.74 |
| 6-8b | 48 | 153.44 | 0.22 | 28.22 | 9.00 | 29.83 | 18.19 | 72 | 16.50 | 0.59 | 87.43 | 21.12 | 30.88 | 27.85 |
| 6-8c | 48 | 158.99 | 0.22 | 26.90 | 8.66 | 26.81 | 18.64 | 72 | 27.35 | 0.56 | 84.49 | 19.97 | 27.24 | 27.51 |
| 6-8d | 48 | 163.54 | 0.22 | 26.12 | 8.74 | 28.30 | 18.18 | 72 | 22.74 | 0.53 | 84.66 | 21.04 | 29.53 | 26.82 |
| 6-8e | 48 | 153.49 | 0.20 | 29.21 | 9.10 | 28.68 | 19.27 | 72 | 14.24 | 0.53 | 89.59 | 21.34 | 29.36 | 27.68 |
| 6-9a | 48 | 165.15 | 0.26 | 22.59 | 8.14 | 30.86 | 16.83 | 72 | 29.86 | 0.67 | 76.48 | 19.38 | 33.40 | 27.51 |
| 6-9b | 48 | 151.98 | 0.22 | 29.41 | 9.19 | 28.92 | 19.04 | 72 | 5.67 | 0.61 | 95.29 | 21.42 | 29.28 | 30.46 |
| 6-9c | 48 | 166.26 | 0.25 | 23.23 | 7.89 | 28.93 | 17.39 | 72 | 31.14 | 0.65 | 78.30 | 19.17 | 30.87 | 27.90 |
| 6-9d | 48 | 156.94 | 0.27 | 25.10 | 8.37 | 30.48 | 17.14 | 72 | 20.92 | 0.71 | 81.56 | 21.29 | 31.87 | 26.50 |
| 6-9e | 48 | 159.38 | 0.28 | 24.66 | 8.57 | 31.47 | 17.29 | 72 | 24.55 | 0.70 | 81.08 | 20.73 | 33.39 | 25.00 |
| 6-10a | 48 | 172.26 | 0.31 | 18.48 | 7.66 | 32.34 | 15.04 | 72 | 52.38 | 0.82 | 62.75 | 17.17 | 36.74 | 22.21 |
| 6-10b | 48 | 169.87 | 0.33 | 20.07 | 7.73 | 30.29 | 15.88 | 72 | 54.25 | 0.87 | 64.11 | 17.09 | 33.53 | 24.03 |
| 6-10c | 48 | 167.47 | 0.34 | 20.16 | 7.85 | 31.12 | 16.19 | 72 | 51.40 | 0.88 | 64.53 | 16.47 | 34.68 | 24.67 |
| 6-10d | 48 | 167.97 | 0.34 | 20.56 | 7.73 | 30.54 | 16.61 | 72 | 47.66 | 0.89 | 67.16 | 17.13 | 34.06 | 24.31 |
| 6-10e | 48 | 169.40 | 0.37 | 20.08 | 7.63 | 30.71 | 16.17 | 72 | 51.73 | 0.93 | 63.85 | 16.75 | 34.87 | 22.46 |
| 6-11a | 48 | 150.19 | 0.18 | 31.53 | 9.42 | 27.23 | 19.68 | 72 | 4.84 | 0.46 | 99.96 | 22.54 | 26.04 | 29.12 |
| 6-11b | 48 | 156.06 | 0.17 | 29.65 | 9.01 | 27.28 | 18.59 | 72 | 8.21 | 0.46 | 97.71 | 22.19 | 25.88 | 28.62 |
| 6-11d | 48 | 158.15 | 0.26 | 30.78 | 5.86 | 24.70 | 20.42 | 72 | 17.46 | 0.76 | 95.15 | 13.42 | 22.86 | 34.12 |
| 6-11e | 48 | 155.89 | 0.25 | 28.08 | 7.65 | 30.46 | 18.12 | 72 | 28.49 | 0.62 | 84.51 | 17.27 | 28.53 | 25.44 |
| 6-11f | 48 | 156.20 | 0.18 | 28.82 | 8.77 | 26.93 | 18.48 | 72 | 8.56 | 0.44 | 95.22 | 22.31 | 26.27 | 28.95 |

The results demonstrate that the same increase in xylitol titer and yield can be achieved when the X5PP encoding sequence is integrated at the gpdIIB locus as was seen in previous examples where the sequence was integrated at the ER3 locus. As demonstrated in several examples above, while a strain is PCR verified to contain the desired polynucleotide sequence, said sequence may not be integrated at the correct loci, it may have had multiple copies of the sequence integrated into its genome, or a frameshift or other mutation caused an individual sister to vary from the others. The results here suggest that similar transformation occurrences are present in these sisters, but the results do demonstrate the effectiveness of the indicated X5PP enzymes.

### Example 28: Shake Flask Fermentation Assay

Strains 6-3, 6-12a, 6-12b, 6-12c, 6-4d, 6-4e, 6-7d, 6-8a, 6-8e, 6-9b, 6-9d, 6-11a, 6-11b, 6-11c, 6-11d, 6-Ile were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

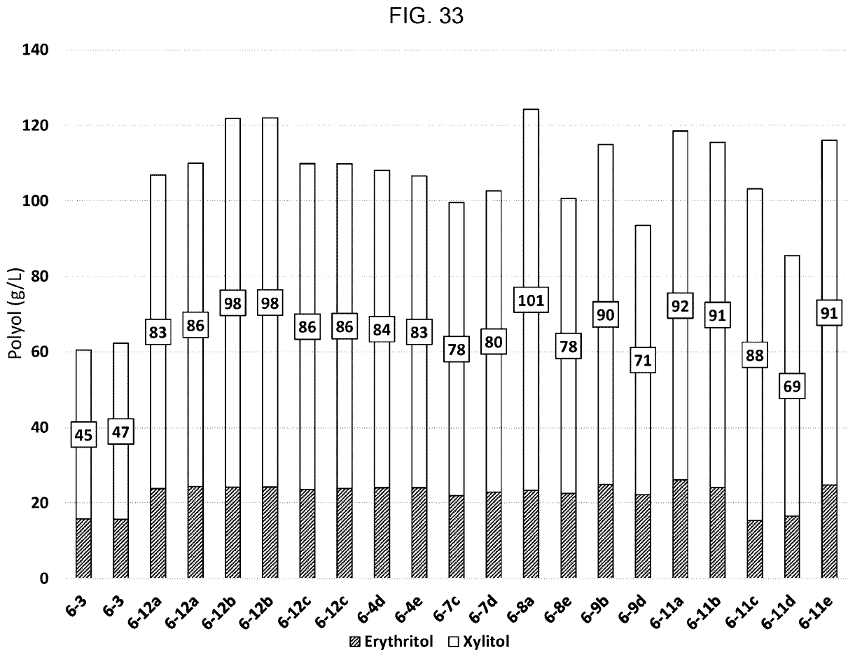

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Fermentation results for the 48- and 72-hour time points are reported in Table 36. Results from the 24- and 96-hour time points are not shown. Titers at the 24-hour time point are low, and after 96 hours, many of the cultures included low or no concentrations of glucose. Results for the 72-hour time point are also shown in FIG. 33.

There was an error running the shake flask fermentation for strain 6-7b. While the results show little glucose consumption and low metabolite production, it's likely this was due to an error in the fermentation set up and not a defect of the strain.

TABLE 36
Shake Flask Results

| | Time | | | | | | | Time | | | | | | |
|---|---|---|---|---|---|---|---|---|---|---|---|---|---|---|
| | Point | Fermentation Broth Analyte (g/L) | | | | | | Point | Fermentation Broth Analyte (g/L) | | | | | |
| Strain | (h) | Glucose | Ribitol | Xylitol | Erythritol | Glycerol | Ethanol | (h) | Glucose | Ribitol | Xylitol | Erythritol | Glycerol | Ethanol |
| 6-3 | 48 | 197.69 | 0.30 | 11.18 | 8.03 | 36.82 | 9.55 | 72 | 81.79 | 0.97 | 44.76 | 15.75 | 47.47 | 16.61 |
| 6-3 | 48 | 195.47 | 0.30 | 12.58 | 7.78 | 34.93 | 10.64 | 72 | 84.66 | 1.09 | 46.62 | 15.65 | 45.11 | 16.09 |
| 6-12a | 48 | 159.28 | 0.14 | 25.14 | 9.55 | 31.86 | 17.84 | 72 | 15.94 | 0.48 | 83.04 | 23.80 | 32.90 | 29.20 |
| 6-12a | 48 | 155.72 | 0.17 | 26.15 | 9.76 | 31.35 | 18.40 | 72 | 14.08 | 0.47 | 85.64 | 24.35 | 32.17 | 28.77 |
| 6-12b | 48 | 164.72 | 0.06 | 28.64 | 9.00 | 20.60 | 19.01 | 72 | 3.17 | 0.29 | 97.69 | 24.14 | 20.26 | 33.06 |
| 6-12b | 48 | 169.47 | 0.06 | 28.72 | 8.87 | 20.47 | 19.15 | 72 | 3.50 | 0.29 | 97.80 | 24.19 | 20.54 | 33.33 |
| 6-12c | 48 | 157.09 | 0.12 | 25.84 | 9.51 | 30.20 | 19.27 | 72 | 10.00 | 0.43 | 86.30 | 23.57 | 31.01 | 30.99 |
| 6-12c | 48 | 154.67 | 0.14 | 26.64 | 9.79 | 31.58 | 19.03 | 72 | 9.00 | 0.41 | 86.01 | 23.85 | 31.96 | 30.44 |
| 6-4d | 48 | 159.31 | 0.12 | 25.21 | 9.56 | 31.73 | 17.73 | 72 | 14.86 | 0.41 | 84.01 | 24.04 | 32.60 | 29.53 |
| 6-4e | 48 | 159.98 | 0.14 | 25.08 | 9.37 | 30.91 | 17.65 | 72 | 17.01 | 0.42 | 82.58 | 24.05 | 31.79 | 29.59 |
| 6-7c | 48 | 167.95 | 0.16 | 21.80 | 8.87 | 33.16 | 16.27 | 72 | 24.99 | 0.41 | 77.60 | 21.97 | 35.64 | 28.35 |
| 6-7d | 48 | 163.65 | 0.11 | 23.12 | 9.30 | 34.13 | 16.86 | 72 | 21.64 | 0.36 | 79.71 | 22.91 | 37.12 | 26.70 |
| 6-8a | 48 | 148.89 | 0.15 | 31.89 | 10.16 | 29.85 | 20.96 | 72 | 1.19 | 0.35 | 100.88 | 23.38 | 26.53 | 30.38 |
| 6-8e | 48 | 163.10 | 0.14 | 23.96 | 9.29 | 32.22 | 17.20 | 72 | 26.49 | 0.43 | 78.12 | 22.54 | 34.30 | 28.18 |
| 6-9b | 48 | 152.17 | 0.14 | 27.88 | 10.25 | 32.51 | 18.58 | 72 | 5.97 | 0.45 | 89.89 | 24.99 | 33.21 | 30.05 |
| 6-9d | 48 | 170.93 | 0.21 | 19.04 | 8.97 | 36.04 | 14.22 | 72 | 30.09 | 0.59 | 71.28 | 22.22 | 39.93 | 26.88 |
| 6-11a | 48 | 156.65 | 0.09 | 27.73 | 10.14 | 32.05 | 17.29 | 72 | 10.45 | 0.39 | 92.39 | 26.11 | 32.84 | 25.41 |
| 6-11b | 48 | 154.72 | 0.16 | 27.90 | 10.17 | 32.22 | 17.90 | 72 | 10.48 | 0.25 | 91.40 | 24.08 | 32.95 | 28.13 |
| 6-11c | 48 | 159.24 | 0.16 | 29.48 | 6.56 | 32.51 | 17.54 | 72 | 26.93 | 0.55 | 87.79 | 15.37 | 31.11 | 26.67 |
| 6-11d | 48 | 159.45 | 0.20 | 25.80 | 7.69 | 31.73 | 17.25 | 72 | 58.50 | 0.37 | 68.86 | 16.57 | 31.86 | 22.87 |
| 6-11e | 48 | 155.63 | 0.12 | 26.95 | 10.01 | 32.00 | 18.09 | 72 | 10.18 | 0.41 | 91.29 | 24.75 | 32.95 | 26.24 |

### Example 29: Erythrose Reductase Candidate In Vitro Enzyme Assays

Polynucleotides encoding suspected erythrose reductase (ER) enzymes (Table 37) were cloned into a vector containing a T7 promoter and terminator for cell-free protein expression using purified recombinant T7 RNA polymerase for transcription and purified recombinant ribosome for translation (New England Biolabs, PURExpress® In Vitro Protein Synthesis). Enzyme expression using the in vitro system was confirmed by gel electrophoresis (results not shown)

TABLE 37

| | | Gene Sequence | Gene Sequence | Amino Acid |
|---|---|---|---|---|
| | | (with introns) | (without introns) | Sequence |
| Source Organism | Gene Name | SEQ ID NO: | SEQ ID NO: | SEQ ID NO: |
| Moniliella pollinis | RCSR18717 | 290 | 291 | 292 |
| Moniliella pollinis | RCSR26640 | 293 | 294 | 295 |
| Moniliella pollinis | RCSR11551 | 296 | 297 | 298 |
| Moniliella pollinis | RCSR18367 (“ER1”) | 306 | 307 | 308 |
| Moniliella pollinis | RCSR08378 (“ER3”) | 309 | 310 | 311 |

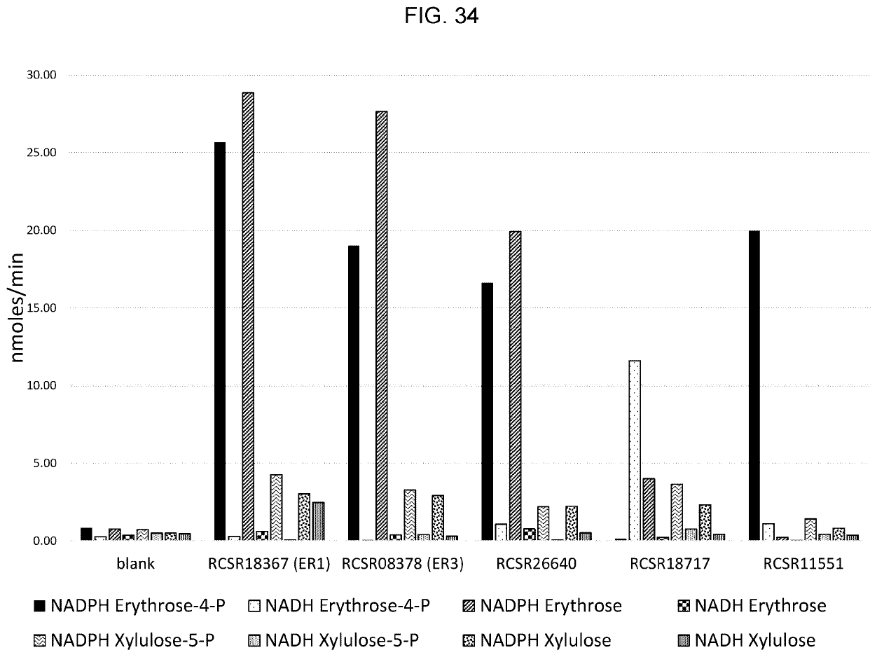

Cell-free synthesized proteins were analyzed for activity on four substrates (erythrose, erythrose-4-phosphate, xylulose, and xylulose-5-phosphase) with either NADPH or NADH cofactors. Enzyme assays were carried out in 96-well plates with 180 μL of reaction mixture as outlined in Table 38 and 20 μL of the in vitro enzyme expression product. Reaction progress over 15 minutes was monitored by the NAD (P) H concentration as measured by light absorbance at 340 nm. Results are shown in Table 39 and FIG. 34.

TABLE 38

| | final |
|---|---|
| | conc |
| Component | (mM) |
| HEPES, pH 8 | 50 |
| ZnSO4 | 0.001 |
| NADPH -OR- | 0.25 |
| NADH | |
| Erythrose-4-P -OR- | 3 |
| Erythrose -OR- | |
| Xylulose-5-P -OR- | |
| Xylulose | |
| water | to 180 uL |

TABLE 39

| | Enzyme Activity (nmoles/min) | | | | | | | |
|---|---|---|---|---|---|---|---|---|
| | NADPH cofactor | | | | NADH cofactor | | | |
| ER | erythrose-4- | | xylulose-5- | | erythrose-4- | | xylulose-5- | |
| Candidate | phosphate | erythrose | phosphate | xylulose | phosphate | erythrose | phosphate | xylulose |
| blank | 0.84 | 0.77 | 0.71 | 0.50 | 0.28 | 0.37 | 0.49 | 0.46 |
| RCSR18717 | 0.16 | 4.01 | 3.64 | 2.32 | 11.61 | 0.23 | 0.76 | 0.43 |
| RCSR26640 | 16.62 | 19.92 | 2.21 | 2.24 | 1.08 | 0.77 | 0.05 | 0.52 |
| RCSR11551 | 19.97 | 0.23 | 1.41 | 0.82 | 1.11 | 0.03 | 0.43 | 0.38 |
| RCSR18367 | 25.68 | 28.86 | 4.26 | 3.04 | 0.30 | 0.61 | 0.07 | 2.47 |
| “ER1” | | | | | | | | |
| RCSR08378 | 19.00 | 27.65 | 3.29 | 2.92 | 0.01 | 0.39 | 0.41 | 0.30 |
| “ER3” | | | | | | | | |

While ER1 and ER3 were predicted to work on the substrate combination of NADPH and erythrose, the assay demonstrated that they are also active on the substrate combination of NADPH and erythrose-4-phosphate. Additionally, The RSCR18717, RCSR26440, and RCSR11551 ER enzymes also had activity on the tested substrates. RCSR26640 had activity on the same substrates as ER1 and ER3, while RCSR11551 only acted on the NADPH and erythrose-4-phosphate substrate combination. RSR18717 was the only enzyme to show activity with the NADH cofactor with erythrose-4-phosphate. Additionally, none of the enzymes tested showed activity on the xylitol pathway intermediates.

### Example 30: Genetically Modified Moniliella pollinis Strains

The strains described in this example were developed to test in vivo reduction of erythritol production through knockouts of the candidate ER genes and/or reduced expression of the candidate ER enzymes. A summary of the engineered strains is provided in Table 40.

Strains 7-1a-b

Strain 6-12b was transformed with SEQ ID NO:299 using the transformation protocol outlined in Example 4 and positive transformants were selected using nourseothricin selection plates. SEQ ID NO:299 contains a deletion construct to remove one copy of the RSCR18717 gene. Two PCR verified sister isolates, in which one copy of the RCSR18717 was knocked out, were designated strains 7-1a and 7-1b.

Strain 7-2a-d

Strain 7-1a was transformed with SEQ ID NO:300 using the transformation protocol outlined in Example 4. SEQ ID NO:300 contains a construct to loop out the zeocin and nourseothricin resistance selection markers. Two PCR verified sister isolates, in which the zeocin and nourseothricin resistance selection markers were removed, where designated strains 7-2a and 7-2b.

Strain 7-1b was transformed with SEQ ID NO:300 using the transformation protocol outlined in Example 4. SEQ ID NO:300 contains a construct to loop out the zeocin and nourseothricin resistance selection markers. Two PCR verified sister isolates, in which the zeocin and nourseothricin resistance selection markers were removed, were designated strains 7-2c and 7-2d.

Strains 7-3a-b

Strain 7-2c was transformed with SEQ ID NO:301 using the transformation protocol outlined in Example 4 and positive transformants were selected using geneticin (G418) selection plates. SEQ ID NO:301 contains a deletion construct to remove the second copy (i.e., only remaining copy) of the RCSR18717 gene. Two PCR verified sister isolates, in which both copies of the RCSR18717 gene were knocked out, were designated strains 7-3a and 7-3b.

Strains 7-4a-b

Strain 6-12b was transformed with SEQ ID NO:302 using the transformation protocol outlined in Example 4 and positive transformants were selected using nourseothricin selection plates. SEQ ID NO:302 contained a deletion construct to remove one copy of the RCSR26640 gene. Two PCR verified sister isolates, in which one copy of the RCSR26640 gene was knocked out, were designated strains 7-4a and 7-4b.

Strains 7-5a-d

Strain 7-4a was transformed with SEQ ID NO:300 using the transformation protocol outlined in Example 4. SEQ ID NO:300 contains a construct to loop out the zeocin and nourseothricin resistance selection markers. Two PCR verified sister isolates, in which the zeocin and nourseothricin resistance selection markers were removed, were designated strains 7-5a and 7-5b.

Strain 7-4b was transformed with SEQ ID NO:300 using the transformation protocol outlined in Example 4. SEQ ID NO:300 contains a construct to loop out the zeocin and nourseothricin resistance selection markers. Two PCR verified sister isolates, in which the zeocin and nourseothricin resistance selection markers were removed, were designated strains 7-5c and 7-5d.

Strain 7-6a-c

Strain 7-5c was transformed with SEQ ID NO:302 using the transformation protocol outlined in Example 4 and positive transformants were selected on nourseothricin selection plates. Three PCR verified sister isolate, in which both copies of the RCSR26640 gene were knocked out, were designated strains 7-6a, 7-6b, and 7-6c.

Strain 7-7a-b

Strain 6-12b was transformed with SEQ ID NO:303 using the transformation protocol outlined in Example 4 and positive transformants were selected using nourseothricin selection plates. SEQ ID NO:303 contained a deletion construct to remove the single copy of the RCSR11551 gene. Two PCR verified sister isolates, in which the single copy of the RCSR11551 gene was knocked out, were designated strains 7-7a and 7-7b.

### Strain 7-8

Strain 7-5c was transformed with SEQ ID NO:304 using the transformation protocol outlined in Example 4 and positive transformants were selected on geneticin (G418) selection plates. SEQ ID NO:304 contained a RCSR26440 5′ flanking sequence, a gene encoding the L. rhamnosus XPDH of SEQ ID NO: 14, an Mp6PGD terminator (SEQ ID NO: 140), a polynucleotide sequence encoding the G418 resistance selection markers, and a RCSR26440 3′ flanking sequence. A PCR verified isolate, in which one copy of the RCSR26640 gene has been replaced with a gene encoding the XPDH of SEQ ID NO:14, was designated train 7-8.

Strain 7-14a-e

Strain 6-12b was transformed with SEQ ID NO:305 and SEQ ID NO:279 using the transformation protocol outlined in Example 4. SEQ ID NO:305 contains a 5′ gpdIIB flanking sequence, an MpPGK1 promoter, a copy of the RCSR18717 gene of SEQ ID NO:290, an MpTDH3 terminator, and a 5′ portion of the G418 resistance gene. SEQ ID NO:279 contains a 3′ fragment of the G418 resistance gene, an MpTEF1 terminator, and a 3′ gpdIIB flanking sequence. PCR verified isolates were designated strains 7-14a, 7-14b, 7-14c, 7-14d, and 7-14e.

TABLE 40

| | | Transformation | |
|---|---|---|---|
| | Parent | fragment | |
| Strain | Strain | SEQ ID NO: | Notes |
| 7-1a-b | 6-12b | 299 | Knock out of 1 allele of the RCSR18717 gene |
| 7-2a-b | 7-1a | 300 | Loop out of NAT and zeocin resistance markers |
| 7-2c-d | 7-1b | | |
| 7-3a-b | 7-2c | 301 | Knock out of both alleles of the RCSR18717 gene |
| 7-4a-b | 6-12b | 302 | Knock out of 1 allele of the RCSR26640 gene |
| 7-5a-b | 7-4a | 300 | Loop out of NAT and zeocin resistance markers |
| 7-5c-d | 7-4b | | |
| 7-6a-c | 7-5c | 302 | Knock out of both alleles of the RCSR26640 gene |
| 7-7a-b | 6-12b | 303 | Knock out of RCSR11551 gene (gene is haploid, |
| | | | so this is a complete knock out) |
| 7-8 | 7-5c | 304 | One copy of RCSR26640 replaced with a gene |
| | | | encoding the XPDH of SEQ ID NO: 14. |
| 7-14a-e | 6-12b | 305 + 279 | Overexpression of RCSR18717 by addition of |
| | | | RCSR18717 gene at the gpdIIB locus |

Strains 7-9 through 7-13

The indicated Moniliella pollinis parent strain in Table 41 was transformed with the bipartite transformation fragments as indicated in Table 41 using the transformation method outlined in Example 4. Resulting transformants were evaluated by colony PCR for integration of the indicated sequence. A PCR verified isolate was then designated as the indicated strain number in Table 41. In some instances, more than one PCR verified isolate, e.g., “sister” isolates, are indicated by letters following the strain number. For example, strain 7-12 has 5 sister isolates, strains 7-12a, 7-12b, 7-12c, 7-12d, and 7-12e.

For example, Strain 7-2a was transformed with SEQ ID NO:280 and SEQ ID NO:279. SEQ ID NO:279 contains, in order, a 3′ portion of a G418 resistance gene expression cassette (SEQ ID NO:172), an MpTEF1 terminator (SEQ ID NO:289), and a 3′ gpdIIB flanking sequence (SEQ ID NO:166). SEQ ID NO:280 contains a 5′ gpdIIB flanking sequence (SEQ ID NO:158), a PGK1 promoter (SEQ ID NO:135), a gene (SEQ ID NO:230) encoding the Moniliella pollinis phosphatase polypeptide of SEQ ID NO:210, a TDH3 terminator (SEQ ID NO:149), and a 5′ portion of a G418 resistance gene expression cassette (SEQ ID NO:175). Transformants were selected on PDA+G418 selection plates and incubated at 35 °C for at least 2 days until transformants grow. Resulting transformants were streaked for single colony isolation on PDA+G418 plates and single colonies were selected. Selected colonies were evaluated by colony PCR for integration of the indicated sequence. PCR verified isolates were designated strains 7-9a, 7-9b, 7-9c, and 7-9d.

SEQ ID NO:312 contains a 5′ gpdIIB flanking sequence (SEQ ID NO:158), an MpPYK1 promoter (SEQ ID NO:86), a polynucleotide encoding the M. pollinis X5PP enzyme of SEQ ID NO:200, an MpENO1 terminator (SEQ ID NO:142), an MpPGK1 promoter (SEQ ID NO: 135), a polynucleotide sequence encoding the M. pollinis X5PP enzyme of SEQ ID NO:200, an MpTDH3 terminator (SEQ ID NO:149), and a 5′ portion of a G418 resistance marker (SEQ ID NO: 175. SEQ ID NO:313 contains a 3′ portion of a G418 resistance marker (SEQ ID NO:172), an MpENO1 promoter (SEQ ID NO:136), the gene sequence of SEQ ID NO:100 encoding the L. rhamnosus XPDH of SEQ ID NO:14, an MpPYK1 terminator (SEQ ID NO:146), an MpTDH3 promoter (SEQ ID NO:132), a polynucleotide sequence encoding the RPE enzyme of SEQ ID NO: 180, an Mp6PGD terminator (SEQ ID NO:140), an MpPYK1 promoter (SEQ ID NO:86), a polynucleotide encoding the M. pollinis X5PP of SEQ ID NO:200, an MpENO1 terminator (SEQ ID NO: 142), and a 3′ gpdIIB flanking sequence (SEQ ID NO: 166). SEQ ID NO:314 contains a 5′ gpdIIB flanking sequence (SEQ ID NO:158), an MpTDH3 promoter (SEQ ID NO:132), the polynucleotide of SEQ ID NO:100 encoding the L. rhamnosus XPDH of SEQ ID NO: 14, an MpPYK1 terminator (SEQ ID NO:146), an MpPGK1 promoter (SEQ ID NO:135), a polynucleotide encoding the RPE enzyme of SEQ ID NO: 180, an MpTDH3 terminator (SEQ ID NO: 149), an MpPYK1 promoter (SEQ ID NO:86), a polynucleotide encoding the M. pollinis X5PP enzyme of SEQ ID NO:200, an MpENO1 terminator (SEQ ID NO:142), and a 5′ fragment of a G418 resistance marker (SEQ ID NO:175).

TABLE 41

| | | First Bipartite Cassette | | Second Bipartite Cassette | |
|---|---|---|---|---|---|
| | | Transfor- | Encoded | Transfor- | Encoded |
| | Parent | mation | Polypeptide | mation | Polypeptide |
| Strain | Strain | SEQ ID NO: | SEQ ID NO: | SEQ ID NO: | SEQ ID NO: |
| 7-9a-d | 7-2a | 280 | 200 | 279 | — |
| 7-10 | 7-2c | 280 | 200 | 279 | — |
| 7-11a-d | 7-2c | 312 | 2x 200 | 279 | — |
| 7-12a-e | 7-2c | 312 | 2x 200 | 313 | 14, 180, 200 |
| 7-13a-b | 7-2c | 314 | 14, 180, 200 | 313 | 14, 180, 200 |

### Example 31: Shake Flask Fermentation Assay

Strains 6-12b, 7-4a, 7-4b, 7-1a, 7-1b, 7-7a, and 7-7b were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

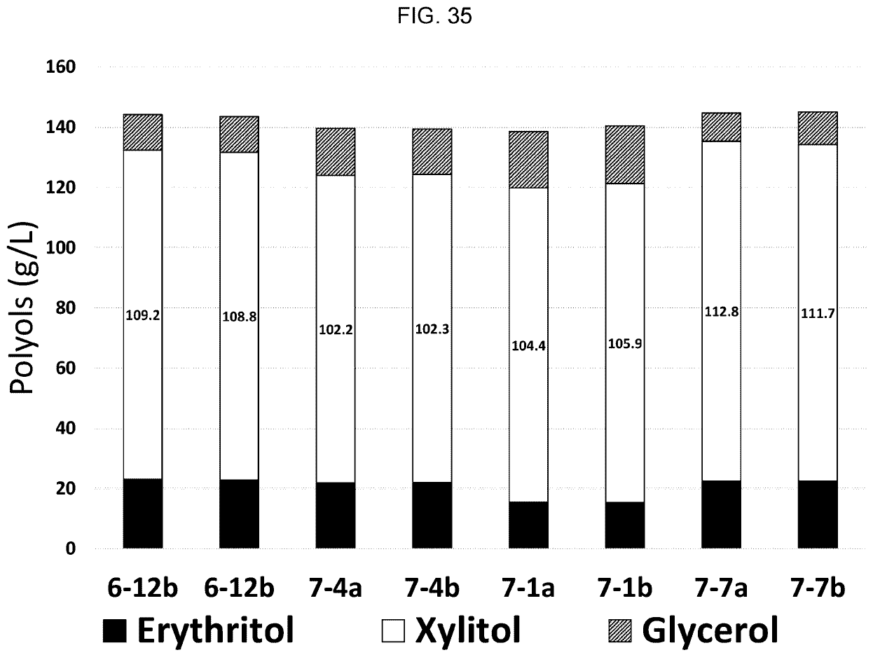

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Results are shown in Tables 42 and 43 and results for the 72-hour time point are also shown in FIG. 35.

Overall, strains 7-1a and 7-1b showed the greatest reduction in erythritol production (32%) relative to the parent (6-12b). However, this strain also showed a decrease in xylitol production (7.1%) and an increase in glycerol production (35%).

TABLE 42

| | Time | | | | | | | Time | | | | | | |
|---|---|---|---|---|---|---|---|---|---|---|---|---|---|---|
| | Point | Fermentation Broth Analyte (g/L) | | | | | | Point | Fermentation Broth Analyte (g/L) | | | | | |
| Strain | (h) | Glucose | Ribitol | Xylitol | Erythritol | Glycerol | Ethanol | (h) | Glucose | Ribitol | Xylitol | Erythritol | Glycerol | Ethanol |
| 6-12b | 24 | 276.62 | 0.03 | 1.55 | 1.06 | 4.32 | 0.13 | 48 | 168.65 | 0.14 | 26.77 | 7.78 | 21.27 | 16.89 |
| 6-12b | 24 | 279.28 | 0.00 | 1.58 | 1.07 | 4.56 | 0.27 | 48 | 167.61 | 0.13 | 26.71 | 7.73 | 20.96 | 16.70 |
| 7-4a | 24 | 279.49 | 0.03 | 1.42 | 0.93 | 3.79 | 0.24 | 48 | 170.31 | 0.11 | 25.84 | 7.33 | 19.58 | 16.73 |
| 7-4b | 24 | 281.54 | 0.04 | 1.43 | 0.95 | 3.72 | 0.21 | 48 | 171.64 | 0.12 | 25.91 | 7.24 | 19.16 | 16.92 |
| 7-1a | 24 | 281.47 | 0.04 | 1.39 | 0.57 | 3.76 | 0.26 | 48 | 194.37 | 0.10 | 18.89 | 4.26 | 23.00 | 11.75 |
| 7-1b | 24 | 282.38 | 0.05 | 1.51 | 0.66 | 4.17 | 0.27 | 48 | 182.43 | 0.11 | 22.31 | 4.88 | 23.50 | 13.44 |
| 7-7a | 24 | 278.83 | 0.01 | 1.51 | 1.05 | 4.59 | 0.28 | 48 | 173.82 | 0.12 | 25.90 | 7.07 | 19.56 | 17.24 |
| 7-7b | 24 | 283.19 | 0.06 | 1.56 | 0.99 | 4.08 | 0.34 | 48 | 182.43 | 0.11 | 22.15 | 6.36 | 20.95 | 14.68 |
| 6-12b | 72 | 0.26 | 0.35 | 101.70 | 22.48 | 18.11 | 29.13 | 96 | 0.02 | 0.40 | 109.24 | 23.11 | 11.82 | 16.95 |
| 6-12b | 72 | 0.45 | 0.35 | 102.00 | 22.30 | 18.23 | 29.91 | 96 | 0.02 | 0.40 | 108.83 | 22.82 | 11.91 | 19.91 |
| 7-4a | 72 | 2.35 | 0.33 | 99.01 | 21.90 | 16.78 | 31.36 | 96 | 0.58 | 0.33 | 102.18 | 21.84 | 15.61 | 22.72 |
| 7-4b | 72 | 2.44 | 0.33 | 98.43 | 21.85 | 16.15 | 31.34 | 96 | 0.55 | 0.33 | 102.34 | 22.00 | 15.05 | 22.11 |
| 7-1a | 72 | 11.45 | 0.32 | 90.94 | 15.29 | 24.88 | 32.68 | 96 | 0.02 | 0.38 | 104.40 | 15.44 | 18.71 | 23.48 |
| 7-1b | 72 | 4.15 | 0.34 | 97.14 | 15.12 | 24.03 | 32.32 | 96 | 0.02 | 0.38 | 105.87 | 15.41 | 19.11 | 23.08 |
| 7-7a | 72 | 1.42 | 0.35 | 101.33 | 22.17 | 17.97 | 31.60 | 96 | 0.02 | 0.42 | 112.84 | 22.49 | 9.36 | 19.79 |
| 7-7b | 72 | 0.26 | 0.35 | 101.70 | 22.48 | 18.11 | 29.13 | 96 | 0.01 | 0.44 | 111.70 | 22.53 | 10.82 | 19.67 |

TABLE 43

| Strain | 6-12b | 6-12b | 7-4a | 7-4b | 7-1a | 7-1b | 7-7a | 7-7b |
|---|---|---|---|---|---|---|---|---|
| Xylitol | 36.5 | 36.4 | 34.2 | 34.3 | 34.9 | 35.4 | 37.7 | 37.4 |
| Yield (%) | | | | | | | | |

### Example 32: Shake Flask Fermentation Assay

Strains 6-12b, 7-1a, 7-1b, 7-2a, 7-2b, 7-2c, 7-2d, 7-4a, 7-4b, 7-5a, 7-5b, 7-5c, and 7-5d were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

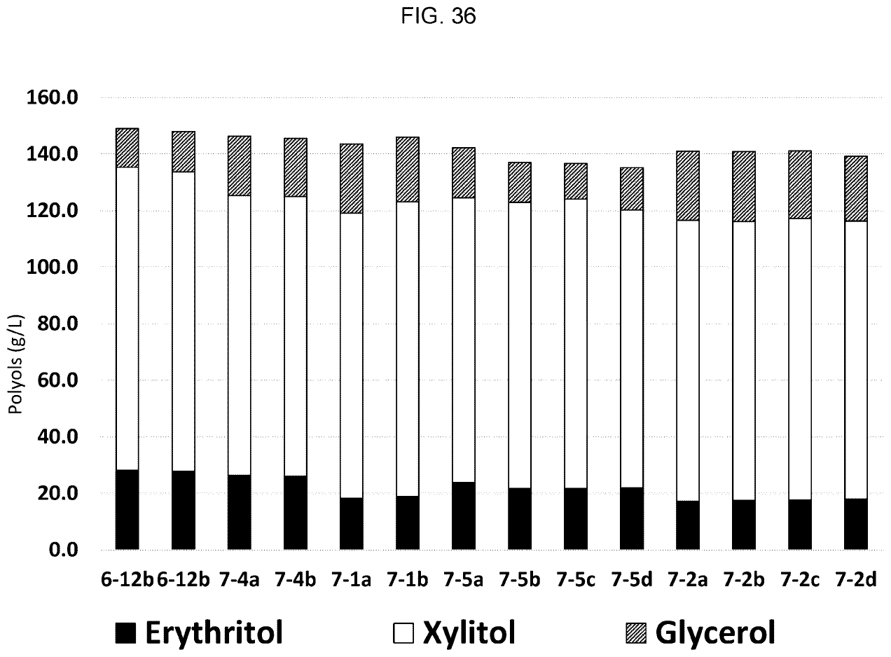

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Results are shown in Table 44 and results for the 96-hour time point are also shown in FIG. 36.

Overall, strains 7-2a-d showed a decrease in erythritol relative to the grandparent strain 6-12b. The erythritol production in strains 7-2a-d is similar to strains 7-1a-b, and all six of these strains have a knockout of one copy of the RCSR18717 gene. Strains 7-5a-d also show a slight decrease in erythritol production relative to the grandparent strain 6-b and this decrease is greater than the decrease seen in parent strains 7-4a-b.

TABLE 44

| | Time | | | | | | | Time | | | | | | |
|---|---|---|---|---|---|---|---|---|---|---|---|---|---|---|
| | Point | Fermentation Broth Analyte (g/L) | | | | | | Point | Fermentation Broth Analyte (g/L) | | | | | |
| Strain | (h) | Glucose | Ribitol | Xylitol | Erythritol | Glycerol | Ethanol | (h) | Glucose | Ribitol | Xylitol | Erythritol | Glycerol | Ethanol |
| 6-12b | 24 | 284.49 | 0.00 | 1.60 | 1.38 | 5.94 | 0.00 | 48 | 157.02 | 0.12 | 29.74 | 10.52 | 22.33 | 18.09 |
| 6-12b | 24 | 285.71 | 0.00 | 1.55 | 1.26 | 5.80 | 0.00 | 48 | 162.85 | 0.10 | 28.90 | 10.27 | 22.07 | 18.29 |
| 7-4a | 24 | 286.64 | 0.00 | 1.75 | 1.25 | 5.39 | 0.37 | 48 | 168.67 | 0.09 | 25.91 | 9.10 | 22.01 | 16.74 |
| 7-4b | 24 | 284.64 | 0.00 | 1.58 | 1.19 | 5.19 | 0.41 | 48 | 167.70 | 0.11 | 25.36 | 9.09 | 21.91 | 16.48 |
| 7-1a | 24 | 286.73 | 0.00 | 1.51 | 0.63 | 5.08 | 0.31 | 48 | 190.79 | 0.07 | 19.77 | 5.48 | 25.33 | 12.07 |
| 7-1b | 24 | 287.92 | 0.00 | 1.61 | 0.83 | 5.70 | 0.00 | 48 | 179.74 | 0.07 | 23.34 | 6.39 | 25.08 | 14.47 |
| 7-5a | 24 | 286.22 | 0.00 | 1.37 | 1.43 | 5.97 | 0.37 | 48 | 162.71 | 0.13 | 26.15 | 8.56 | 19.62 | 20.20 |
| 7-5b | 24 | 285.35 | 0.00 | 1.60 | 1.38 | 5.01 | 0.60 | 48 | 155.07 | 0.19 | 29.88 | 9.45 | 16.30 | 23.33 |
| 7-5c | 24 | 283.53 | 0.00 | 1.55 | 1.27 | 5.00 | 0.65 | 48 | 155.34 | 0.16 | 29.76 | 9.04 | 17.07 | 21.92 |
| 7-5d | 24 | 282.01 | 0.00 | 1.48 | 1.51 | 6.32 | 0.78 | 48 | 144.68 | 0.18 | 30.68 | 10.11 | 17.89 | 24.10 |
| 7-2a | 24 | 284.74 | 0.00 | 1.33 | 1.01 | 6.66 | 0.50 | 48 | 168.80 | 0.16 | 22.75 | 6.51 | 26.09 | 18.39 |
| 7-2b | 24 | 283.92 | 0.00 | 1.43 | 1.03 | 6.77 | 0.47 | 48 | 168.66 | 0.13 | 22.90 | 6.63 | 25.35 | 18.20 |
| 7-2c | 24 | 284.15 | 0.00 | 1.37 | 1.01 | 6.39 | 0.58 | 48 | 165.33 | 0.16 | 23.50 | 6.91 | 25.31 | 18.39 |
| 7-2d | 24 | 283.60 | 0.00 | 0.64 | 1.45 | 6.98 | 0.47 | 48 | 151.63 | 0.14 | 26.56 | 8.41 | 23.61 | 22.48 |
| 6-12b | 72 | 2.36 | 0.39 | 96.69 | 26.61 | 21.94 | 30.73 | 96 | 0.06 | 0.34 | 107.17 | 28.11 | 13.72 | 16.54 |
| 6-12b | 72 | 2.96 | 0.24 | 95.40 | 25.82 | 21.55 | 31.67 | 96 | 0.05 | 0.36 | 105.92 | 27.76 | 14.34 | 17.60 |
| 7-4a | 72 | 10.79 | 0.20 | 90.53 | 24.88 | 21.25 | 31.38 | 96 | 2.22 | 0.33 | 98.88 | 26.42 | 21.01 | 19.94 |
| 7-4b | 72 | 11.16 | 0.21 | 90.96 | 24.87 | 20.80 | 31.16 | 96 | 2.74 | 0.28 | 98.84 | 26.13 | 20.56 | 20.39 |
| 7-1a | 72 | 26.78 | 0.20 | 80.01 | 17.14 | 27.68 | 32.23 | 96 | 0.13 | 0.35 | 100.68 | 18.37 | 24.55 | 22.00 |
| 7-1b | 72 | 14.37 | 0.22 | 87.90 | 18.32 | 27.00 | 32.15 | 96 | 0.07 | 0.40 | 104.16 | 18.92 | 22.85 | 20.21 |
| 7-5a | 72 | 18.57 | 0.34 | 87.63 | 22.02 | 18.55 | 34.04 | 96 | 0.74 | 0.59 | 100.70 | 23.82 | 17.65 | 20.14 |
| 7-5b | 72 | 5.59 | 0.51 | 94.75 | 20.98 | 14.88 | 38.43 | 96 | 0.30 | 0.62 | 101.13 | 21.76 | 14.16 | 23.92 |
| 7-5c | 72 | 4.87 | 0.55 | 95.52 | 21.04 | 14.91 | 37.95 | 96 | 0.08 | 0.63 | 102.36 | 21.77 | 12.54 | 25.00 |
| 7-5d | 72 | 5.79 | 0.61 | 91.55 | 21.64 | 15.15 | 36.15 | 96 | 0.75 | 0.65 | 98.27 | 22.00 | 14.91 | 23.87 |
| 7-2a | 72 | 16.38 | 0.48 | 83.22 | 17.65 | 27.41 | 33.95 | 96 | 0.07 | 0.62 | 99.41 | 17.17 | 24.46 | 21.15 |
| 7-2b | 72 | 21.86 | 0.42 | 82.14 | 16.30 | 26.83 | 34.26 | 96 | 0.10 | 0.61 | 98.69 | 17.50 | 24.72 | 21.86 |
| 7-2c | 72 | 23.93 | 0.35 | 81.69 | 16.07 | 26.05 | 33.20 | 96 | 0.13 | 0.63 | 99.68 | 17.55 | 23.88 | 22.90 |
| 7-2d | 72 | 13.24 | 0.53 | 85.95 | 17.15 | 24.10 | 35.44 | 96 | 0.17 | 0.61 | 98.35 | 17.98 | 22.91 | 23.37 |

### Example 33: Shake Flask Fermentation Assay

Strains 6-12b, 7-5c, 7-6a, 7-6b, 7-6c, and 7-8 were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

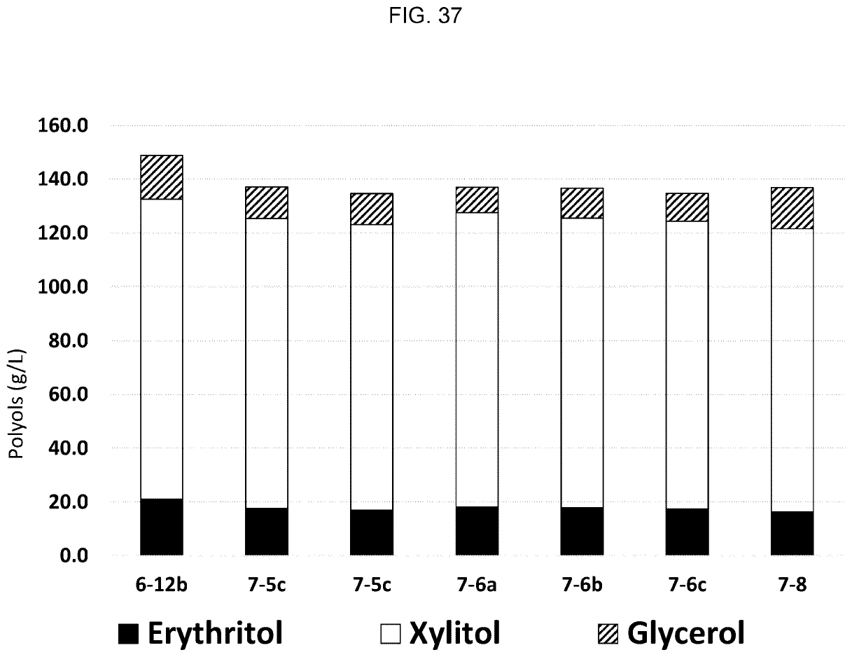

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Results are shown in Table 45 and results for the 96-hour time point are also shown in FIG. 37.

Overall, strains 7-5c, 7-6a, 7-6b, 7-6c, and 7-8 showed reduced erythritol production relative to strain 6-12b. However, knockout of the of the second allele of RCSR26640 (i.e., strains 7-6a, 7-6b, and 7-6c) did not further decrease erythritol production beyond the single knock out strains (7-5c and 7-8). Strain 7-8 does demonstrate that knocking out one allele of RCSR26640 by replacing it with a gene encoding an XPDH is effective at reducing erythritol production similar to the single knock out strain 7-5c.

TABLE 45

| | Time | | | | | | | Time | | | | | | |
|---|---|---|---|---|---|---|---|---|---|---|---|---|---|---|
| | Point | Fermentation Broth Analyte (g/L) | | | | | | Point | Fermentation Broth Analyte (g/L) | | | | | |
| Strain | (h) | Glucose | Ribitol | Xylitol | Erythritol | Glycerol | Ethanol | (h) | Glucose | Ribitol | Xylitol | Erythritol | Glycerol | Ethanol |
| 6-12b | 24 | 281.32 | 0.06 | 2.00 | 1.78 | 7.71 | 0.30 | 48 | 164.97 | 0.10 | 27.56 | 10.14 | 22.88 | 17.67 |
| 6-12b | 24 | 276.69 | 0.12 | 2.16 | 1.78 | 7.83 | 0.54 | 48 | 163.13 | 0.11 | 27.72 | 10.27 | 23.25 | 17.31 |
| 7-5c | 24 | 274.89 | 0.09 | 2.33 | 2.03 | 7.97 | 1.10 | 48 | 138.80 | 0.20 | 33.07 | 10.71 | 19.22 | 24.24 |
| 7-5c | 24 | 280.60 | 0.14 | 1.75 | 1.61 | 6.05 | 0.67 | 48 | 158.76 | 0.20 | 27.93 | 9.10 | 17.71 | 22.30 |
| 7-6a | 24 | 280.22 | 0.11 | 1.76 | 1.68 | 6.70 | 0.64 | 48 | 152.29 | 0.20 | 29.95 | 9.90 | 17.70 | 23.06 |
| 7-6b | 24 | 275.76 | 0.14 | 2.16 | 1.84 | 7.27 | 0.91 | 48 | 143.77 | 0.24 | 31.32 | 10.56 | 19.16 | 23.98 |
| 7-6c | 24 | 275.04 | 0.11 | 2.14 | 1.91 | 7.18 | 0.99 | 48 | 144.19 | 0.19 | 31.46 | 10.46 | 17.68 | 24.32 |
| 7-8 | 24 | 276.66 | 0.10 | 2.06 | 1.53 | 7.85 | 0.95 | 48 | 153.05 | 0.22 | 28.48 | 8.94 | 21.23 | 22.08 |
| 6-12b | 72 | 4.32 | 0.28 | 94.16 | 26.60 | 23.15 | 31.19 | 96 | 0.06 | 0.33 | 111.56 | 21.06 | 16.27 | 16.88 |
| 6-12b | 72 | 4.96 | 0.27 | 92.17 | 27.42 | 23.26 | 30.66 | 96 | 0.08 | 0.33 | 111.17 | 21.32 | 16.55 | 16.53 |
| 7-5c | 72 | 1.34 | 0.60 | 96.36 | 23.02 | 15.57 | 34.06 | 96 | 0.03 | 0.71 | 107.73 | 17.63 | 11.72 | 20.31 |
| 7-5c | 72 | 4.53 | 0.53 | 92.37 | 22.95 | 15.27 | 37.31 | 96 | 0.06 | 0.70 | 106.23 | 16.95 | 11.55 | 24.64 |
| 7-6a | 72 | 3.06 | 0.49 | 95.14 | 24.08 | 13.94 | 35.16 | 96 | 0.05 | 0.70 | 109.51 | 18.12 | 9.36 | 19.04 |
| 7-6b | 72 | 2.09 | 0.56 | 95.01 | 23.97 | 14.75 | 34.70 | 96 | 0.08 | 0.68 | 107.67 | 17.89 | 11.11 | 22.25 |
| 7-6c | 72 | 2.51 | 0.50 | 95.23 | 23.68 | 13.44 | 36.24 | 96 | 0.09 | 0.68 | 106.91 | 17.50 | 10.35 | 23.91 |
| 7-8 | 72 | 3.41 | 0.60 | 93.17 | 21.11 | 18.92 | 35.58 | 96 | 0.07 | 0.73 | 105.40 | 16.24 | 15.23 | 23.15 |

### Example 34: Shake Flask Fermentation Assay

Strains 6-12b, 6-12b, 7-2a, 7-2c, 7-3a-b, 7-9a-d, 7-10, 7-11a-d, 7-12a-f, and 7-13a-b were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

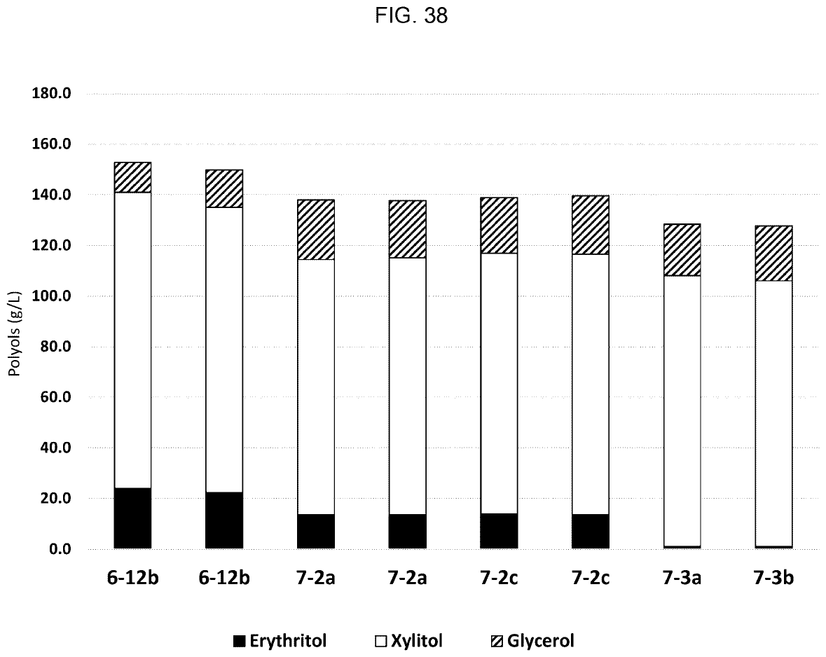

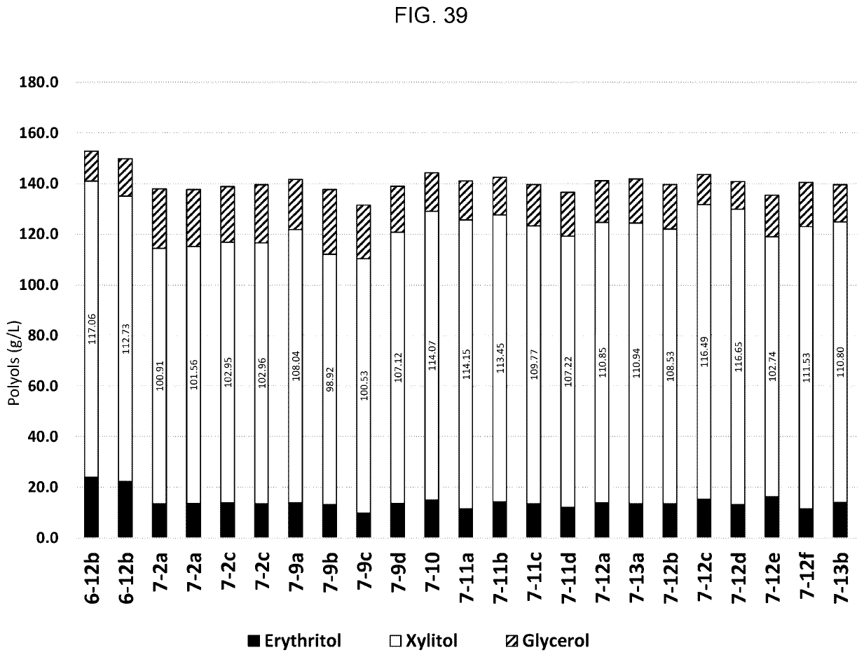

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Results are shown in Table 46. Results for the 96-hour time point comparing strains 7-2a, 7-2c, 7-3a, and 7-3b are shown in FIG. 38. Results for strains 6-12b, 7-2a, 7-2c, 7-9a-d, 7-10, 7-11a-d, 7-12a-f, and 7-13a-b at 96-hours are shown in FIG. 39.

Overall, strains 7-3a and 7-3b showed significant reduction in erythritol production, with the erythritol titer at 96 hours reduced to about 1.0 g/L. The two allele knock out of the RCSR18717 gene in these strains shows significant erythritol reduction relative to both the single knock out strains (7-2a and 7-2c) and to the grandparent strain (6-12b) which includes both wild-type alleles of RCSR18717.

TABLE 46

| | Time | | | | | | | Time | | | | | | |
|---|---|---|---|---|---|---|---|---|---|---|---|---|---|---|
| | Point | Fermentation Broth Analyte (g/L) | | | | | | Point | Fermentation Broth Analyte (g/L) | | | | | |
| Strain | (h) | Glucose | Ribitol | Xylitol | Erythritol | Glycerol | Ethanol | (h) | Glucose | Ribitol | Xylitol | Erythritol | Glycerol | Ethanol |
| 6-12b | 24 | 286.01 | 0.08 | 2.02 | 1.22 | 5.60 | 0.47 | 48 | 164.38 | 0.12 | 31.21 | 8.03 | 21.35 | 17.94 |
| 6-12b | 24 | 284.78 | 0.08 | 2.35 | 1.31 | 6.19 | 0.66 | 48 | 159.79 | 0.14 | 31.96 | 8.34 | 22.55 | 18.28 |
| 7-2a | 24 | 285.37 | 0.07 | 1.88 | 0.95 | 6.27 | 0.96 | 48 | 172.15 | 0.18 | 24.29 | 5.27 | 24.56 | 18.29 |
| 7-2a | 24 | 283.95 | 0.07 | 1.91 | 0.98 | 6.51 | 0.96 | 48 | 168.92 | 0.18 | 25.42 | 5.42 | 23.92 | 19.14 |
| 7-2c | 24 | 283.47 | 0.07 | 1.77 | 0.99 | 6.40 | 0.87 | 48 | 168.55 | 0.17 | 25.37 | 5.51 | 23.82 | 19.52 |
| 7-2c | 24 | 284.77 | 0.09 | 1.73 | 0.98 | 6.46 | 0.87 | 48 | 169.29 | 0.17 | 25.43 | 5.55 | 25.00 | 18.71 |
| 7-9a | 24 | 321.16 | 0.09 | 2.13 | 0.90 | 6.44 | 0.67 | 48 | 175.12 | 0.15 | 25.94 | 5.17 | 22.40 | 18.18 |
| 7-9b | 24 | 283.46 | 0.08 | 1.73 | 0.87 | 6.39 | 1.17 | 48 | 167.66 | 0.16 | 25.78 | 5.42 | 25.44 | 19.26 |
| 7-9c | 24 | 283.85 | 0.06 | 2.03 | 0.73 | 6.35 | 0.95 | 48 | 160.34 | 0.17 | 29.50 | 4.65 | 23.52 | 20.93 |
| 7-9d | 24 | 285.76 | 0.08 | 1.83 | 0.78 | 5.51 | 0.74 | 48 | 181.93 | 0.15 | 23.69 | 4.85 | 22.28 | 16.84 |
| 7-10 | 24 | 285.96 | 0.08 | 1.91 | 0.81 | 5.73 | 0.81 | 48 | 178.44 | 0.14 | 24.87 | 5.13 | 24.49 | 16.16 |
| 7-11a | 24 | 288.28 | 0.08 | 2.27 | 0.72 | 5.39 | 0.85 | 48 | 171.16 | 0.10 | 30.64 | 5.16 | 19.21 | 19.44 |
| 7-11b | 24 | 285.36 | 0.08 | 2.12 | 0.81 | 5.87 | 0.81 | 48 | 168.86 | 0.12 | 28.80 | 5.67 | 22.54 | 18.66 |
| 7-11c | 24 | 285.81 | 0.10 | 2.11 | 0.77 | 5.81 | 0.87 | 48 | 169.57 | 0.11 | 28.15 | 5.49 | 22.09 | 18.96 |
| 7-11d | 24 | 275.50 | 0.06 | 3.13 | 1.08 | 7.67 | 1.40 | 48 | 145.30 | 0.13 | 35.49 | 6.61 | 20.77 | 23.21 |
| 7-12a | 24 | 285.84 | 0.07 | 2.23 | 0.78 | 5.52 | 0.77 | 48 | 175.85 | 0.13 | 27.55 | 5.28 | 21.32 | 18.12 |
| 7-13a | 24 | 287.93 | 0.07 | 1.94 | 0.69 | 5.20 | 0.76 | 48 | 183.46 | 0.14 | 24.36 | 4.73 | 22.79 | 15.78 |
| 7-3a | 24 | 287.66 | 0.08 | 1.71 | 0.04 | 4.88 | 1.06 | 48 | 190.43 | 0.16 | 21.70 | 0.29 | 20.62 | 17.88 |
| 7-3b | 24 | 286.59 | 0.06 | 1.86 | 0.04 | 5.15 | 1.13 | 48 | 188.54 | 0.14 | 22.41 | 0.29 | 21.55 | 17.22 |
| 7-12b | 24 | 286.85 | 0.06 | 2.08 | 0.76 | 5.36 | 0.68 | 48 | 178.77 | 0.13 | 25.79 | 5.08 | 21.84 | 16.96 |
| 7-12c | 24 | 289.40 | 0.11 | 1.95 | 0.80 | 5.43 | 0.58 | 48 | 175.94 | 0.12 | 27.61 | 5.25 | 21.05 | 18.42 |
| 7-12d | 24 | 284.75 | 0.08 | 2.63 | 1.05 | 6.05 | 0.65 | 48 | 163.56 | 0.13 | 33.20 | 6.02 | 18.19 | 20.79 |
| 7-12e | 24 | 288.09 | 0.08 | 2.06 | 0.65 | 4.58 | 0.66 | 48 | 186.82 | 0.12 | 25.31 | 5.32 | 17.57 | 16.07 |
| 7-12f | 24 | 284.53 | 0.08 | 2.25 | 0.65 | 5.46 | 0.79 | 48 | 186.17 | 0.09 | 25.16 | 3.87 | 20.27 | 15.39 |
| 7-13b | 24 | 286.01 | 0.10 | 2.06 | 0.78 | 5.70 | 0.70 | 48 | 172.46 | 0.15 | 27.56 | 5.29 | 22.89 | 18.09 |
| 6-12b | 72 | 4.63 | 0.28 | 102.72 | 22.85 | 21.71 | 28.57 | 96 | 0.01 | 0.46 | 117.06 | 23.94 | 11.81 | 12.09 |
| 6-12b | 72 | 4.59 | 0.25 | 101.92 | 21.41 | 22.81 | 30.89 | 96 | 0.04 | 0.46 | 112.73 | 22.26 | 14.85 | 18.21 |
| 7-2a | 72 | 30.34 | 0.50 | 83.75 | 13.15 | 26.15 | 35.14 | 96 | 0.43 | 0.80 | 100.91 | 13.50 | 23.53 | 26.35 |
| 7-2a | 72 | 26.50 | 0.49 | 84.76 | 13.04 | 24.82 | 35.27 | 96 | 0.35 | 0.80 | 101.56 | 13.57 | 22.50 | 25.89 |
| 7-2c | 72 | 28.75 | 0.50 | 84.97 | 13.06 | 24.59 | 34.03 | 96 | 0.33 | 0.78 | 102.95 | 13.89 | 21.96 | 25.08 |
| 7-2c | 72 | 28.51 | 0.51 | 85.88 | 13.13 | 26.11 | 33.22 | 96 | 0.29 | 0.79 | 102.96 | 13.56 | 22.98 | 23.81 |
| 7-9a | 72 | 20.69 | 0.33 | 92.61 | 13.27 | 22.81 | 35.25 | 96 | 0.11 | 0.59 | 108.04 | 13.82 | 19.81 | 22.76 |
| 7-9b | 72 | 31.98 | 0.49 | 81.29 | 12.64 | 26.65 | 34.44 | 96 | 1.21 | 0.79 | 98.92 | 13.17 | 25.53 | 26.70 |
| 7-9c | 72 | 15.72 | 0.33 | 94.56 | 9.88 | 22.21 | 37.02 | 96 | 6.53 | 0.50 | 100.53 | 9.82 | 21.12 | 25.58 |
| 7-9d | 72 | 28.98 | 0.34 | 86.92 | 12.89 | 22.31 | 34.88 | 96 | 0.13 | 0.62 | 107.12 | 13.66 | 18.12 | 25.50 |
| 7-10 | 72 | 30.89 | 0.32 | 88.13 | 14.13 | 24.94 | 30.67 | 96 | 0.03 | 0.60 | 114.07 | 14.96 | 15.26 | 21.13 |
| 7-11a | 72 | 16.28 | 0.28 | 101.21 | 11.24 | 17.47 | 35.46 | 96 | 0.21 | 0.47 | 114.15 | 11.44 | 15.48 | 23.89 |
| 7-11b | 72 | 15.03 | 0.33 | 97.39 | 13.75 | 21.93 | 33.86 | 96 | 0.03 | 0.53 | 113.45 | 14.24 | 14.75 | 21.78 |
| 7-11c | 72 | 18.99 | 0.32 | 95.17 | 13.20 | 21.86 | 35.61 | 96 | 0.06 | 0.53 | 109.77 | 13.52 | 16.28 | 24.96 |
| 7-11d | 72 | 9.11 | 0.35 | 100.33 | 11.86 | 18.89 | 38.27 | 96 | 0.61 | 0.51 | 107.22 | 11.99 | 17.36 | 27.86 |
| 7-12a | 72 | 15.73 | 0.30 | 95.72 | 13.13 | 21.13 | 34.55 | 96 | 0.04 | 0.53 | 110.85 | 13.81 | 16.47 | 19.52 |
| 7-13a | 72 | 27.48 | 0.32 | 91.56 | 12.97 | 23.35 | 32.66 | 96 | 0.05 | 0.53 | 110.94 | 13.50 | 17.40 | 22.48 |
| 7-3a | 72 | 61.22 | 0.42 | 76.53 | 0.66 | 21.69 | 33.27 | 96 | 1.96 | 0.81 | 107.01 | 1.04 | 20.33 | 30.61 |
| 7-3b | 72 | 63.59 | 0.35 | 75.71 | 0.62 | 22.96 | 31.56 | 96 | 6.92 | 0.73 | 105.02 | 1.01 | 21.73 | 29.35 |
| 7-12b | 72 | 21.71 | 0.26 | 93.30 | 13.02 | 21.65 | 35.30 | 96 | 0.12 | 0.52 | 108.53 | 13.46 | 17.57 | 24.33 |
| 7-12c | 72 | 10.41 | 0.26 | 99.53 | 14.50 | 21.20 | 31.68 | 96 | 0.02 | 0.52 | 116.49 | 15.21 | 11.84 | 13.39 |
| 7-12d | 72 | 13.07 | 0.32 | 103.59 | 12.92 | 15.66 | 35.77 | 96 | 0.07 | 0.50 | 116.65 | 13.19 | 10.99 | 22.95 |
| 7-12e | 72 | 35.14 | 0.32 | 85.05 | 15.50 | 18.58 | 33.36 | 96 | 4.28 | 0.46 | 102.74 | 16.29 | 16.37 | 25.06 |
| 7-12f | 72 | 29.11 | 0.30 | 92.82 | 10.92 | 19.62 | 34.66 | 96 | 1.00 | 0.43 | 111.53 | 11.44 | 17.54 | 26.31 |
| 7-13b | 72 | 17.54 | 0.36 | 94.64 | 13.65 | 22.32 | 34.74 | 96 | 0.03 | 0.54 | 110.80 | 14.01 | 14.68 | 23.59 |

### Example 35: Shake Flask Fermentation Assay

Strains 6-12b and 7-14a-e were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30 °C for 48-72 hours. Cells from the incubated YPD plates were scraped into 40 mL rich medium (170 g/L glucose, 10 g/L yeast extract) in a 250 mL non-baffled flask. Cells were incubated at 30 °C and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

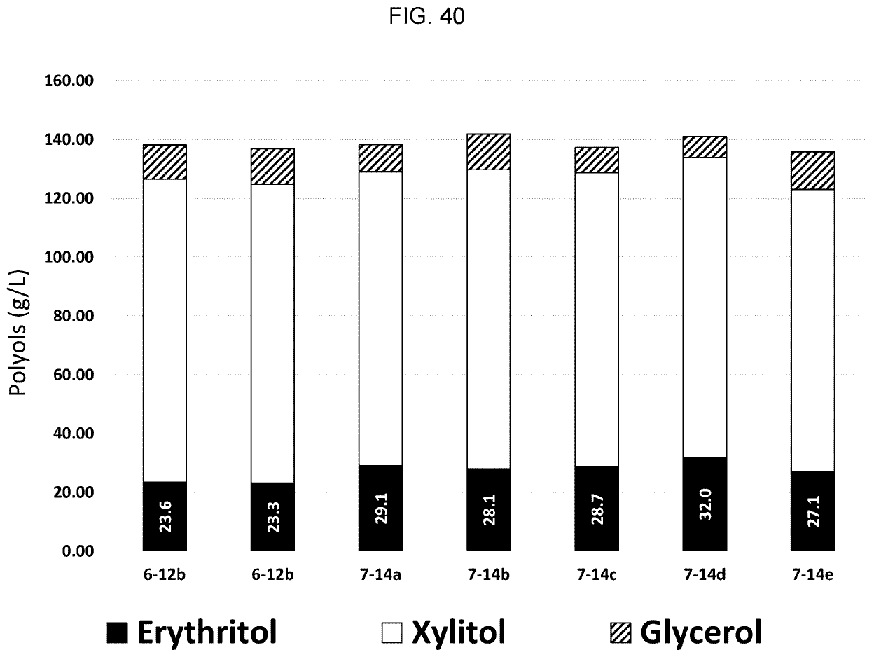

A 250 ml non-baffled flask containing 20 mL production medium (Table 5) was inoculated with 0.4 mL of the seed culture to form the production culture. The production culture was incubated at 35 °C and 250 rpm. Samples were taken from the production culture after 72 and 96 hours of incubation. Samples were analyzed for glucose, ribitol, xylitol, erythritol, glycerol, and ethanol by high performance liquid chromatography with refractive index detector. Results are shown in Table 47. Results for the 96-hour time point are shown in FIG. 40. Overall, strains 7-14a-e, which are engineered to overexpress the RCSR18717 gene, showed an increase in erythritol production relative to control strain 6-12b, which has wild-type levels of RCSR18717 expression. Strains 7-14a-e showed an approximately 25% increase in erythritol titer relative to strain 6-12b with no change in xylitol titer.

TABLE 47

| | Time | | | | | | | Time | | | | | | |
|---|---|---|---|---|---|---|---|---|---|---|---|---|---|---|
| | Point | Fermentation Broth Analyte (g/L) | | | | | | Point | Fermentation Broth Analyte (g/L) | | | | | |
| Strain | (h) | Glucose | Ribitol | Xylitol | Erythritol | Glycerol | Ethanol | (h) | Glucose | Ribitol | Xylitol | Erythritol | Glycerol | Ethanol |
| 6-12b | 72 | 2.02 | 0.25 | 91.59 | 22.56 | 18.46 | 30.47 | 96 | 0.07 | 0.34 | 103.06 | 23.56 | 11.57 | 16.27 |
| 6-12b | 72 | 2.98 | 0.34 | 91.51 | 22.05 | 18.54 | 31.40 | 96 | 0.02 | 0.47 | 101.55 | 23.27 | 12.04 | 19.83 |
| 7-14a | 72 | 0.70 | 0.31 | 93.31 | 27.79 | 14.52 | 30.92 | 96 | 0.03 | 0.44 | 99.97 | 29.12 | 9.31 | 19.48 |
| 7-14b | 72 | 4.26 | 0.32 | 91.81 | 27.32 | 15.46 | 30.62 | 96 | 0.05 | 0.36 | 101.72 | 28.08 | 12.11 | 20.42 |
| 7-14c | 72 | 0.68 | 0.32 | 93.48 | 27.71 | 13.57 | 32.05 | 96 | 0.04 | 0.37 | 100.07 | 28.68 | 8.55 | 19.51 |
| 7-14d | 72 | 0.32 | 0.34 | 93.09 | 30.28 | 15.02 | 28.44 | 96 | 0.04 | 0.42 | 102.00 | 31.97 | 7.05 | 16.15 |
| 7-14e | 72 | 0.67 | 0.30 | 90.28 | 25.86 | 18.34 | 30.23 | 96 | 0.04 | 0.40 | 96.03 | 27.09 | 12.74 | 18.77 |

1. A genetically engineered yeast cell capable of producing xylitol, the engineered yeast cell comprising:
a deletion or disruption of a native gene encoding an erythrose reductase enzyme at least 80% identical to at least one of SEQ ID NOs: 292 and 295.

2. The yeast cell of claim 1, wherein the yeast cell is an osmotolerant yeast cell.

3. The yeast cell of claim 1, wherein the yeast cell is a cell of the subphylum Ustilaginomycotina.

4. The yeast cell of claim 1, wherein the yeast cell is selected from the group consisting of Trichosporonoides megachiliensis, Trychosporonoides oedocephalis, Trychosporonoides nigrescens, Pseudozyma tsukubaensis, Trigonopsis variabilis, Moniliella, Ustilaginomycetes, Trichosporon, Yarrowia lipolytica, Saccharomyces cerevisiae, Penicillium, Torula, Pichia, Candida, Candida magnoliae, and Aureobasidium.

5. The yeast cell of claim 1, wherein the yeast cell is a Moniliella pollinis cell and the gene encoding the erythrose reductase enzyme is at least 60%, at least 65%, at least 70%, at least 75%, at least 80%, at least 85%, at least 90%, at least 95%, at least 98%, at least 99%, or 100% identical to at least one of SEQ ID NOs: 290 and 293.

6. The yeast cell of claim 5, wherein the cell has a deletion of at least one allele of the gene encoding the erythrose reductase enzyme.

7. The yeast of claim 5, wherein the cell has a deletion of both alleles of the gene encoding the erythrose reductase enzyme.

8. The yeast cell of claim 1, wherein the cell additionally comprises an exogenous polynucleotide sequence encoding a xylitol-phosphate dehydrogenase (XPDH) enzyme comprising a sequence at least 80% identical to at least one of SEQ ID NOs: 12-15, 28-31, and 33.

9. The yeast cell of claim 8, wherein the XPDH enzyme has a sequence at least 85% identical to at least one of SEQ ID NOs: 14, 15, 28, or 31.

10. The yeast cell of claim 8, wherein the XPDH enzyme has a sequence at least 90% identical to at least one of SEQ ID NOs: 14, 15, 28, or 31.

11. The yeast cell of claim 1, wherein the cell additionally comprises an exogenous polynucleotide sequence encoding a xylulokinase (XKS) enzyme comprising a sequence at least 80% identical to at least one of SEQ ID NOs: 188 and 189 and comprises an exogenous polynucleotide sequence encoding a xylitol dehydrogenase (XDH) enzyme comprising a sequence at least 80% identical to at least one of SEQ ID NOs: 190, 191, and 192.

12. (canceled)

13. The yeast cell of claim 1, wherein the cell additionally comprises a genetic modification resulting in overexpression of a native enzyme with ribulose-5-phosphate epimerase (RPE) activity.

14. The yeast cell of claim 13, wherein the cell is a Moniliella pollinis cell and the native RPE enzyme comprises a sequence at least 80% identical to at least one of SEQ ID NOs: 179 and 180.

15. The yeast cell of claim 13, wherein the genetic modification resulting in overexpression of a native RPE enzyme comprises addition of an exogenous polynucleotide encoding the native RPE enzyme such that the genetically engineered cell comprises at least one additional copy of a sequence encoding the RPE enzyme.

16. The yeast cell of claim 1, wherein the yeast additionally comprises a genetic modification resulting in overexpression of a native enzyme with xylitol-5-phosphate phosphatase (X5PP) activity; and/or an exogenous polynucleotide sequence encoding an enzyme with xylitol-5-phosphate phosphatase (X5PP) activity.

17. The yeast cell of claim 16, wherein the cell is a Moniliella pollinis cell and the genetic modification results in overexpression of a native X5PP enzyme with a sequence at least 80% identical to at least one of SEQ ID NOs: 198, 199, 200, or 221.

18. The yeast cell of claim 16, wherein the genetic modification comprises addition of an exogenous polynucleotide sequence encoding the native X5PP enzyme such that the genetically engineered cell comprises at least one additional copy of a sequence encoding the native X5PP enzyme.

19. The yeast cell of claim 16, wherein the yeast cell comprises an exogenous polynucleotide sequence encoding an enzyme with X5PP activity and a sequence at least 80% identical to at least one of SEQ ID NOs: 200, 201, 202, 203, 204, 205, 206, 207, 208, 209, 210, 211, 213, 214, 221, 222, 189, and 188.

20. (canceled)

21. (canceled)

22. (canceled)

23. (canceled)

24. (canceled)

25. (canceled)

26. (canceled)

27. (canceled)

28. A method for producing xylitol comprising
contacting a substrate comprising dextrose with the engineered yeast cell of claim 1, wherein fermentation of the substrate by the engineered cell produces xylitol.

29. The method of claim 28, wherein the fermentation temperature is at or between 25 °C to 45 °C and the volumetric oxygen uptake rate (OUR) is between 5-80 mmol O₂/(L·h).

30. The method of claim 28, wherein the xylitol is produced at a rate of at least 0.2 g L⁻¹ h⁻¹.

31. The method of claim 28, wherein the xylitol titer is at least 20 g/L when the fermentation is run at 35 °C for 96 hours.

32. The method of claim 28, wherein rate, titer, and/or yield of erythritol production is decreased relative to an equivalent fermentation run with an equivalent yeast cell in which the native gene encoding the erythrose reductase enzyme has not been deleted or disrupted.

33. The method of claim 28, wherein the concentration of dextrose is at least 100 g/L.

34. The method of claim 28, wherein the erythritol titer is less than 20 g/L when the fermentation is run at 35 °C for 96 hours; wherein the erythritol production rate is less than 0.5 g L⁻¹ h⁻¹; and/or wherein the erythritol yield is less than 35%.

35. (canceled)
